SEQUENCE LISTING (44 sequences)

SEQ ID NO: 1 - protein, 493 aa, Simmondsia chinensis
MEEMGSILEFLDNKAILVTGATGSLAKIFVEKVLRSQPNVKKLYLLLRATDDETAALRLQNEVFGKELFKVLKQNLGANFYSFVSEKVTVVPGDITGEDLCLKDVNLKEEMWREIDVVVNLAATINFIERYDVSLLINTYGAKYVLDFAKKCNKLKIFVHVSTAYVSGEKNGLILEKPYYMGESLNGRLGLDINVEKKLVEAKINELQAAGATEKSIKSTMKDMGIERARHWGWPNVYVFTKALGEMLLMQYKGDIPLTIIRPTIITSTFKEPFPGWVEGVRTIDNVPVYYGKGRLRCMLCGPSTIIDLIPADMVVNATIVAMVAHANQRYVEPVTYHVGSSAANPMKLSALPEMAHRYFTKNPWINPDRNPVHVGRAMVFSSFSTFHLYLTLNFLLPLKVLEIANTIFCQWFKGKYMDLKRKTRLLLRLVDIYKPYLFFQGIFDDMNTEKLRIAAKESIVEADMFYFDPRAINWEDYFLKTHFPGVVEHVLN

SEQ ID NO: 2 - protein, 352 aa, Simmondsia chinensis
MEVEKELKTFSEVWISAIAAACYCRFVPAVAPHGGALRLLLLLPVVLLFIFLPLRLSSFHLGGPTALYLVWLANFKLLLFAFHLGPLSNPSLSLLHFISTTLLPIKFRDDPSNDHEKNKRTLSFEWRKVVLFVAKLVFFAGILKIYEFRKDLPHFVISVLYCFHFYLGTEITLAASAVIARATLGLDLYPQFNEPYLATSLQDFWGRRWNLMVSDILGLTTYQPVRRVLSRWVRLRWEVAGAMLVAFTVSGLMHEVFFFYLTRARPSWEVTGFFVLHGVCTAVEMVVKKAVSGKVRLRREVSGALTVGFVMVTGGWLFLPQLVRHGVDLKTIDEYPVMFNYTQKKLMGLLGW

SEQ ID NO: 3 - DNA, 1479 bp, Simmondsia chinensis
atggaggagatgggcagcatcctggagttcctggacaacaaggccatcctggtgaccggcgccaccggcagcctggccaagatcttcgtggagaaggtgctgcgcagccagcccaacgtgaagaagctgtacctgctgctgcgcgccaccgacgacgagaccgccgccctgcgcctgcagaacgaggtgttcggcaaggagctgttcaaggtgctgaagcagaacctgggcgccaacttctacagcttcgtgagcgagaaggtgaccgtggtgcccggcgacatcaccggcgaggacctgtgcctgaaggacgtgaacctgaaggaggagatgtggcgcgagatcgacgtggtggtgaacctggccgccaccatcaacttcatcgagcgctacgacgtgagcctgctgatcaacacctacggcgccaagtacgtgctggacttcgccaagaagtgcaacaagctgaagatcttcgtgcacgtgagcaccgcctacgtgagcggcgagaagaacggcctgatcctggagaagccctactacatgggcgagagcctgaacggccgcctgggcctggacatcaacgtggagaagaagctggtggaggccaagatcaacgagctgcaggccgccggcgccaccgagaagagcatcaagagcaccatgaaggacatgggcatcgagcgcgcccgccactggggctggcccaacgtgtacgtgttcaccaaggccctgggcgagatgctgctgatgcagtacaagggcgacatccccctgaccatcatccgccccaccatcatcaccagcaccttcaaggagcccttccccggctgggtggagggcgtgcgcaccatcgacaacgtgcccgtgtactacggcaagggccgcctgcgctgcatgctgtgcggccccagcaccatcatcgacctgatccccgccgacatggtggtgaacgccaccatcgtggccatggtggcccacgccaaccagcgctacgtggagcccgtgacctaccacgtgggcagcagcgccgccaaccccatgaagctgagcgccctgcccgagatggcccaccgctacttcaccaagaacccctggatcaaccccgaccgcaaccccgtgcacgtgggccgcgccatggtgttcagcagcttcagcaccttccacctgtacctgaccctgaacttcctgctgcccctgaaggtgctggagatcgccaacaccatcttctgccagtggttcaagggcaagtacatggacctgaagcgcaagacccgcctgctgctgcgcctggtggacatctacaagccctacctgttcttccagggcatcttcgacgacatgaacaccgagaagctgcgcatcgccgccaaggagagcatcgtggaggccgacatgttctacttcgacccccgcgccatcaactgggaggactacttcctgaagacccacttccccggcgtggtggagcacgtgctgaac

SEQ ID NO: 4 - DNA, 1059 bp, Simmondsia chinensis
atggaggtggagaaggagctgaagaccttcagcgaggtgtggatcagcgccatcgccgccgcctgctactgccgcttcgtgcccgccgtggccccccacggcggcgccctgcgcctgctgctgctgctgcccgtggtgctgctgttcatcttcctgcccctgcgcctgagcagcttccacctgggcggccccaccgccctgtacctggtgtggctggccaacttcaagctgctgctgttcgccttccacctgggccccctgagcaaccccagcctgagcctgctgcacttcatcagcaccaccctgctgcccatcaagttccgcgacgaccccagcaacgaccacgagaagaacaagcgcaccctgagcttcgagtggcgcaaggtggtgctgttcgtggccaagctggtgttcttcgccggcatcctgaagatctacgagttccgcaaggacctgccccacttcgtgatcagcgtgctgtactgcttccacttctacctgggcaccgagatcaccctggccgccagcgccgtgatcgcccgcgccaccctgggcctggacctgtacccccagttcaacgagccctacctggccaccagcctgcaggacttctggggccgccgctggaacctgatggtgagcgacatcctgggcctgaccacctaccagcccgtgcgccgcgtgctgagccgctgggtgcgcctgcgctgggaggtggccggcgccatgctggtggccttcaccgtgagcggcctgatgcacgaggtgttcttcttctacctgacccgcgcccgccccagctgggaggtgaccggcttcttcgtgctgcacggcgtgtgcaccgccgtggagatggtggtgaagaaggccgtgagcggcaaggtgcgcctgcgccgcgaggtgagcggcgccctgaccgtgggcttcgtgatggtgaccggcggctggctgttcctgccccagctggtgcgccacggcgtggacctgaagaccatcgacgagtaccccgtgatgttcaactacacccagaagaagctgatgggcctgctgggctggtaa

SEQ ID NO: 5 - protein, 514 aa, Euglena gracilis
MNDFYAGKGVFLTGVTGFVGKMVVEKILRSLPTVGRLYVLVRPKAGTDPHQRLHSEVWSSAGFDVVREKVGGPAAFDALIREKVVPVPGDMVKDRFGLDDAAYRSLAANVNVIIHMAATIDFTERLDVAVSLNVLGTVRVLTLARRARELGALHSVVHVSTCYVNSNQPPGARLREQLYPLPFDPREMCTRILDMSPREIDLFGPQLLKQYGFPNTYTFTKCMAEQLGAQIAHDLPFAIFRPAIIGAALSEPFPGWCDSASACGAVFLAVGLGVLQELQGNASSVCDLIPVDHVVNMLLVTAAYTASAPPADPSPSSLALSPPQLPLATLPPGTVADVPIYHCGTSAGPNAVNWGRIKVSLVEYWNAHPIAKTKAAIALLPVWRFELSFLLKRRLPATALSLVASLPGASAAVRRQAEQTERLVGKMRKLVDTFQSFVFWAWYFQTESSARLLASLCPEDRETFNWDPRRIGWRAWVENYCYGLVRYVLKQPIGDRPPVAAEELASNRFLRAML

SEQ ID NO: 6 - protein, 368 aa, Euglena gracilis
MDFLGFPDSESERHAHFYVLASSFAAAIYMFTIPRRVKAGRKRFLLCSPVLLLNIMQPYIFFWTVGRHYCNFIPLYAAFCTWWTAFKVMAFGIGRGPLCQFSAFHKFAVVMLLPILPHGDTNHGVKDERSGSSWSSPTYLEMFAKFCGLGLCTYGISQLSHDGFPVLYNVFLSLIMYLHICVQYTGSNLATSKVLQVPLSDGMNQPYFSTSLSNFWGRRWNLVASSSLRHVVYDPIREGRLVPKGHPEEKPGGGKEVSRKVLGSLMAFLVSGIMHEYILWLATGFWSGQMLLFFVVHGVAVAAERVAKVAWARHGLPAIPCAVSIPMTIGFLFGTAELLFYPPIFSANWAEHGVADLRRQFRSLGLSV

SEQ ID NO: 7 - DNA, 1545 bp, Euglena gracilis
atgaacgacttctacgccggcaagggcgtgttcctgaccggcgtgaccggcttcgtgggcaagatggtggtggagaagatcctgcgcagcctgcccaccgtgggccgcctgtacgtgctggtgcgccccaaggccggcaccgacccccaccagcgcctgcacagcgaggtgtggagcagcgccggcttcgacgtggtgcgcgagaaggtgggcggccccgccgccttcgacgccctgatccgcgagaaggtggtgcccgtgcccggcgacatggtgaaggaccgcttcggcctggacgacgccgcctaccgcagcctggccgccaacgtgaacgtgatcatccacatggccgccaccatcgacttcaccgagcgcctggacgtggccgtgagcctgaacgtgctgggcaccgtgcgcgtgctgaccctggcccgccgcgcccgcgagctgggcgccctgcacagcgtggtgcacgtgagcacctgctacgtgaacagcaaccagccccccggcgcccgcctgcgcgagcagctgtaccccctgcccttcgacccccgcgagatgtgcacccgcatcctggacatgagcccccgcgagatcgacctgttcggcccccagctgctgaagcagtacggcttccccaacacctacaccttcaccaagtgcatggccgagcagctgggcgcccagatcgcccacgacctgcccttcgccatcttccgccccgccatcatcggcgccgccctgagcgagcccttccccggctggtgcgacagcgccagcgcctgcggcgccgtgttcctggccgtgggcctgggcgtgctgcaggagctgcagggcaacgccagcagcgtgtgcgacctgatccccgtggaccacgtggtgaacatgctgctggtgaccgccgcctacaccgccagcgccccccccgccgaccccagccccagcagcctggccctgagccccccccagctgcccctggccaccctgccccccggcaccgtggccgacgtgcccatctaccactgcggcaccagcgccggccccaacgccgtgaactggggccgcatcaaggtgagcctggtggagtactggaacgcccaccccatcgccaagaccaaggccgccatcgccctgctgcccgtgtggcgcttcgagctgagcttcctgctgaagcgccgcctgcccgccaccgccctgagcctggtggccagcctgcccggcgccagcgccgccgtgcgccgccaggccgagcagaccgagcgcctggtgggcaagatgcgcaagctggtggacaccttccagagcttcgtgttctgggcctggtacttccagaccgagagcagcgcccgcctgctggccagcctgtgccccgaggaccgcgagaccttcaactgggacccccgccgcatcggctggcgcgcctgggtggagaactactgctacggcctggtgcgctacgtgctgaagcagcccatcggcgaccgcccccccgtggccgccgaggagctggccagcaaccgcttcctgcgcgccatgctgtaa

SEQ ID NO: 8 - DNA, 1107 bp, Euglena gracilis
atggacttcctgggcttccccgacagcgagagcgagcgccacgcccacttctacgtgctggccagcagcttcgccgccgccatctacatgttcaccatcccccgccgcgtgaaggccggccgcaagcgcttcctgctgtgcagccccgtgctgctgctgaacatcatgcagccctacatcttcttctggaccgtgggccgccactactgcaacttcatccccctgtacgccgccttctgcacctggtggaccgccttcaaggtgatggccttcggcatcggccgcggccccctgtgccagttcagcgccttccacaagttcgccgtggtgatgctgctgcccatcctgccccacggcgacaccaaccacggcgtgaaggacgagcgcagcggcagcagctggagcagccccacctacctggagatgttcgccaagttctgcggcctgggcctgtgcacctacggcatcagccagctgagccacgacggcttccccgtgctgtacaacgtgttcctgagcctgatcatgtacctgcacatctgcgtgcagtacaccggcagcaacctggccaccagcaaggtgctgcaggtgcccctgagcgacggcatgaaccagccctacttcagcaccagcctgagcaacttctggggccgccgctggaacctggtggccagcagcagcctgcgccacgtggtgtacgaccccatccgcgagggccgcctggtgcccaagggccaccccgaggagaagcccggcggcggcaaggaggtgagccgcaaggtgctgggcagcctgatggccttcctggtgagcggcatcatgcacgagtacatcctgtggctggccaccggcttctggagcggccagatgctgctgttcttcgtggtgcacggcgtggccgtggccgccgagcgcgtggccaaggtggcctgggcccgccacggcctgcccgccatcccctgcgccgtgagcatccccatgaccatcggcttcctgttcggcaccgccgagctgctgttctacccccccatcttcagcgccaactgggccgagcacggcgtggccgacctgcgccgccagttccgcagcctgggcctgagcgtgtaa

SEQ ID NO: 9 - protein, 526 aa, Euglena gracilis
MVVAETTPVANSISVGDLFWWRIDEPTNPMVISVILGMDGTISLAELRDALRPHVEDNIRLQGTPQPNGIYSWRPYFIASVLLSLVLGWALRSLCCFSYIVAFGLLVGIALETRTGRQWRWVKVKDFALEDHIKLHVLPEETLECLHGFIDELASTQLPRDRAQWMVYLIHNAPGGSRILFRFHHIVGDGAGLGIWFYNLCTNAEQKKQDMEARHELLAKSKARRAENRTKPSPLAKLDGFVSKVLLILGGTTKLLFLPRDSNSPVKGANVGKKKTAVTGKDLLFPLEEVKHVGKALHPNITVNDTMCALVGGAFRRYYQSLHLHPEQMLMRATVPINIRPSTTAPIKMENDFTIVFKSLPIHLPTPEERIAHFHVRMGFLKRGIEPLLSMFLQHLLTWLPEPLMRLIVLRFTICSSAVLTNVLSSTVPFSLCGQPLTTAAFWVPTSGDIGIGISIMTYCDTVAINFIADENLIADWAPVVQFMREEWEEMKGILGKEQHLPVMEPQKTVELVNLWRTWGFPWNTR

SEQ ID NO: 10 - DNA, 1578 bp, Euglena gracilis
atggtggtggccgagaccacccccgtggccaacagcatcagcgtgggcgacctgttctggtggcgcatcgacgagcccaccaaccccatggtgatcagcgtgatcctgggcatggacggcaccatcagcctggccgagctgcgcgacgccctgcgcccccacgtggaggacaacatccgcctgcagggcaccccccagcccaacggcatctacagctggcgcccctacttcatcgccagcgtgctgctgagcctggtgctgggctgggccctgcgcagcctgtgctgcttcagctacatcgtggccttcggcctgctggtgggcatcgccctggagacccgcaccggccgccagtggcgctgggtgaaggtgaaggacttcgccctggaggaccacatcaagctgcacgtgctgcccgaggagaccctggagtgcctgcacggcttcatcgacgagctggccagcacccagctgccccgcgaccgcgcccagtggatggtgtacctgatccacaacgcccccggcggcagccgcatcctgttccgcttccaccacatcgtgggcgacggcgccggcctgggcatctggttctacaacctgtgcaccaacgccgagcagaagaagcaggacatggaggcccgccacgagctgctggccaagagcaaggcccgccgcgccgagaaccgcaccaagcccagccccctggccaagctggacggcttcgtgagcaaggtgctgctgatcctgggcggcaccaccaagctgctgttcctgccccgcgacagcaacagccccgtgaagggcgccaacgtgggcaagaagaagaccgccgtgaccggcaaggacctgctgttccccctggaggaggtgaagcacgtgggcaaggccctgcaccccaacatcaccgtgaacgacaccatgtgcgccctggtgggcggcgccttccgccgctactaccagagcctgcacctgcaccccgagcagatgctgatgcgcgccaccgtgcccatcaacatccgccccagcaccaccgcccccatcaagatggagaacgacttcaccatcgtgttcaagagcctgcccatccacctgcccacccccgaggagcgcatcgcccacttccacgtgcgcatgggcttcctgaagcgcggcatcgagcccctgctgagcatgttcctgcagcacctgctgacctggctgcccgagcccctgatgcgcctgatcgtgctgcgcttcaccatctgcagcagcgccgtgctgaccaacgtgctgagcagcaccgtgcccttcagcctgtgcggccagcccctgaccaccgccgccttctgggtgcccaccagcggcgacatcggcatcggcatcagcatcatgacctactgcgacaccgtggccatcaacttcatcgccgacgagaacctgatcgccgactgggcccccgtggtgcagttcatgcgcgaggagtgggaggagatgaagggcatcctgggcaaggagcagcacctgcccgtgatggagccccagaagaccgtggagctggtgaacctgtggcgcacctggggcttcccctggaacacccgc

SEQ ID NO: 11 - protein, 439 aa, Euglena gracilis
MVDSQPARPEGGARAVNRKLTKLGWSTLVTETSTNLSVPITIMVLETPITLPELYDILQERLLRQHSRYRSLVQGTGELVELPIEDVVLEQHVRVHQLGDPDSQRELNTVLGNLSCLPLVMTRPLWEVVLIPKFKSGSVLVFRNHHCLSDGGGGAIIVDSISDSPEQWEPKRKPALGEHILQLLALTLTLLASVPFVLYSVILVVLFPDRPSPLKPKQLEGGRRKVAISGPISVPALKKVCRANNCKINDLALTLYAQALRDQAKAIDPTFDKPVWSGIPVDVRLRGEVYTGNKFGFGVCRLPLHIAAFPEALAYVQKRMTFMKEHNLAMVMYYFSVVSSALMPTALLRAMLAFNTRRISLVVSNVAAGNKQLVLKGHAIQYMYALVPPPPNVGIGCSVIGQQDQLVFGMVVDSAAAIDPQAAIDHVLNALHVLSGGEI

SEQ ID NO: 12 - DNA, 1320 bp, Euglena gracilis
atggtggacagccagcccgcccgccccgagggcggcgcccgcgccgtgaaccgcaagctgaccaagctgggctggagcaccctggtgaccgagaccagcaccaacctgagcgtgcccatcaccatcatggtgctggagacccccatcaccctgcccgagctgtacgacatcctgcaggagcgcctgctgcgccagcacagccgctaccgcagcctggtgcagggcaccggcgagctggtggagctgcccatcgaggacgtggtgctggagcagcacgtgcgcgtgcaccagctgggcgaccccgacagccagcgcgagctgaacaccgtgctgggcaacctgagctgcctgcccctggtgatgacccgccccctgtgggaggtggtgctgatccccaagttcaagagcggcagcgtgctggtgttccgcaaccaccactgcctgagcgacggcggcggcggcgccatcatcgtggacagcatcagcgacagccccgagcagtgggagcccaagcgcaagcccgccctgggcgagcacatcctgcagctgctggccctgaccctgaccctgctggccagcgtgcccttcgtgctgtacagcgtgatcctggtggtgctgttccccgaccgccccagccccctgaagcccaagcagctggagggcggccgccgcaaggtggccatcagcggccccatcagcgtgcccgccctgaagaaggtgtgccgcgccaacaactgcaagatcaacgacctggccctgaccctgtacgcccaggccctgcgcgaccaggccaaggccatcgaccccaccttcgacaagcccgtgtggagcggcatccccgtggacgtgcgcctgcgcggcgaggtgtacaccggcaacaagttcggcttcggcgtgtgccgcctgcccctgcacatcgccgccttccccgaggccctggcctacgtgcagaagcgcatgaccttcatgaaggagcacaacctggccatggtgatgtactacttcagcgtggtgagcagcgccctgatgcccaccgccctgctgcgcgccatgctggccttcaacacccgccgcatcagcctggtggtgagcaacgtggccgccggcaacaagcagctggtgctgaagggccacgccatccagtacatgtacgccctggtgccccccccccccaacgtgggcatcggctgcagcgtgatcggccagcaggaccagctggtgttcggcatggtggtggacagcgccgccgccatcgacccccaggccgccatcgaccacgtgctgaacgccctgcacgtgctgagcggcggcgagatctaa

SEQ ID NO: 13 - protein, 517 aa, Euglena gracilis
MAVPGIKVSTKLTATDLFWWRVDEPQNPMVINILVEFEGVLTPAAVRDALEAAVAENIRLHGVPTSRFADTAGTWGLLAGCLTVLATGSQWYWKPIPHFSLEEHIRLHVLEERSEDCLRRFVDEEISHQLPKDRAQWRGIVIHNTPGSGSRALFRFHHVIADGAGLGQWFYGLCQVHGPPTGDSPHEVPEKQAWVGRHPSTLSAHPPPKRTAVQRLRKVAARVRDVVDFLLLEVLLVVYSALKLLFLSRDSNSPFKGPNTGRKKTGTTLHSLDLPVEAVKALGKGYDRDITVNDVLCTLLAGAFRRFFQRHLLHPEQMSMRVAVPINMRSSIRPPITMDNRFSLVFKSLPIHLPTVQERLASFHVRMGLMKMSIEPRLGLLLMYFLAWMPERVLARVIEHFTLCTSAVLTNVMSSRIKLSFAGQPMDNMCFWVPTSGDIGLGISVCTYCDRINLGLVVDENLLADVKPLLADVVAEWDDMQRQLSAQGAAHPSSVIPAHTQEMIEANQQYGKPGHSR

SEQ ID NO: 14 - DNA, 1551 bp, Euglena gracilis
atggccgtgcccggcatcaaggtgagcaccaagctgaccgccaccgacctgttctggtggcgcgtggacgagccccagaaccccatggtgatcaacatcctggtggagttcgagggcgtgctgacccccgccgccgtgcgcgacgccctggaggccgccgtggccgagaacatccgcctgcacggcgtgcccaccagccgcttcgccgacaccgccggcacctggggcctgctggccggctgcctgaccgtgctggccaccggcagccagtggtactggaagcccatcccccacttcagcctggaggagcacatccgcctgcacgtgctggaggagcgcagcgaggactgcctgcgccgcttcgtggacgaggagatcagccaccagctgcccaaggaccgcgcccagtggcgcggcatcgtgatccacaacacccccggcagcggcagccgcgccctgttccgcttccaccacgtgatcgccgacggcgccggcctgggccagtggttctacggcctgtgccaggtgcacggcccccccaccggcgacagcccccacgaggtgcccgagaagcaggcctgggtgggccgccaccccagcaccctgagcgcccaccccccccccaagcgcaccgccgtgcagcgcctgcgcaaggtggccgcccgcgtgcgcgacgtggtggacttcctgctgctggaggtgctgctggtggtgtacagcgccctgaagctgctgttcctgagccgcgacagcaacagccccttcaagggccccaacaccggccgcaagaagaccggcaccaccctgcacagcctggacctgcccgtggaggccgtgaaggccctgggcaagggctacgaccgcgacatcaccgtgaacgacgtgctgtgcaccctgctggccggcgccttccgccgcttcttccagcgccacctgctgcaccccgagcagatgagcatgcgcgtggccgtgcccatcaacatgcgcagcagcatccgcccccccatcaccatggacaaccgcttcagcctggtgttcaagagcctgcccatccacctgcccaccgtgcaggagcgcctggccagcttccacgtgcgcatgggcctgatgaagatgagcatcgagccccgcctgggcctgctgctgatgtacttcctggcctggatgcccgagcgcgtgctggcccgcgtgatcgagcacttcaccctgtgcaccagcgccgtgctgaccaacgtgatgagcagccgcatcaagctgagcttcgccggccagcccatggacaacatgtgcttctgggtgcccaccagcggcgacatcggcctgggcatcagcgtgtgcacctactgcgaccgcatcaacctgggcctggtggtggacgagaacctgctggccgacgtgaagcccctgctggccgacgtggtggccgagtgggacgacatgcagcgccagctgagcgcccagggcgccgcccaccccagcagcgtgatccccgcccacacccaggagatgatcgaggccaaccagcagtacggcaagcccggccacagccgc

SEQ ID NO: 15 - protein, 491 aa, Arabidopsis thaliana
MESNCVQFLGNKTILITGAPGFLAKVLVEKILRLQPNVKKIYLLLRAPDEKSAMQRLRSEVMEIDLFKVLRNNLGEDNLNALMREKIVPVPGDISIDNLGLKDTDLIQRMWSEIDIIINIAATTNFDERYDIGLGINTFGALNVLNFAKKCVKGQLLLHVSTAYISGEQPGLLLEKPFKMGETLSGDRELDINIEHDLMKQKLKELQDCSDEEISQTMKDFGMARAKLHGWPNTYVFTKAMGEMLMGKYRENLPLVIIRPTMITSTIAEPFPGWIEGLKTLDSVIVAYGKGRLKCFLADSNSVFDLIPADMVVNAMVAAATAHSGDTGIQAIYHVGSSCKNPVTFGQLHDFTARYFAKRPLIGRNGSPIIVVKGTILSTMAQFSLYMTLRYKLPLQILRLINIVYPWSHGDNYSDLSRKIKLAMRLVELYQPYLLFKGIFDDLNTERLRMKRKENIKELDGSFEFDPKSIDWDNYITNTHIPGLITHVLKQ

SEQ ID NO: 16 - protein, 480 aa, Arabidopsis thaliana
MKAEKVMEREIETTPIEPLSPMSHMLSSPNFFIVITFGFKTRCNRSAFVDGINNTLINAPRFSSKMEINYKKKGEPVWIPVKLRVDDHIIVPDLEYSNIQNPDQFVEDYTSNIANIPMDMSKPLWEFHLLNMKTSKAESLAIVKIHHSIGDGMSLMSLLLACSRKISDPDALVSNTTATKKPADSMAWWLFVGFWFMIRVTFTTIVEFSKLMLTVCFLEDTKNPLMGNPSDGFQSWKVVHRIISFEDVKLIKDTMNMKVNDVLLGMTQAGLSRYLSSKYDGSTAEKKKILEKLRVRGAVAINLRPATKIEDLADMMAKGSKCRWGNFIGTVIFPLWVKSEKDPLEYIRRAKATMDRKKISLEAFFFYGIIKFTLKFFGGKAVEAFGKRIFGHTSLAFSNVKGPDEEISFFHHPISYIAGSALVGAQALNIHFISYVDKIVINLAVDTTTIQDPNRLCDDMVEALEIIKSATQGEIFHKTE

SEQ ID NO: 17 - protein, 314 aa, Arabidopsis thaliana
MGGSREFRAEEHSNQFHSIIAMAIWLGAIHFNVALVLCSLIFLPPSLSLMVLGLLSLFIFIPIDHRSKYGRKLARYICKHACNYFPVSLYVEDYEAFQPNRAYVFGYEPHSVLPIGVVALCDLTGFMPIPNIKVLASSAIFYTPFLRHIWTWLGLTAASRKNFTSLLDSGYSCVLVPGGVQETFHMQHDAENVFLSRRRGFVRIAMEQGSPLVPVFCFGQARVYKWWKPDCDLYLKLSRAIRFTPICFWGVFGSPLPCRQPMHVVVGKPIEVTKTLKPTDEEIAKFHGQYVEALRDLFERHKSRVGYDLELKIL

SEQ ID NO: 18 - protein, 529 aa, Arabidopsis thaliana [sequence omitted]

SEQ ID NO: 19 - protein, 318 aa, Arabidopsis thaliana
MEICTYFKSQPTWLLILFVLGSISIFKFIFTLLRSFYIYFLRPSKNLRRYGSWAIITGPTDGIGKAFAFQLAQKGLNLILVARNPDKLKDVSDSIRSKYSQTQILTVVMDFSGDIDEGVKRIKESIEGLDVGILINNAGMSYPYAKYFHEVDEELINNLIKINVEGTTKVTQAVLPNMLKRKKGAIINMGSGAAALIPSYPFYSVYAGAKTYVDQFTKCLHVEYKKSGIDVQCQVPLYVATKMTKIRRASFLVASPEGYAKAALRFVGYEAQCTPYWPHALMGAVVSALPESVFESFNIKRCLQIRKKGLQKDSMKKE

SEQ ID NO: 20 - protein, 221 aa, Arabidopsis thaliana
MAGFLSVVRRVYLTLYNWIVFAGWAQVLYLAITTLKETGYENVYDAIEKPLQLAQTAAVLEILHGLVGLVRSPVSATLPQIGSRLFLTWGILYSFPEVRSHFLVTSLVISWSITEIIRYSFFGFKEALGFAPSWHLWLRYSSFLLLYPTGITSEVGLIYLALPHIKTSEMYSVRMPNILNFSFDFFYATILVLAIYVPGSPHMYRYMLGQRKRALSKSKRE

SEQ ID NO: 21 - protein, 310 aa, Arabidopsis thaliana
MKVTVVSRSGREVLKAPLDLPDSATVADLQEAFHKRAKKFYPSRQRLTLPVTPGSKDKPVVLNSKKSLKEYCDGNNNSLTVVFKDLGAQVSYRTLFFFEYLGPLLIYPVFYYFPVYKFLGYGEDCVIHPVQTYAMYYWCFHYFKRILETFFVHRFSHATSPIGNVFRNCAYYWSFGAYIAYYVNHPLYTPVSDLQMKIGFGFGLVCQVANFYCHILLKNLRDPSGAGGYQIPRGFLFNIVTCANYTTEIYQWLGFNIATQTIAGYVFLAVAALIMTNWALGKHSRLRKIFDGKDGKPKYPRRWVILPPFL

SEQ ID NO: 22 - DNA, 864 bp, Chlamydomonas reinhardtii
tcgagggtgccccgccagcccccgctcctctgctgcctctgatgcctcatgccaaaagtcctgacgcggcgccctcacatccccgtccgggtaatctatgagtttcccttatcgagcatgtacgcgatagtggacggggctcagggtggggggtgggtgggtgggaggggcgttccttcagacaccctggaggggtggctagaaaagcggccgcgcgccagaaatgtctcgctgccctgtgcaataagcaccggctatattgctcagcgctgttcggcgcaacggggggtcagcccttgggaagcgttggactatatggtagggtgcgagtgaccccgcgcgacttggagctcgatggccccgggttgtttggggcgtccgcctctcgcgctattctgagctggagaccgaggcgcatgaaaatgcattcgcttccataggacgctgcattgtggcttgaaggttcaagggaagggttcaaacgaccccgccgtacgaacttttgtcggggggcgctcccggccccgggctcttgtgcgcgcattagggcttcgggtcgcaagcaagacgatacaggaaccgaccaatcgatagtcttgtgcgaccgtgcacgtgtgcagcaatagttaggtcgataaccacgttgaacttgcgtctctcttcgtggcgcctcctgcttggtgctccacttcacttgtcgctatatagcacagcgttgaaagcaaaggccacactaatacagccgggctcgagagtccgtctgcgtttgcattgttggccaagggctgctttgtagccaaagccatacacgaagcttcacttgattagctttacgaccctcagccgaatcctgccagtgaattc

SEQ ID NO: 23 - DNA, 943 bp, Chlamydomonas reinhardtii
ctcgagctcgagcagaggttgggaatcgctttgaaaatccagcaatcgggtctcagctgtctcaggccgcacgcgccttggacaaggcacttcagtaacgtactccaagccctctatctgcatgcccacaaagcgcaggaatgccgaccatcgtgccagactgtgccgcgcccgaaccgaaatccgtcactccccttggttcccatggtggcatggtcccccctgttcgcccaaagcctggttcagcgcccagtggcaaacggctttggctcagctccttggtattgctggtttctagcaatctcgtccgttcctctgttgccaatgtagcaggtgcaaacagtcgaatacggttttactcaggggcaatctcaactaacagaggccctgggcctgttgcctggaacctatgaagacgataatgccacggcgactttcgagcctgagggaagtttgcaccggtaccgcattgtgcaaggttacggtacatgatagggggagtgcgacgcggtaaggcttggcgcagcttggcgcgtctgccttgcatgcatgtccgaaacacgccacgtcgcgccacgaaaagcggtaaaaggacctgccatggtcctccagggtgttaccacttccatttcgctcagctgggatggtgctcgtaggtgcaccagcgttgattatttcaggcaggaagcggctgcgaagcccgcctttcactgaagactgggatgagcgcacctgtacctgccagtatggtaccggcgcgctaccgatgcgtgtagtagagcttgctgccatacagtaactctggtacccccagccaccgggcgtagcgagcagactcaataagtatgatgggttcttattgcagccgctgttacagtttacagcgcaagggaacacgcccctcattcacagaactaactcaacctactccatcgacgaattc

SEQ ID NO: 24 - protein, 345 aa, Synechococcus elongatus
MEKTIGLEIIEVVEQAAIASARLMGKGEKNEADRVAVEAMRVRMNQVEMLGRIVIGEGERDEAPMLYIGEEVGIYRDADKRAGVPAGKLVEIDIAVDPCEGTNLCAYGQPGSMAVLAISEKGGLFAAPDFYMKKLAAPPAAKGKVDINKSATENLKILSECLDRAIDELVVVVMDRPRHKELIQEIRQAGARVRLISDGDVSAAISCGFAGTNTHALMGIGAAPEGVISAAAMRCLGGHFQGQLIYDPEVVKTGLIGESRESNIARLQEMGITDPDRVYDANELASGQEVLFAACGITPGLLMEGVRFFKGGARTQSLVISSQSRTARFVDTVHMFDDVKTVSLR

SEQ ID NO: 25 - DNA, 1035 bp, Synechococcus elongatus
atggagaagaccatcggcctggagatcatcgaggtggtggagcaggccgccatcgccagcgcccgcctgatgggcaagggcgagaagaacgaggccgaccgcgtggccgtggaggccatgcgcgtgcgcatgaaccaggtggagatgctgggccgcatcgtgatcggcgagggcgagcgcgacgaggcccccatgctgtacatcggcgaggaggtgggcatctaccgcgacgccgacaagcgcgccggcgtgcccgccggcaagctggtggagatcgacatcgccgtggacccctgcgagggcaccaacctgtgcgcctacggccagcccggcagcatggccgtgctggccatcagcgagaagggcggcctgttcgccgcccccgacttctacatgaagaagctggccgccccccccgccgccaagggcaaggtggacatcaacaagagcgccaccgagaacctgaagatcctgagcgagtgcctggaccgcgccatcgacgagctggtggtggtggtgatggaccgcccccgccacaaggagctgatccaggagatccgccaggccggcgcccgcgtgcgcctgatcagcgacggcgacgtgagcgccgccatcagctgcggcttcgccggcaccaacacccacgccctgatgggcatcggcgccgcccccgagggcgtgatcagcgccgccgccatgcgctgcctgggcggccacttccagggccagctgatctacgaccccgaggtggtgaagaccggcctgatcggcgagagccgcgagagcaacatcgcccgcctgcaggagatgggcatcaccgaccccgaccgcgtgtacgacgccaacgagctggccagcggccaggaggtgctgttcgccgcctgcggcatcacccccggcctgctgatggagggcgtgcgcttcttcaagggcggcgcccgcacccagagcctggtgatcagcagccagagccgcaccgcccgcttcgtggacaccgtgcacatgttcgacgacgtgaagaccgtgagcctgcgc

SEQ ID NO: 26 - protein, 516 aa, Apis mellifera
MSTISDNQCTSVRDFYKDRSIFITGGTGFMGKVLVEKLLRSCPGIKNIYILMRPKKSQDIQQRLQKLLDVPLFDKLRRDTPDELLKIIPIAGDVTEHELGISEADQNVIIRDVSIVFHSAATVKFDEPLKRSVHINMIGTKQLLNLCHRMHNLEALIHVSTAYCNCDRYDVAEEIYPVSAEPEEIMALTKLMDSQMIDNITPTLIGNRPNTYTFTKALTERMLQSECGHLPIAIVRPSIVLSSFREPVSGWVDNLNGPTGIVAAAGKGFFRSMLCQKNMVADLVPVDIVINLMICTAWRTATNRTKTIPIYHCCTGQQNPITWQQFVELILKYNRMHPPNDTIWWPDGKCHTFAIVNNVCKLFQHLLPAHILDFIFRLRGKPAIMVGLHEKIDKAVKCLEYFTMQQWNFRDDNVRQLSGELSPEDRQIFMFDVKQIDWPSYLEQYILGIRQFIIKDSPETLPAARSHIKKLYWIQKVVEFGMLLVVLRFLLLRIPMAQSACFTLLSAILRMCRMIV

SEQ ID NO: 27 - protein, 504 aa, Apis cerana cerana
MDKIKIVQSNNKENLKNTSDSQIQKFYTGKYIFFTGCTSILGSSILEKILISCTEISKIYIMIKLKNDILIKEQLKKYFQNEIFNTVRESNPNFMEKVVPIYGDLSKADLGLSSEDRRCLIENVNIIIHNGSIVQSTKVSYILRLNVIATQTLLELAMECSHLEAFVYVSTAFSHPYKQIIEEKFYPIYAGNIKIIEDVIRADEENESGITNEALRDIITDWVNLYIFSKAYAEDLVYNFGKKKSLPCVVFRPSMVVCTNEKLVPSKNKNGPVMLATAISLGYIHVSNLKKTDTMDLIPIDMTVNSLLAMIWDFVVYRKKEEPQQVYNYGSTDWNPITVDSASKMIFKEIEKNPSDNVIWKPYLIYIQNIYLFSILNILLNVIPNILIDLILLISKGEQPPIMRTIHKLKKHYFPFIQIFRSNQIIKTNKFKECLTRMNTTDLKEFSFNLATLNWNDSVVKLMTCCRKEMNEPITASPATKKKYQNLIEGKGLQNSTTPLLYIE

SEQ ID NO: 28 - protein, 517 aa, Apis dorsata
MDKIKIVQSDKENLKNTSDSQIQKFYTGKHIFFTGCTSFLGSSILEKILITCTEISKIYVMIKLKNDVLIKEQLKKYFQNEIFDTLRESNPNFIEKVVPIYGDLSKADLGLSSKNRRCLIENVNIIIHNGSIIQSPKASYILRLNVIATQTLLELATECSHLEAFVYVSTAFSHPYKQIIEEKFYPIAGNIKIIEDVIRADEENESGITNEALRNIMGDWVNLYAFSKAYAEDLVYNFGKTKSLPCVVFRPSMVVCTNEKLVPSKNKNGPVMLAMAISLGYIHVSNLKKTDTMDLIPIDMTANSLLAMIWDFVVYRKKEELQQVYNYGSTDWNPITVGSASEIIFKEVEKNPSNNVLWKPYLIYIQNIYLFSTLNILLNVIPGILIDLTLLICQEEPPIMRTIHKLKKHYLPFIQIFRPNQIIKTNKFKECLTRMNTTDLKEFSFNLATMNWNDNAVKLMTCCRKEMNEPTTASPATKKKYRNLVKLHFVICSLLIMLFLLYFFYRILSIFCHCYHH

SEQ ID NO: 29 - protein, 515 aa, Anas playrhynchos
MVSIPEYYEGKNVLLTGATGFMGKVLLEKLLRSCPKVQAVYVLVRHKSGQTPEARIQEITSCKLFDRLREEQPDFKEKIIVITSELTQPELDLSSPIKQKLIDCINIIFHCAATVRFNETLRDAVQLNVLSTKQLLSLAHQMTNLEVFIHVSTAYAYCNRKHIEEIVYPPPVDPKKLMDSLEWMDDGLVNDITPKLIGDRPNTYTYTKALAEYVVQQEGAKLNTAIIRPSIVGASWKEPFPGWIDNFNGPSGLFIAAGKGILRTMRATNGAVADLVPVDVVVNMTLAAAWYSGVNRPRNIMVYNCTTGGTNPFHWSEVEYHVISTFKRNPLEQAFRRPNVNLTSNHLLYHYWIAVSHKAPAFLYDIYLRITGRSPRMMKTISRLHKAMMLLEYFTSNSWIWNTENMTMLMNQLTPEDKKTFNFDVRQLHWAEYMENYCMGTKKYVLNEEMSGLPAARKHLNKLRNIRYGFNTILVILIWRIFIARSQMARNIWYFVVSLCYKFLSYFRASSTMRY

SEQ ID NO: 30 - protein, 515 aa, Canis lupus familiaris
MSMIAAFYSGKSILITGATGFMGKVLMEKLFRTSPDLKVIYILVRPKAGQTTQQRVFQILNSKLFEKVKEVCPNVHEKIRAIYADLNQNDFAISKEDMQELLSCTNIVFHCAATVRFDDHLRHAVQLNVTATQQLLLMASQMPKLEAFIHISTAFSNCNLKHIDEVIYPCPVEPKKIIDSMEWLDDAIIDEITPKLIGDRPNTYTYTKALGEMVVQQESGNLNIAIIRPSIVGATWQEPFPGWVDNLNGPSGLIIAAGKGFLRAIRATPMAVADLIPVDTVVNLTLAVGWYTAVHRPKSTLIYHCTSGNLNPCNWGKMGFQVLATFEKIPFERAFRRPYADFTTNTITTQYWNAVSHRAPAIIYDFYLRLTGRKPRMTKVMNRLLRTVSMLEYFVNRSWEWSTYNTEMLMSELSPEDQRVFNFDVRQLNWLEYIENYVLGVKKYLLKEDMAGIPEAKQHLKRLRNIHYLFNTALFLIAWRLLIARSQMARNVWFFIVSFCYKFLSYFRASSTLKV

SEQ ID NO: 31 - DNA, 1576 bp, Beta vulgaris subsp. vulgaris
tgtgtaatttctctaccaggggctaatagcctaatctatcaaaaagatttaagaatgcccgatctgaatccgacatgatttttgtttgtcgggaaatactatcaaattaaagcttgctgagcaaaatggaaattgatcactcctaattactattggtttttttaccgaaatgaaacaaagaatagagatattcctagcaactagcataaaaggtcaaccgtgaatcttggatttgtttctgcatcatataaagccttgcgagtatctgcttgtatatactagcaattaggcaattaactgagcacacaaacacaatcgagcagatagatcagcaaataggaaaagaatggagtctgagattaagaatttcatgaagatctggttattcgcaatttgttcagcttgttactccctgagtttatccagaatattccacatccgaagcggcattccaaggttactcttcatcctccccatcatctatctctttactgttctccctttatctctctcttcttttcatcttggtggtcccactatcttcttccttgtttggcttgctaattttaaacttcttctttacgcctttgatcttggtcctctttctactaatccaattacaaacaacaacaacaacaacaacaacaacgttaattccctatctctctctcatttcatttccattgctcttcttcccattaaagtcaatcaacaacaaccatcaaaacccacaaataataagtggaagtctgttctcatcattgccttcaaattactggcatttgctcttgtcatcaaaatctatgactttacccaacatttacccaaatttcttctattgattaattactgctgtcatctttaccttggtgttgaggtaactttagctgttgttgcagccatagttcgggccactttgggcttgggccttgacccacagtttaatgagccttatttggccacatcacttcaggatttttggggccgtagatggaatctgatggtgtcagacatcctacgcctctccgtttttaaccccatccgacgtgtcttctctccattggttggcaagaggtgggccctggtagttggaatgattgcggcatttactgtgtctggcctcatgcacgagctcatcttctattatttcacacgtgtgaaccccacgtgggaagtcacgtggttttttgtattacatgggatgtgtacggcggttgaagtggtggttaaggaggcagttggtggtcggttgcagttgcatcggttgatttcggggacgttgacgattgggtttgttgcagttacggcgtggtggctttttttacctcaaatcataagaaatggtgtggatgtcaaagtcattaatgagtatcctgtaatgtttagctttgtcaaacaacacatttttttttgtttcaaaaactgattcaattttcgattgtttctcactacaatagcatgctcagtcttggaatgctttcagtacaatagttcagtttttttattatctaagtagtttcttttatgatgtaatttttcatcttaatcataattcaacttggttgcttcatttcaa

SEQ ID NO: 32 - DNA, 1385 bp, Spinacia oleracea
ttaatggcgagtgaattcaagaagaatcagcaacagatttcttcgagaggctaaatggcggaacatggaggaggaccaccgcttcaccggcatcatttggaggtggttgccattacagaactgctataatttgaaccgttggattctgttgtagtggtggacctggtgactggtaaaccaatttaataatactgtataaaatctgttattccattaccaccaacaaaaactcacaaaaaataaccctaaaaaccaaaaatggagacagagatctggaatttcatcaagatatggggaatagcaatcgcttcagcctgctactcctactctttatccagaaccttccacatccaaaccggtattctccggttgttcttcatccttcccgtcatctacctcttcactgtcctcccactttctctctcctccttccatctcggtggtcccaccatcttctaccttgtttggcttgccaactttaaactactcctctactccttcaacctcggccctctttcttccaatccaaacacctccttatcgcatttcatcgccattgctcttctccccatcaaagtcaacgccggttcaacgccgaccaagaagcgggacccactcggatcacttcctctgtttgttgtaaaattactggcttttgcccttgtggtaaaagtttatgagtttcgccaagatttacctaaatcccttcttttgcttaattactgttgtcatctttaccttggtgtagaggttactttgggaattaccgcggccttggttcgggccagtttggggttgggcttggacccacagtttgatgagccgtacttggccacctcactccaggacttttggggccgtagatggaatctcatggtgtcggacatcttacggttgtccgtttacgaccccatccgacgtgttgtctcgccattggttgggaaaaggtgcggtttagtgggtgggatcgtaatgtcgtttactgtgtctgggctgatgcacgaggtgattttctattatttcacacgtgtgaggcccacgtgggaagtcacgtggttctttgttctacatggggtgtgcaccgcagtggaggtggtggttaagaaaatggttgtatcgaggtttcagttgcatcgattgatatcaggtccgttgacaattggattcataggggtcacagcatggtggttattcctccctcaaatcttaaggaatggtttcgacgtcaaagttataaatgagtatcctgtaatggttaattttgttaaggaaaatgttttgtacttttttattttgttggacaaacttttggtggcttgagcaattttgatttcgtctatgtagtcaactcggttatgatttatgaatgttatttttcaacttaa

SEQ ID NO: 33 - DNA, 1508 bp, Coffea Arabica
aaattggaatagtttcgtacgctcctttgctcattccagtccgcccgataaagcaagctctctcatcccaccgacgccacagaagccttgtttaacaagtggtcgccgtccgggggaattcgcaaaaccatttccaaatgggggacgagatcaagagttcaatctttgctttcgcatcggtgccagcatctctcagttactgctatttcattgccgcaagaatcccaaaagggttcttgaggctgatttttctcctacccgtctactatcacttcacaattctccctctttacatgcccattatcttctttagaggtgtctcaacactcttcataacatggctcggcaacttcaagctgctcctctttgcctttggacgaggtccactctcctcggaccaatccatgcccttgcacatcttcatcgcctcctgtgctctccccatcagaaccaagctgccaaatgtcaacccctcatctacttcttcccgaccctccaagaaaaagccatggtttttaaatttaggaacggagatcttagctttattctctttatttgggctggcagccaaatatgaagaaactgtacaccccgtagttgtacaaatagcctatagttgcgcgatgtttttcctaattgaagttctggtgacgctctctagttccgcggtccgagccctggtgggtctagagctggaggcaccgtccgacgagccctacttatcagcttctctgcaagatttctggggcaagaggtggaacctctcagtaacaaatgcactgcggcacacaatatacaagcccgtcaggtcaatatcggcggtcgtactggggaatcgatcagccgcactgcctgccatcttcgcgacctttcttgtctctggtctaatgcatgaactcatatactattacctctcaggtgtgaagccctcctgggaagtgacgtggttcttcgttctgcatggaatttgtgttgtgattgaaatggtgttgaagacagctttgggaggaaaatgggcggtgccccggttaattgcggccccgttgacacttgggtttgtgatttcaaccggtatgtggttgtttttccctccgttgaccgagatggggattgataaaatggtttttgaagagttcagttgcgctggcgagtatgtgaagggtaggctggtggccttatgtcccactatcctgggccacaaatcgaggagttaagactcagtcgggctcgggcagtctgaaaacgacgacgggccatcaagaaatgtctcccacatttccgtcctaataaaatggacaactgtttgtccgtagttgactttaaagttcaattatgcatgcgtgtggtccccttctagcgttcaatttcgggattatatatctcatctcagttgtaatattattgtcgcttcctcgtcacaatcagagactggatgctgcgacttttcgcgtgctttctgcaattcaagagccggtttggttttgggttgttatcaaaatatattagta

SEQ ID NO: 34 - DNA, 1032 bp, Cuscuta australis isolate Yunnan
atggagaagatctcactaacccacgtctggtttctggttttggcttctctggtgtactgctatttcgtgtctgcaaacctcccaaagggcattttcaggttcatatctctaacccctgttttcggcctcttcgctgtcttccctctcctccactcctccgccttctgcacggcggtcgccttcttcttcttcacctggctctccaacttcaagctcctcgccttctccttcgaccgcggcccgctctcctcctcctcacccgcctacaggtctctcctcaccttcattgccatggcttctcttcctctcaggttgaagaagaaaaatgtcaatagatcaaaggtacagattttgcggttaaacttggcggcggaaattgcgggcttcgcggggttgttgcagctgattttccggtacggagatggggcccaccagaacctggtcttgatctggtattctctcctggttttcctcatggtggatgtgctggtcggagtttcgggattcgcggtccgggtcttgaccggtctagatctggacccgccgtcggacgagccttacctctcctgctccctccgggaattctgggggaggcgctggaacctcaccgtgaccaacaccttccgcttctccgtctacgatcccgtccgggaactctccgccgccgtcatcggcggcgcgtgggccccacttccggcgatgatggcgacgttcgcgctctccggcctcatgcacgagctgctggtcttctacgtcgcgcgcgcccgcccgtcgtgggagatgacggcgttcttcttgctccacggagtctgcgtcgcggcggagtacgcgacggagcaggcttggggaggcactccccggctgccgcgggcggtttcggggccgttgacggtcgggttcgtggtgggcaccaccttctggctgttcttcccgccgctaattaggagcggcgccgacaaaatggtcctggaagaattgaaaacctatatccagttcattcataaccattggagatcattagtgattgcgaat

SEQ ID NO: 35 - protein, 359 aa, Beta vulgaris subsp. vulgaris
MESEIKNFMKIWLFAICSACYSLSLSRIFHIRSGIPRLLFILPIIYLFTVLPLSLSSFHLGGPTIFFLVWLANFKLLLYAFDLGPLSTNPITNNNNNNNNNVNSLSLSHFISIALLPIKVNQQQPSKPTNNKWKSVLIIAFKLLAFALVIKIYDFTQHLPKFLLLINYCCHLYLGVEVTLAVVAAIVRATLGLGLDPQFNEPYLATSLQDFWGRRWNLMVSDILRLSVFNPIRRVFSPLVGKRWALVVGMIAAFTVSGLMHELIFYYFTRVNPTWEVTWFFVLHGMCTAVEVVVKEAVGGRLQLHRLISGTLTIGFVAVTAWWLFLPQIIRNGVDVKVINEYPVMFSFVKQHIFFCFKN

SEQ ID NO: 36 - protein, 353 aa, Spinacia oleracea
METEIWNFIKIWGIAIASACYSYSLSRTFHIQTGILRLFFILPVIYLFTVLPLSLSSFHLGGPTIFYLVWLANFKLLLYSFNLGPLSSNPNTSLSHFIAIALLPIKVNAGSTPTKKRDPLGSLPLFVVKLLAFALVVKVYEFRQDLPKSLLLLNYCCHLYLGVEVTLGITAALVRASLGLGLDPQFDEPYLATSLQDFWGRRWNLMVSDILRLSVYDPIRRVVSPLVGKRCGLVGGIVMSFTVSGLMHEVIFYYFTRVRPTWEVTWFFVLHGVCTAVEVVVKKMVVSRFQLHRLISGPLTIGFIGVTAWWLFLPQILRNGFDVKVINEYPVMVNFVKENVLYFFILLDKLLVA

SEQ ID NO: 37 - protein, 358 aa, Coffea Arabica
MGDEIKSSIFAFASVPASLSYCYFIAARIPKGFLRLIFLLPVYYHFTILPLYMPIIFFRGVSTLFITWLGNFKLLLFAFGRGPLSSDQSMPLHIFIASCALPIRTKLPNVNPSSTSSRPSKKKPWFLNLGTEILALFSLFGLAAKYEETVHPVVVQIAYSCAMFFLIEVLVTLSSSAVRALVGLELEAPSDEPYLSASLQDFWGKRWNLSVTNALRHTIYKPVRSISAVVLGNRSAALPAIFATFLVSGLMHELIYYYLSGVKPSWEVTWFFVLHGICVVIEMVLKTALGGKWAVPRLIAAPLTLGFVISTGMWLFFPPLTEMGIDKMVFEEFSCAGEYVKGRLVALCPTILGHKSRS

SEQ ID NO: 38 - protein, 342 aa, Cuscuta australis
MEKISLTHVWFLVLASLVYCYFVSANLPKGIFRFISLTPVFGLFAVFPLLHSSAFCTAVAFFFFTWLSNFKLLAFSFDRGPLSSSSPAYRSLLTFIAMASLPLRLKKKNVNRSKILRLNLAAEIAGFAGLLQLIFRYGDGAHQNLVLIWYSLLVFLMVDVLVGVSGFAVRVLTGLDLDPPSDEPYLSCSLREFWGRRWNLTVTNTFRFSVYDPVRELSAAVIGGAWAPLPAMMATFALSGLMHELLVFYVARARPSWEMTAFFLLHGVCVAAEYATEQAWGGTPRLPRAVSGPLTVGFVVGTTFWLFFPPLIRSGADKMVLEELKTYIQFIHNHWRSLVIAN

SEQ ID NO: 39 - protein, 390 aa, Homo saipan
MRKMLAAVSRVLSGASQKPASRVLVASRNFANDATFEIKKCDLHRLEEGPPVTTVLTREDGLKYYRMMQTVRRMELKADQLYKQKIIRGFCHLCDGQEACCVGLEAGINPTDHLITAYRAHGFTFTRGLSVREILAELTGRKGGCAKGKGGSMHMYAKNFYGGNGIVGAQVPLGAGIALACKYNGKDEVCLTLYGDGAANQGQIFEAYNMAALWKLPCIFICENNRYGMGTSVERAAASTDYYKRGDFIPGLRVDGMDILCVREATRFAAAYCRSGKGPILMELQTYRYHGHSMSDPGVSYRTREEIQEVRSKSDPIMLLKDRMVNSNLASVEELKEIDVEVRKEIEDAAQFATADPEPPLEELGYHIYSSDPPFEVRGANQWIKFKSVS

SEQ ID NO: 40 - protein, 359 aa, Homo sapiens
MAAVSGLVRRPLREVSGLLKRRFHWTAPAALQVTVRDAINQGMDEELERDEKVFLLGEEVAQYDGAYKVSRGLWKKYGDKRIIDTPISEMGFAGIAVGAAMAGLRPICEFMTFNFSMQAIDQVINSAAKTYYMSGGLQPVPIVFRGPNGASAGVAAQHSQCFAAWYGHCPGLKVVSPWNSEDAKGLIKSAIRDNNPVVVLENELMYGVPFEFPPEAQSKDFLIPIGKAKIERQGTHITVVSHSRPVGHCLEAAAVLSKEGVECEVINMRTIRPMDMETIEASVMKTNHLVTVEGGWPQFGVGAEICARIMEGPAFNFLDAPAVRVTGADVPMPYAKILEDNSIPQVKDIIFAIKKTLNI

SEQ ID NO: 41 - protein, 390 aa, Mus musculus
MRKMLAAVSRVLAGSAQKPASRVLVASRNFANDATFEIKKCDLHRLEEGPPVTTVLTREDGLKYYRMMQTVRRMELKADQLYKQKIIRGFCHLCDGQEACCVGLEAGINPTDHLITAYRAHGFTFTRGLPVRAILAELTGRRGGCAKGKGGSMHMYAKNFYGGNGIVGAQVPLGAGIALACKYNGKDEVCLTLYGDGAANQGQIFEAYNMAALWKLPCIFICENNRYGMGTSVERAAASTDYYKRGDFIPGLRVDGMDILCVREATKFAAAYCRSGKGPILMELQTYRYHGHSMSDPGVSYRTREEIQEVRSKSDPIMLLKDRMVNSNLASVEELKEIDVEVRKEIEDAAQFATADPEPPLEELGYHIYSSDPPFEVRGANQWIKFKSVS

SEQ ID NO: 42 - protein, 359 aa, Mus musculus
MAVVAGLVRGPLRQASGLLKRRFHRSAPAAVQLTVREAINQGMDEELERDEKVFLLGEEVAQYDGAYKVSRGLWKKYGDKRIIDTPISEMGFAGIAVGAAMAGLRPICEFMTFNFSMQAIDQVINSAAKTYYMSAGLQPVPIVFRGPNGASAGVAAQHSQCFAAWYGHCPGLKVVSPWNSEDAKGLIKSAIRDNNPVVMLENELMYGVAFELPAEAQSKDFLIPIGKAKIERQGTHITVVAHSRPVGHCLEAAAVLSKEGIECEVINLRTIRPMDIEAIEASVMKTNHLVTVEGGWPQFGVGAEICARIMEGPAFNFLDAPAVRVTGADVPMPYAKVLEDNSVPQVKDIIFAVKKTLNI

SEQ ID NO: 43 - protein, 420 aa, Saccharomyces cerevisiae
MLAASFKRQPSQLVRGLGAVLRTPTRIGHVRTMATLKTTDKKAPEDIEGSDTVQIELPESSFESYMLEPPDLSYETSKATLLQMYKDMVIIRRMEMACDALYKAKKIRGFCHLSVGQEAIAVGIENAITKLDSIITSYRCHGFTFMRGASVKAVLAELMGRRAGVSYGKGGSMHLYAPGFYGGNGIVGAQVPLGAGLAFAHQYKNEDACSFTLYGDGASNQGQVFESFNMAKLWNLPVVFCCENNKYGMGTAASRSSAMTEYFKRGQYIPGLKVNGMDILAVYQASKFAKDWCLSGKGPLVLEYETYRYGGHSMSDPGTTYRTRDEIQHMRSKNDPIAGLKMHLIDLGIATEAEVKAYDKSARKYVDEQVELADAAPPPEAKLSILFEDVYVKGTETPTLRGRIPEDTWDFKKQGFASRD

SEQ ID NO: 44 - protein, 366 aa, Saccharomyces cerevisiae
MFSRLPTSLARNVARRAPTSFVRPSAAAAALRFSSTKTMTVREALNSAMAEELDRDDDVFLIGEEVAQYNGAYKVSKGLLDRFGERRVVDTPITEYGFTGLAVGAALKGLKPIVEFMSFNFSMQAIDHVVNSAAKTHYMSGGTQKCQMVFRGPNGAAVGVGAQHSQDFSPWYGSIPGLKVLVPYSAEDARGLLKAAIRDPNPVVFLENELLYGESFEISEEALSPDFTLPYKAKIEREGTDISIVTYTRNVQFSLEAAEILQKKYGVSAEVINLRSIRPLDTEAIIKTVKKTNHLITVESTFPSFGVGAEIVAQVMESEAFDYLDAPIQRVTGADVPTPYAKELEDFAFPDTPTIVKAVKEVLSIE

(19) United States
(12) Patent Application Publication
(10) Pub. No.: US 2021/0230652 A1
(43) Pub. Date: Jul. 29, 2021

(54) SYSTEMS, METHODS AND COMPOSITIONS FOR THE GENERATION NOVEL HIGH YIELDING WAXES FROM MICROALGAE

(71) Applicant: Pebble Labs USA Inc., Santa Fe, NM (US)

(72) Inventors: Richard Sayre, Los Alamos, NM (US); Tawanda Zidenga, White Rock, NM (US); Stephanie Willette, Santa Fe, NM (US)

(21) Appl. No.: 17/048,923

(22) PCT Filed: Apr. 18, 2019
(86) PCT No.: PCT/US19/28213

§ 371 Date: Oct. 19, 2020

Related U.S. Application Data
(60) Provisional application No. 62/659,282, filed on Apr. 18, 2018.

(51) Int. Cl. C12P 7/64; C12N 1/12; C12N 9/10; C12N 9/02; C12N 9/16

(52) U.S. Cl. CPC C12P 7/6436; C12Y 102/01084; C12Y 203/01075; C12N 1/12; C12Y 203/0102; C12N 2500/02; C12Y 102/04001; C12N 9/0008; C12Y 301/03037; C12N 9/16; C12N 2500/30; C12N 9/1029

(57) ABSTRACT

The invention relates to the chemical synthesis of waxes. Specifically, the invention relates to systems and methods for the high-yield production of novel and high-value waxes in genetically-modified algae-based systems as a replacement for petroleum-based products.

This application claims the benefit of and priority to U.S. Provisional Patent Application No. 62/659,282, filed Apr. 18, 2018, which is incorporated herein by reference in its entirety.

### SEQUENCE LISTING

The instant application contains a Sequence Listing which has been submitted electronically in ASCII format and is hereby incorporated by reference in its entirety.

### TECHNICAL FIELD

The invention relates to the chemical synthesis of waxes. Specifically, the invention relates to systems and methods for the production of novel and high-value waxes in genetically-modified algae-based systems as a replacement for petroleum-based products.

### BACKGROUND

Increased demand for energy by the global economy, as well as concerns related to global climate change and other environmental factors, has triggered the need for environmentally sustainable alternatives to petroleum-based industrial products. One such petroleum-based product that is seeing increasing global demand are waxes. As generally shown in FIG. 1, waxes have the general chemical formula CH₃(CH₂)—CHO where “n” is typically >20, and have melting temperatures >37° C. Waxes are used in many industrial applications including: candles, paints, Coatings, barriers, resins, plastics, synthetic rubber manufacturing, tire manufacturing, polish, sanitation goods, corrugated and solid fiber box Coatings, and printing ink manufacturing. The projected growth demand in the global wax market is $9 Billion in revenue and 500,000 tons annually by 2020. US wax demands are expected to grow to more than 3 billion pounds by 2019, with a value of $3.2 billion dollars. Crude petroleum provides up to 97% of the wax consumed in the US largely as paraffin. In addition, the United States imports a significant share (65%) of the global wax market. Increased pressure from governments, environmental organizations, and the public, however, continues to drive the need for renewable solutions to petroleum-based waxes. For example, in 2021, the European Union has legislated a ban on single use plastics creating a greater demand for water impermeable sustainable Coatings such as green waxes for packaging.

There is a premium for “green waxes” e.g. bees wax, which substantially reduce carbon emissions. Some organisms can accumulate large quantities of wax esters. Sperm whales are the source of sperm whale oil which contains up to 95% of wax esters consisting of 34 carbons. With the banning of whale hunting in 1987, sperm whale oil is no longer legally sold. The best alternative to whale oil is now jojoba oil, produced from the seed of the desert shrub jojoba (Simmondsia chinensis). In contrast to all known oil storing plant seeds, which store triacylglycerols (TAGs), jojoba stores wax esters in its seeds. Wax esters (C38 to C44) account for up to 60% of the dry weight of the cotyledons of jojoba and are composed of very long-chain (C20, C22, and C24) monounsaturated fatty acids and alcohols (FIG. 1B).

The challenge for green waxes has been, however, that their prices are substantially greater than paraffin. For example, as highlighted in FIG. 1A, one of the more valuable bio-based waxes is bees wax (BW). Chemically, bees wax is made up of C30 esters. Paraffin has similar C30 units but lacks the ester bond linking the C30 acyl chains. Currently, BW sells for $3.20/lb. depending on quality and is largely imported from India. In contrast, paraffin is sold for $0.88/lb.

Select species of algae produce a class of energy-dense hydrocarbons waxes in contrast to the vast majority which store reducing power as oils. For example, a variety of algae have been shown to accumulate waxes ranging in yields from <1% (dry weight) to as high as 70%. The algae Euglena gracilis also referred to as E. gracilis or Euglena, is one of several species that accumulate wax esters. In some Euglena strains, waxes accumulate up to 30% of the total dry weight (dw) when grown (<24 hrs.) under anaerobic conditions and as high as 60% (dw) when grown anaerobically in the presence of fatty acid elongase inhibitors or in the presence of bicarbonate. More specifically, wax synthesis in Euglena is facultatively induced by various stresses including: anaerobiosis and heat stress. Under anaerobic conditions, the source of carbon skeletons (acetyl-CoA) for fatty acid synthesis and wax production in Euglena is paramylon starch. Importantly, the production of fatty acids and ultimately wax from paramylon allows the cell to turnover NADH in the absence of oxygen. Thus, wax synthesis serves as a means to store reducing equivalents under anaerobic conditions. Those reducing equivalents can be recovered once the algae are returned to air. Under aerobic conditions waxes are oxidized providing reducing power for ATP production via respiration.

Euglena facultatively produces C20 to C36 wax esters comprised of saturated fatty acids and alcohols of 12-18 carbon chains with myristyl myristate (14:0-14:0) as the major species. The carbon chain length of the dominant acyl ester in Euglena waxes ranges from C25-C30, which is nearly identical to bees wax (C30) and petroleum-derived paraffin (C30). With the global market for bees wax currently exceeding $93.3 million/year, and given that bees wax costs 3.6× more than paraffin, the development of a less expensive bio-based bees wax alternative would allow for the replacement of paraffin in many applications. Recently, Euglena waxes have been shown to substitute for paraffin in tire manufacturing. In 2014, U.S. Pat. No. 8,664,312, teaches the use of renewable Euglena wax in the manufacturing of tires. The addition of 1-10% (dw) Euglena wax to tires substantially increased resistance to weathering (5× increase in ozone resistance) and resistance to abrasion.

In addition to tire manufacturing, natural waxes can also substitute for paraffin in a variety of other applications including: lubricants, emollients, insulators, Coatings and adhesives, inks, PVC lubrication, and potentially novel applications. Given that the integrated capital and operating expenses for producing algal biomass is estimated to be $500/ton and given a demonstrated wax yield from Euglena is ˜50%, the minimum estimated cost for producing wax from Euglena is $0.50/lb or 43% less than petroleum-based paraffin. Thus, the economics for producing natural wax substitutes for paraffin in algae may have significant previously unrealized economic benefits. Unfortunately, Euglena is not the best production algae for waxes. Euglena lacks a cell wall and thus is much more susceptible to mechanical damage and pathogen attack than more robust alga species such as Chlorella. Chlorella species are among the highest biomass producing algae but store reducing equivalents as triacylglycerols and not as waxes. Significantly, since Chlorella does not produce wax it is anticipated that it cannot metabolize waxes. Thus Chlorella has the potential to be a more efficient wax producing and accumulating species for industrial production.

As described below, by overexpressing genes for wax biosynthesis from Euglena and jojoba in Chlamydomonas reinhardtii, the present have generated engineered waxes from algae to replace petroleum-based waxes, sperm whale waxes, bees waxes and jojoba waxes. Producing these in algae rather than plants represents a more cost-effective system of wax production due to the high potential levels of wax accumulation (≤50% dry weight). In addition, the present inventors have also demonstrated that feeding a fatty alcohol to these transgenic lines changes the profile of wax esters produced.

### SUMMARY OF THE INVENTION

One aim of the current invention may include the generation of one or more genetically engineered algae that produce one or more novel waxes. Such bio-engineered waxes may replace traditional petroleum-based waxes. Additional aims of the current invention may include the generation of one or more genetically engineered algae that produce one or more novel waxes at higher than wild type levels. Additional aims of the current invention may include the generation of one or more genetically engineered algae that produce one or more wax esters at higher than wild type levels.

An additional aim of the current invention may include the generation of transgenic, high biomass producing algae that typically do not synthesize or metabolize waxes and those overexpress genes that are involved in the bio-synthetic production of wax under the control of inducible gene promoters such as nitrate reductase. Another aim of the current invention may include the generation of transgenic algae, such as Chlamydomonas, that repress or under express certain genes that may result in the diversion of fatty acids toward the biosynthesis pathways of wax production.

Another aim of the current invention may include the dsRNA gene-silencing of certain genes in algae that may result in enhanced production of wax production. Another aim of the current invention may include the incorporation of a large-scale commercial system to grow sufficient quantities of algae to generate commercial quantities of wax.

Another aim may be the use of inducible gene promoters to turn on wax accumulation and maximize yield prior to harvesting and or reaching potentially toxic levels in the algae

An additional aim of the current invention may include the generation of novel wax compounds having commercially beneficial properties. Additional aims of the invention may further include the biosynthesis of novel waxes having commercially beneficial properties in wild-type and engineered algae by feeding the algae un-natural free acyl alcohols.

The invention may include the generation of transgenic algae strains that include enhanced production of waxes. In one embodiment, the invention may include the generation of a transgenic strain of microalgae that may overexpress fatty acyl-CoA reductase (FAR) and wax synthase (WS) genes. In this preferred embodiment, FAR and/or WS genes from one or more plant species such as Simmondsia chinensis (Jojoba), sorghum, Arabidopsis, palm tree (Copernicia prunifera) and other identified in the Sequence Listing. may be heterologously expressed in fast growing microalgae. In this embodiment, the FAR and/or WS genes may be part of an expression vector and may further be operably linked to one or more promoters.

In another embodiment of the invention, one or more strains of algae may be genetically modified to divert fatty acids to wax biosynthetic pathways. In one preferred embodiment, triacylglycerol (TAG) synthesis may be suppressed in fast growing microalgae to divert fatty acids to wax production. In this embodiment, diacylglycerol acyl transferase (DGAT2), and/or related gene family members in algae involved in TAG production may be transiently suppressed. In a preferred embodiment, this suppression may be through the production of dsRNA configured to target diacylglycerol acyl transferase (DGAT2), and/or related gene family members in algae involved in TAG production regulated by inducible gene promoters (e.g., nitrate reductase). In certain embodiments, production of such dsRNA may be operably linked to one or more promoters.

In another embodiment of the invention, one or more strains of algae may be genetically modified to suppress the activity of fatty acid elongase (FAE) activity in algae. In one preferred embodiment, very long chain fatty acid (VLCFA) elongases may be suppressed in fast growing microalgae. In a preferred embodiment, this suppression may be through the production of dsRNA configured to target fatty acid elongases (FAE), and/or related gene family members in algae involved in fatty acid, or very long chain fatty acid (VLCFA) production. In certain embodiments, production for such dsRNA may be operably linked to one or more promoters.

In yet another embodiment, the current invention may include the chemical synthesis of novel long chain and/or branched acyl alcohols that may be fed to transgenic algae expressing one or more heterologous wax synthase (WS) genes from various organisms, such as plants listed above. In this embodiment, such synthetic acyl alcohols may be incorporated into wax biosynthetic pathways to produce novel waxes having commercially beneficial properties, for example waxes that may be more similar to high value carnauba wax. Additional embodiments may include the generation of novel waxes with unique physical properties in wild-type and engineered algae by feeding the algae un-natural synthetic free acyl alcohols.

In yet another embodiment the levels of acetyl-CoA production for enhanced fatty acid production may be enhanced by elevating pyruvate dehydrogenase levels.

Additional aspects of the invention may include:

1. A method of the wax biosynthesis comprising the step of transforming an algal cell with one or more polynucleotide sequences operably linked to a promoter that expresses a heterologous fatty acyl-CoA reductase (FAR), and a heterologous wax synthase (WS) wherein said FAR and WS peptides operate to biosynthesize wax esters.
2. The method of embodiment 1 wherein said step of transforming comprises the step of transforming a Chlamydomonas reinhardtii cell.
3. The method of embodiment 1 wherein said promoter comprises an inducible promoter selected from the group consisting of: a nitrate-inducible NIT1 promoter, and copper-inducible CYC6 promoter.
4. The method of embodiment 1 wherein said heterologous fatty acyl-CoA reductase (FAR) is selected from the group consisting of:
a heterologous fatty acyl-CoA reductase (FAR) from Simmondsia chinensis; and
a heterologous fatty acyl-CoA reductase (FAR) from Euglena gracilis.
   5. The method of embodiment 4 wherein said heterologous fatty acyl-CoA reductase (FAR) is selected from the group consisting of:
a heterologous fatty acyl-CoA reductase (FAR) according to amino acid sequence SEQ ID NO. 1; and
a heterologous fatty acyl-CoA reductase (FAR) according to amino acid sequence SEQ ID NO. 5.
   6. The method of embodiment 1 wherein said heterologous wax synthase (WS) is selected from the group consisting of:
a heterologous wax synthase (WS) from Simmondsia chinensis; and
a heterologous wax synthase (WS) from Euglena gracilis.
   7. The method of embodiment 6 wherein said heterologous wax synthase (WS) is selected from the group consisting of:
a heterologous wax synthase (WS) according to amino acid sequence SEQ ID NO. 2; and
a heterologous wax synthase (WS) according to amino acid sequence SEQ ID NO. 6;
   8. The method of embodiments 4 and 6 wherein said biosynthesized wax ester comprises a C42:1 wax ester.
   9. The method of embodiments 5 and 7 and further comprising the step of producing an acyl species having an identity of C20:1/C22:0.
   10. The method of embodiments 5 and 7 wherein said biosynthesized wax ester comprises a C42:1 wax ester.
   11. The method of embodiment 10 and further comprising the step of culturing the transformed algal cell and feeding said algal culture a quantity of 1-dodecanol.
   12. The method of embodiment 11 and further comprising the step of biosynthesizing a C34:2 wax ester after feeding said algal culture a quantity of 1-dodecanol.
   13. The method of embodiment 11 and further comprising the step of producing hydroxylated triacylglycerol species (ETAG, OHTAG) in said algal culture after feeding said algal culture a quantity of 1-dodecanol.
   14. The method of embodiment 1 wherein said heterologous wax synthase (WS) that biosynthesizes wax esters from said acyl alcohol comprises a heterologous acyl-CoA:diacylglycerol acyltransferase that biosynthesizes wax esters from said acyl alcohol.
   15. The method of embodiment 14 wherein said acyl-CoA:diacylglycerol acyltransferase (DGAT) comprises a acyl-CoA:diacylglycerol acyltransferase from Euglena gracilis according to the amino acid sequence selected from the group consisting of: SEQ ID NO. 9, SEQ ID NO. 11, and 13.
   16. The method of embodiment 1 and further comprising the step of downregulating the expression of diacylglycerol acyl transferase (DGAT2) in said transformed algal cell.
   17. The method of embodiment 16 wherein said step of downregulating the expression of diacylglycerol acyl transferase (DGAT2) in said transformed algal cell comprises the step of transforming said algal cell to express a double-stranded RNA (dsRNA) configured to initiate an RNA-interference mechanism directed to expression of diacylglycerol acyl transferase (DGAT2).
   18. The method of embodiment 1 and further comprising the step of downregulating the expression of very long chain fatty acid (VLCFA) elongases in said transformed algal cell.
   19. The method of embodiment 18 wherein said step of downregulating the expression of at least one very long chain fatty acid (VLCFA) elongase in said transformed algal cell comprises the step of transforming said algal cell to express a double-stranded RNA (dsRNA) configured to initiate an RNA-interference mechanism directed to expression of at least one long chain fatty acid (VLCFA) elongase.
   20. The method of embodiment 1 and further comprising the step of downregulating the expression of fatty acid elongase (FAE).
   21. The method of embodiment 20 wherein said step of downregulating the expression of fatty acid elongase (FAE) comprises the step of transforming said algal cell to express a double-stranded RNA (dsRNA) configured to initiate an RNA-interference mechanism directed to expression of fatty acid elongase (FAE).
   22. The method of embodiment 1 and further comprising the step of increasing expression of pyruvate dehydrogenase in said transformed algal cell to increase production of acetyl-CoA.
   23. The method of embodiment 22 wherein said step of increasing expression of pyruvate dehydrogenase in said transformed algal cell to increase production of acetyl-CoA comprises the step of transforming said algal cell to express a heterologous pyruvate dehydrogenase complex.
   24. The method of embodiment 23 wherein said step of transforming said algal cell to express a heterologous pyruvate dehydrogenase complex comprises the step of transforming said algal cell to express a heterologous pyruvate dehydrogenase complex selected from the group of amino acid sequences SEQ ID NOs. 38-43.
   25. The method of embodiment 1 and further comprising the step of transforming said algal cell to express a heterologous cyanobacterial fructose-1,6-/sedoheptulose-1,7-bisphosphatase (fII).
   26. The method of embodiment 1 wherein said step of transforming said algal cell to express a heterologous cyanobacterial fructose-1,6-/sedoheptulose-1,7-bisphosphatase (fII) comprises the step of transforming said algal cell to express a heterologous cyanobacterial fructose-1,6-/sedoheptulose-1,7-bisphosphatase (fII) according to amino acid sequence SEQ ID NO. 24.
   27. The method of embodiment 1 and further comprising the step of culturing the transformed algal cell under low nitrogen conditions.
   28. A method of novel wax biosynthesis in algae comprising the steps of:
transforming an algal cell with one or more polynucleotide sequences operably linked to a promoter that expresses:
   a heterologous fatty acyl-CoA reductase (FAR) peptide that reduces long-chain-fatty-acyl-CoA to acyl alcohol;
   a heterologous wax synthase (WS) peptide that biosynthesizes wax esters from said acyl alcohol;
culturing said algal cell; and
harvesting the biosynthesized wax esters from the algal cell culture.
   29. A method of novel wax biosynthesis in algae comprising the steps of:
transforming an algal cell with one or more polynucleotide sequences operably linked to a promoter that expresses:
   a heterologous fatty acyl-CoA reductase from Simmondsia chinensis according to amino acid sequence SEQ ID NO. 1, that reduces long-chain-fatty-acyl-CoA to acyl alcohol;
   a heterologous wax synthase from Simmondsia chinensis according to amino acid sequence SEQ ID NO. 2, that biosynthesizes wax esters from said acyl alcohol;
culturing said algal cell; and
harvesting the biosynthesized wax esters from the algal cell culture.
   30. The method of embodiment 29 wherein said step of transforming comprises the step of transforming a Chlamydomonas reinhardtii cell.
   31. The method of embodiment 29 wherein said promoter comprises an inducible promoter selected from the group consisting of: a nitrate-inducible NIT1 promoter, and copper-inducible CYC6 promoter.
   32. The method of embodiments 29 wherein said biosynthesized wax ester comprises a C42:1 wax ester.
   33. The method of embodiments 29 and further comprising the step of producing an acyl species having an identity of C20:1/C22:0.
   34. The method of embodiment 29 and further comprising the step of culturing the transformed algal cell and feeding said algal culture a quantity of 1-dodecanol.
   35. The method of embodiment 34 and further comprising the step of biosynthesizing a C34:2 wax ester after feeding said algal culture a quantity of 1-dodecanol.
   36. The method of embodiment 24 and further comprising the step of producing hydroxylated triacylglycerol species (ETAG, OHTAG) in said algal culture after feeding said algal culture a quantity of 1-dodecanol.
   37. The method of embodiment 29 and further comprising the step of downregulating the expression of diacylglycerol acyl transferase (DGAT2) in said transformed algal cell.
   38. The method of embodiment 37 wherein said step of downregulating the expression of diacylglycerol acyl transferase (DGAT2) in said transformed algal cell comprises the step of transforming said algal cell to express a double-stranded RNA (dsRNA) configured to initiate an RNA-interference mechanism directed to expression of diacylglycerol acyl transferase (DGAT2).
   39. The method of embodiment 29 and further comprising the step of downregulating the expression of very long chain fatty acid (VLCFA) elongases in said transformed algal cell.
   40. The method of embodiment 39 wherein said step of downregulating the expression of at least one very long chain fatty acid (VLCFA) elongase in said transformed algal cell comprises the step of transforming said algal cell to express a double-stranded RNA (dsRNA) configured to initiate an RNA-interference mechanism directed to expression of at least one long chain fatty acid (VLCFA) elongase.
   41. The method of embodiment 29 and further comprising the step of downregulating the expression of fatty acid elongase (FAE).
   42. The method of embodiment 41 wherein said step of downregulating the expression of fatty acid elongase (FAE) comprises the step of transforming said algal cell to express a double-stranded RNA (dsRNA) configured to initiate an RNA-interference mechanism directed to expression of fatty acid elongase (FAE).
   43. The method of embodiment 29 and further comprising the step of increasing expression of pyruvate dehydrogenase in said transformed algal cell to increase production of acetyl-CoA.
   44. The method of embodiment 43 wherein said step of increasing expression of pyruvate dehydrogenase in said transformed algal cell to increase production of acetyl-CoA comprises the step of transforming said algal cell to express a heterologous pyruvate dehydrogenase complex.
   45. The method of embodiment 44 wherein said step of transforming said algal cell to express a heterologous pyruvate dehydrogenase complex comprises the step of transforming said algal cell to express a heterologous pyruvate dehydrogenase complex selected from the group of amino acid sequences SEQ ID NOs. 38-43.
   46. The method of embodiment 29 and further comprising the step of transforming said algal cell to express a heterologous cyanobacterial fructose-1,6-/sedoheptulose-1,7-bisphosphatase (fII).
   47. The method of embodiment 29 wherein said step of transforming said algal cell to express a heterologous cyanobacterial fructose-1,6-/sedoheptulose-1,7-bisphosphatase (fII) comprises the step of transforming said algal cell to express a heterologous cyanobacterial fructose-1,6-/sedoheptulose-1,7-bisphosphatase (fII) according to amino acid sequence SEQ ID NO. 24.
   48. The method of embodiment 29 and further comprising the step of culturing the transformed algal cell under low nitrogen conditions.
   49. A method of novel wax biosynthesis in algae comprising the steps of:
transforming an algal cell with one or more polynucleotide sequences operably linked to a promoter that expresses:
   a heterologous fatty acyl-CoA reductase from Euglena gracilis according to amino acid sequence SEQ ID NO. 5, that reduces long-chain-fatty-acyl-CoA to acyl alcohol; and
   a heterologous wax synthase from Euglena gracilis according to amino acid sequence SEQ ID NO. 6, that biosynthesizes wax esters from said acyl alcohol;
culturing said algal cell; and
harvesting the biosynthesized wax esters from the algal cell culture.
   50. The method of embodiment 49 wherein said step of transforming comprises the step of transforming a Chlamydomonas reinhardtii cell.
   51. The method of embodiment 49 wherein said promoter comprises an inducible promoter selected from the group consisting of: a nitrate-inducible NIT1 promoter, and copper-inducible CYC6 promoter.
   52. The method of embodiments 49 wherein said biosynthesized wax ester comprises a C42:1 wax ester.
   53. The method of embodiments 49 and further comprising the step of producing an acyl species having an identity of C20:1/C22:0.
   54. The method of embodiment 49 and further comprising the step of culturing the transformed algal cell and feeding said algal culture a quantity of 1-dodecanol.
   55. The method of embodiment 54 and further comprising the step of biosynthesizing a C34:2 wax ester after feeding said algal culture a quantity of 1-dodecanol.
   56. The method of embodiment 54 and further comprising the step of producing hydroxylated triacylglycerol species (ETAG, OHTAG) in said algal culture after feeding said algal culture a quantity of 1-dodecanol.
   57. The method of embodiment 49 and further comprising the step of downregulating the expression of diacylglycerol acyl transferase (DGAT2) in said transformed algal cell.
   58. The method of embodiment 57 wherein said step of downregulating the expression of diacylglycerol acyl transferase (DGAT2) in said transformed algal cell comprises the step of transforming said algal cell to express a double-stranded RNA (dsRNA) configured to initiate an RNA-interference mechanism directed to expression of diacylglycerol acyl transferase (DGAT2).
   59. The method of embodiment 49 and further comprising the step of downregulating the expression of very long chain fatty acid (VLCFA) elongases in said transformed algal cell.
   60. The method of embodiment 59 wherein said step of downregulating the expression of at least one very long chain fatty acid (VLCFA) elongase in said transformed algal cell comprises the step of transforming said algal cell to express a double-stranded RNA (dsRNA) configured to initiate an RNA-interference mechanism directed to expression of at least one long chain fatty acid (VLCFA) elongase.
   61. The method of embodiment 49 and further comprising the step of downregulating the expression of fatty acid elongase (FAE).
   62. The method of embodiment 61 wherein said step of downregulating the expression of fatty acid elongase (FAE) comprises the step of transforming said algal cell to express a double-stranded RNA (dsRNA) configured to initiate an RNA-interference mechanism directed to expression of fatty acid elongase (FAE).
   63. The method of embodiment 49 and further comprising the step of increasing expression of pyruvate dehydrogenase in said transformed algal cell to increase production of acetyl-CoA.
   64. The method of embodiment 63 wherein said step of increasing expression of pyruvate dehydrogenase in said transformed algal cell to increase production of acetyl-CoA comprises the step of transforming said algal cell to express a heterologous pyruvate dehydrogenase complex.
   65. The method of embodiment 64 wherein said step of transforming said algal cell to express a heterologous pyruvate dehydrogenase complex comprises the step of transforming said algal cell to express a heterologous pyruvate dehydrogenase complex selected from the group of amino acid sequences SEQ ID NOs. 38-43.
   66. The method of embodiment 49 and further comprising the step of transforming said algal cell to express a heterologous cyanobacterial fructose-1,6-/sedoheptulose-1,7-bisphosphatase (fII).
   67. The method of embodiment 49 wherein said step of transforming said algal cell to express a heterologous cyanobacterial fructose-1,6-/sedoheptulose-1,7-bisphosphatase (fII) comprises the step of transforming said algal cell to express a heterologous cyanobacterial fructose-1,6-/sedoheptulose-1,7-bisphosphatase (fII) according to amino acid sequence SEQ ID NO. 24.
   68. The method of embodiment 49 and further comprising the step of culturing the transformed algal cell under low nitrogen conditions.
   69. A method of novel wax biosynthesis in algae comprising the steps of:
transforming an algal cell with one or more polynucleotide sequences operably linked to a promoter that expresses:
   a heterologous fatty acyl-CoA reductase from Euglena gracilis according to amino acid sequence SEQ ID NO. 5, that reduces long-chain-fatty-acyl-CoA to acyl alcohol or a heterologous fatty acyl-CoA reductase from Simmondsia chinensis according to amino acid sequence SEQ ID NO. 1, that reduces long-chain-fatty-acyl-CoA to acyl alcohol and
   a heterologous acyl-CoA:diacylglycerol acyltransferase from Euglena gracilis according to the amino acid sequence selected from the group consisting of: SEQ ID NO. 9, SEQ ID NO. 11, and 13, that biosynthesizes wax esters from said acyl alcohol;
culturing said algal cell; and
harvesting the biosynthesized wax esters from the algal cell culture.
   70. A recombinant algal cell configured to biosynthesize wax having a polynucleotide sequence operably linked to a promoter wherein said polynucleotide comprises a heterologous nucleotide sequence selected from the group consisting of: SEQ ID NOs. 3-4, 7-8, 10, 12, 14, 25, and 31-34.
   71. A recombinant algal cell configured to biosynthesize wax having a polynucleotide sequence operably linked to a promoter wherein said polynucleotide comprises a heterologous nucleotide sequence encoding a heterologous peptide selected from the group consisting of: SEQ ID NOs. 1-2, 5-6, 9, 13, 14-24, 26-30, and 35-44.
   72. A recombinant algal cell configured to biosynthesize wax having a polynucleotide sequence operably linked to a promoter that expresses a heterologous fatty acyl-CoA reductase (FAR), and a heterologous wax synthase (WS) wherein said FAR and WS operate to biosynthesize wax esters.
   73. A recombinant algal cell configured to biosynthesize wax having a polynucleotide sequence operably linked to a promoter that expresses one or more of the following:
a heterologous fatty acyl-CoA reductase (FAR) from Simmondsia chinensis;
a heterologous fatty acyl-CoA reductase (FAR) from Euglena gracilis;
a heterologous wax synthase (WS) from Simmondsia chinensis; and
a heterologous wax synthase (WS) from Euglena gracilis.
   74. A recombinant algal cell configured to biosynthesize wax having a polynucleotide sequence operably linked to a promoter that expresses one or more of the following:
a heterologous fatty acyl-CoA reductase (FAR) from Simmondsia chinensis;
a heterologous fatty acyl-CoA reductase (FAR) from Euglena gracilis;
a heterologous wax synthase (WS) from Simmondsia chinensis;
a heterologous wax synthase (WS) from Euglena gracilis; and
a heterologous acyl-CoA:diacylglycerol acyltransferase from Euglena gracilis.
   75. A recombinant algal cell configured to biosynthesize wax having a polynucleotide sequence operably linked to a promoter that expresses one or more of the following:
a heterologous fatty acyl-CoA reductase (FAR) according to amino acid sequence SEQ ID NO. 1;
a heterologous fatty acyl-CoA reductase (FAR) according to amino acid sequence SEQ ID NO. 5;
a heterologous wax synthase (WS) according to amino acid sequence SEQ ID NO. 2;
a heterologous wax synthase (WS) according to amino acid sequence SEQ ID NO. 6; and
a heterologous acyl-CoA:diacylglycerol acyltransferase from Euglena gracilis according to the amino acid sequence selected from the group consisting of: SEQ ID NO. 9, SEQ ID NO. 11, and 13.
   76. A recombinant algal cell configured to biosynthesize wax having a polynucleotide sequence operably linked to a promoter that expresses a heterologous fatty acyl-CoA reductase (FAR) is selected from the group consisting of:
a heterologous fatty acyl-CoA reductase (FAR) according to amino acid sequence SEQ ID NO. 1; and
a heterologous fatty acyl-CoA reductase (FAR) according to amino acid sequence SEQ ID NO. 5.
   77. A recombinant algal cell configured to biosynthesize wax having a polynucleotide sequence operably linked to a promoter that expresses a heterologous wax synthase (WS) is selected from the group consisting of:
a heterologous wax synthase (WS) from Simmondsia chinensis; and
a heterologous wax synthase (WS) from Euglena gracilis.
   78. A recombinant algal cell configured to biosynthesize wax having a polynucleotide sequence operably linked to a promoter that expresses a heterologous wax synthase (WS) is selected from the group consisting of:
a heterologous wax synthase (WS) according to amino acid sequence SEQ ID NO. 2; and
a heterologous wax synthase (WS) according to amino acid sequence SEQ ID NO. 6.
   79. A recombinant algal cell configured to biosynthesize wax having a polynucleotide sequence operably linked to a promoter that expresses a heterologous fatty acyl-CoA reductase (FAR) selected from the group consisting of:
a heterologous fatty acyl-CoA reductase (FAR) from Simmondsia chinensis; and
a heterologous fatty acyl-CoA reductase (FAR) from Euglena gracilis.
   80. A recombinant algal cell configured to biosynthesize wax having a polynucleotide sequence operably linked to a promoter that expresses a heterologous fatty acyl-CoA reductase (FAR) is selected from the group consisting of:
a heterologous fatty acyl-CoA reductase (FAR) according to amino acid sequence SEQ ID NO. 1; and
a heterologous fatty acyl-CoA reductase (FAR) according to amino acid sequence SEQ ID NO. 5.
   81. A recombinant algal cell configured to biosynthesize wax having a polynucleotide sequence operably linked to a promoter that expresses a heterologous wax synthase (WS) is selected from the group consisting of:
a heterologous wax synthase (WS) from Simmondsia chinensis; and
a heterologous wax synthase (WS) from Euglena gracilis.
   82. A recombinant algal cell configured to biosynthesize wax having a polynucleotide sequence operably linked to a promoter that expresses a heterologous wax synthase (WS) is selected from the group consisting of:
a heterologous wax synthase (WS) according to amino acid sequence SEQ ID NO. 2;
a heterologous wax synthase (WS) according to amino acid sequence SEQ ID NO. 6;
   83. A recombinant algal cell configured to biosynthesize wax having a polynucleotide sequence operably linked to a promoter that expresses a heterologous fatty acyl-CoA reductase from Euglena gracilis according to amino acid sequence SEQ ID NO. 5, that reduces long-chain-fatty-acyl-CoA to acyl alcohol or a heterologous fatty acyl-CoA reductase from Simmondsia chinensis according to amino acid sequence SEQ ID NO. 1, that reduces long-chain-fatty-acyl-CoA to acyl alcohol and a heterologous acyl-CoA:diacylglycerol acyltransferase from Euglena gracilis according to the amino acid sequence selected from the group consisting of: SEQ ID NO. 9, SEQ ID NO. 11, and 13, that biosynthesizes wax esters from said acyl alcohol.

Additional aims of the invention will become apparent from the specification, claims and figures below.

### BRIEF DESCRIPTION OF DRAWINGS

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. (A) Molecular structures of exemplary petroleum-based waxes (paraffin) and select natural waxes such as bees wax. (B) Wax biosynthesis in jojoba. Jojoba waxes are composed of very long-chain (C20, C22, and C24) monounsaturated fatty acids and alcohols.

FIG. 2. Gene cassette of jojoba fatty acyl-CoA reductase (A) and wax synthase (B) in pChlamy_4 expression vector, driven by the Hsp 70A-Rbc S2 promoter, a strong hybrid constitutive promoter consisting of Hsp70 and RbcS2 promoters. The Sh ble gene product from Streptoalloteichus hindustanus confers resistance to zeocin The 2A peptide from the Foot-and-mouth disease virus (F2A), which mediates a self-cleavage reaction, links transgene expression to zeocin resistance. The DNA and protein sequences of FAR and WS1 are shown in Tables 1 and 2.

FIG. 3. Gene cassette of Euglena gracilis fatty acyl-CoA reductase (A) and wax synthase (B) in pChlamy_4 expression vector, driven by the Hsp 70A-Rbc S2 promoter, a strong hybrid constitutive promoter consisting of Hsp70 and RbcS2 promoters. The synthase (B) can be a wax synthase (WS1) or a dual enzyme with wax synthase and acyl-CoA:diacylglycerol acyltransferase (DGAT) activities. The Sh ble gene product confers resistance to zeocin. The 2A peptide from the Foot-and-mouth disease virus (F2A), which mediates a self-cleavage reaction, links transgene multiple independent expression to zeocin resistance. The DNA and protein sequences of Euglena gracilis fatty acyl-CoA reductase and wax synthase are provided.

FIG. 4. MS/MS fragmentation profiles. Fragmentation structures for (A) WE ISTD C34:0 (17:0/17:0) and (B) WE C42:1 (20:1/22:0). Precursor and product ions for (A) were 526.5556 and 271.2892, respectively, and for WE C42:1 (B) were 636.5536 and 341.3076, respectively.

FIG. 5. Wax production in WT C. reinhardtii and transgenic lines overexpressing jojoba FAR and WS (JJFW1, JJFW3, JJFW4, JJFW5 and JJFW10). The concentration of wax ester is calculated in μg wax ester/mg dried biomass, where ng wax ester is approximated from to the standard used, WE C34:0 (17:0/17:0).

FIG. 6. Effect of serial nitrogen deprivation on the yield of was esters in transgenic C. reinhardtii (JJFW5). Average values and standard error bars are shown. The concentration of wax ester is calculated in μg wax ester/mg dried biomass, where ng wax ester is approximated from the standard used, WE C34:0 (17:0/17:0).

FIG. 7. Production of C34:2 wax ester species in transgenic lines fed with 25 and 50 uM the fatty alcohol 1-dodecanol (C₁₂H₂₆O). The concentration of wax ester is calculated in ng wax ester/mg dried biomass, where μg wax ester is approximated from the standard used, WE C34:0 (17:0/17:0).

FIG. 8. Effect of 1-dodecanol feeding (50 μM) on production of (A) WE C34:2 and (B) WE C42:1 in EgFWC1, EgFWC2 and EgWS-TZ3 transgenic lines vs. unfed cultures. The concentration of wax ester is calculated in ng wax ester/mg dried biomass, where μg wax ester is approximated from the standard used, WE C34:0 (17:0/17:0).

FIG. 9. Fragmentation structures of (A) WE ISTD C34:0 (17:0/17:0) and (B) WE C42:1 (20:1/22:0). Precursor and product ions observed for the ISTD (A) are 526.5602 and 271.2511, respectively, and for (B) are 636.6697 and 341.3294 m/z, respectively.

FIG. 10. Proposed route for ethanol to fuels and feedstock molecules.

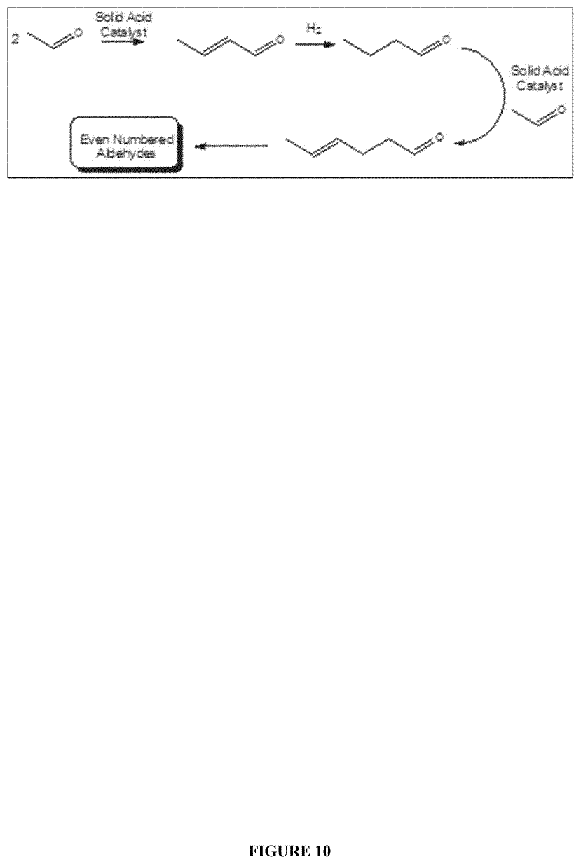

FIG. 11. Gas chromatogram following self aldol chain extension (Pd/C, solid acid, 100° C., 50 psi H₂, 120 mins).

FIG. 12. Exemplary biosynthesis of major wax components.

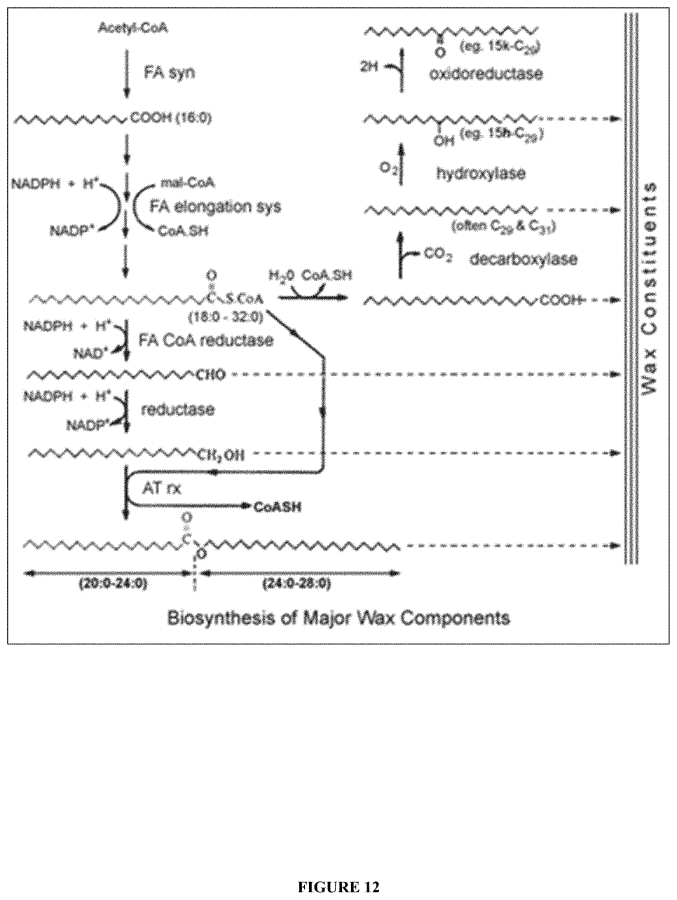

FIG. 13. Wax synthesis in Euglena under anaerobic conditions and wax metabolism under aerobic conditions.

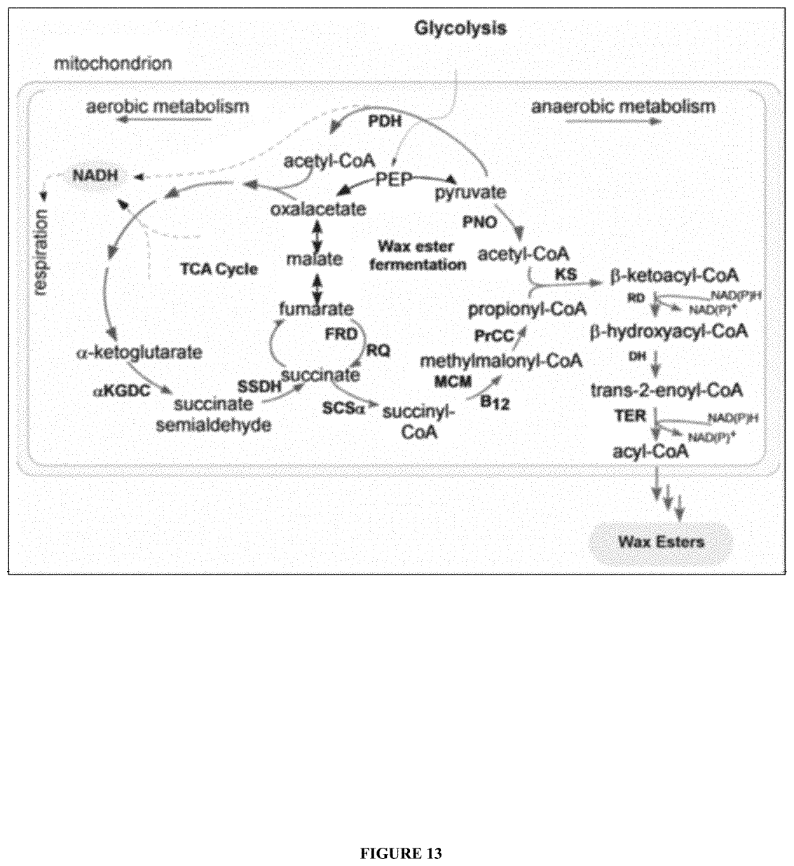

FIG. 14. Schematic of very-long-chain fatty acid elongation.

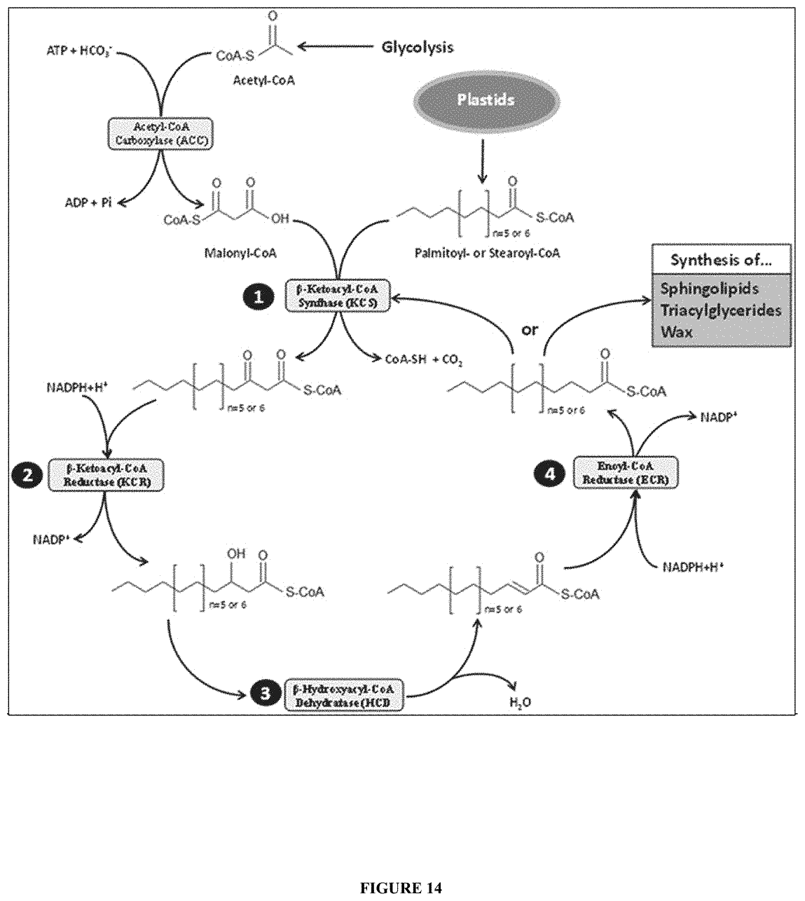

FIG. 15. Gene expression analysis of fatty acyl-CoA reductase (FAR) and wax synthase (WS1) from the desert shrub jojoba. Arrows indicated the expected bands compared to the 1 kb Plus ladder. Lane numbers correspond to the lines JJFW1, 2, 3, 4, 5 and 10).

FIG. 16. Gene expression analysis of fatty acyl-CoA reductase (FAR, 1) and wax synthase (WS, 2) from Euglena gracilis in Chlamydomonas reinhardtii. Numbers indicate FAR expression (1) and WS expression (2).

FIG. 17. Effect of serial nitrogen deprivation (0, 25, 50, 75, 100% N) on the lipid profile in transgenic C. reinhardtii (JJFW5). Corrected ion intensity values were used to examine alterations between treatments for diacylglycerol (DAG), digalactosyl diacylglycerol, epoxy triacylglycerol (ETAG), hydroxylated triacylglycerol (OHTAG), monogalactosyl diacylglycerol (MGDG), phophatidylcholine (PC), phosphatidylethanolamine (PE), phosphatidylglycerol (PG), phosphatidylinositol (PI), and triacylglycerol (TAG).

FIG. 18. Effect of dodecanol feeding (50 μM) on lipid profiles on WT and transgenic lines JJFW4 and JJFW5. Corrected ion intensity values were used to examine alterations between treatments for (A) phophatidylcholine (PC) and phosphatidylglycerol (PG), and for (B) diacylglycerol (DAG), digalactosyl diacylglycerol, epoxy triacylglycerol (ETAG), hydroxylated triacylglycerol (OHTAG), monogalactosyl diacylglycerol (MGDG), phosphatidylethanolamine (PE), phosphatidylinositol (PI), and triacylglycerol (TAG).

FIG. 19. WE C42:1 content (μg/mg) in large volume cultures of JJFW5. The concentration of wax ester is calculated in μg wax ester/mg dried biomass, where μg wax ester is approximated from the standard used, WE C34:0 (17:0/17:0).

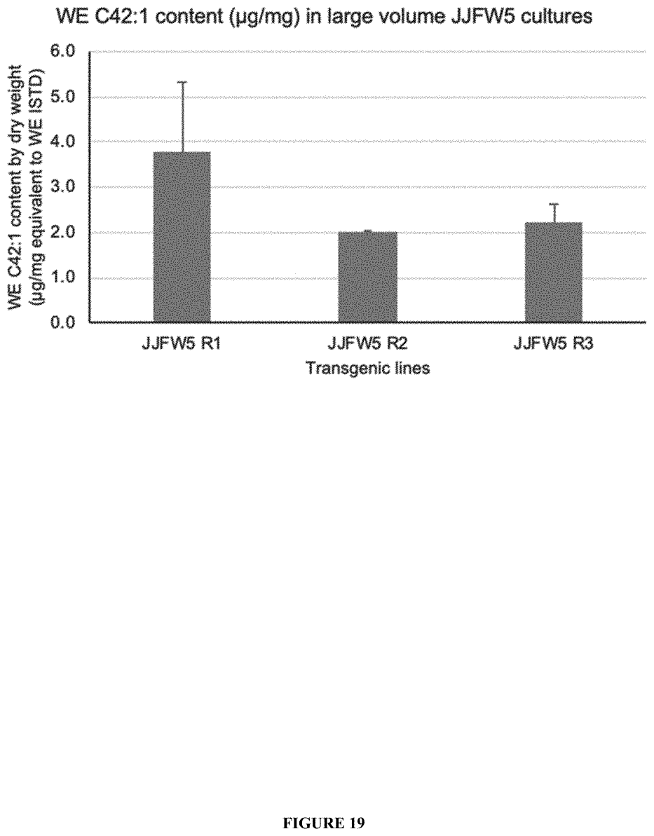

### MODE(S) FOR CARRYING OUT THE INVENTION(S)

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries.

Accordingly, in one aspect, the inventive technology provides a method for modulating the production of molecules of interest in a micro-organism, in particular a microalga, said method comprising culturing a recombinant micro-organism, in particular a recombinant microalga, which has been genetically engineered to produce or overproduce waxes in said genetically modified micro-organism. In particular embodiments, the invention relates to a method for the production of molecules of interest, which encompasses the steps of: (i) genetically engineering a micro-organism, in particular a microalga, to produce or overproduce waxes; and (ii) culturing the recombinant micro-organism, in particular the recombinant microalga, obtained in step (i) so as to allow the production of said molecules of interest.

In particular embodiments, the molecules of interest are molecules of the wax biosynthesis pathway or biomolecules derived from said molecules and the production of such molecules of interest is increased according to the invention. In particular embodiments, the recombinant micro-organism has been engineered to express or overexpress a protein involved in a wax biosynthesis pathway. Preferably, the recombinant micro-organism has been transformed with a recombinant nucleic acid encoding a protein involved in a wax biosynthesis pathway.

Additional embodiments may include the reduction in the expression of certain molecules of interest. In some embodiments, suppression of these molecules may divert fatty acids to wax production. Preferably, the recombinant micro-organism has been transformed with a recombinant nucleic acid encoding a dsRNA targeted to downregulate expression of one or more genes in the fatty-acid biosynthesis pathway. Accordingly, in embodiments, the method encompasses transforming the micro-organism with a recombinant nucleic acid encoding a protein involved in a wax biosynthesis pathway, and culturing the recombinant micro-organism under conditions suitable to produce or overproduce select waxes in said recombinant micro-organism so as to allow production of the desired molecule or biomolecule by the micro-organism.

More specifically, disclosed herein are methods and compositions for the enhanced production of waxes in algae. Methods for identifying one or more gene(s) involved in the biosynthesis of waxes in algae, as well methods and compositions for the modulation of their expression are also provided. Methods for identifying one or more gene(s) for use as a target gene for enhanced siRNA-mediated interference are also provided. DNA constructs encoding inhibitory RNA molecules may be designed to suppress one or more target gene(s) that may result in enhanced wax production and accumulation in algae. Genetically modified algal strains that may be engineered to efficiently modulate expression of select genes resulting in enhanced production of waxes, as well as deliver inhibitory RNA molecules are also described in the present invention.

In particular embodiments, one or more enzymes that control wax biosynthesis may be been up-regulated or down-regulated to improve wax production. Up-regulation can be achieved, for example, by transforming cells with an expression vector in which a gene encoding the enzyme of interest is expressed, e.g., using a strong inducible promoter and/or enhancer elements that increase transcription. Such constructs can include a selectable marker such that the transformants can be subjected to selection, which can result in amplification of the construct and an increase in the expression level of the encoded enzyme.

Examples of enzymes suitable for up-regulation according to the methods of the invention include fatty acyl-CoA reductase (FAR) which is involved in the reduction of very long chain fatty acids-CoA (VLCFA-CoA) molecules to acyl alcohols. Up-regulation of very long chain fatty acids-CoA can increase production of acyl alcohols, and thereby increase wax biosynthesis. Fatty acid production can also be increased by up-regulation of wax synthases (WS) that are involved in the biosynthesis of wax esters from the acyl alcohols. Up-regulation of this class of enzymes can increase wax biosynthesis.

In yet another embodiment the levels of acetyl-CoA production for enhanced fatty acid production may be enhanced by elevating pyruvate dehydrogenase levels.

During wax biosynthesis, very long chain fatty acid VLCFA-CoA molecules may be subsequently reduced to acyl alcohols by a fatty acyl-CoA reductase (FAR). The acyl alcohols are then used to synthesize wax esters by wax synthases (WS). The introduction and overexpression of these enzymes in microalgae may result in increased wax biosynthesis and accumulation.

In one embodiment, the invention may include the generation of a transgenic strain of microalgae that may overexpress one or more fatty acyl-CoA reductase (FAR) and/or wax synthase (WS) genes. In this embodiment, one or more homologous and/or heterologous fatty acyl-CoA reductase (FAR) and wax synthase (WS) genes may be introduced into a microalgae. In one preferred embodiment, heterologous fatty acyl-CoA reductase (FAR) and/or wax synthase (WS) genes may be used to generate transgenic microalgae. In this preferred embodiment, fatty acyl-CoA reductase (FAR) and/or wax synthase (WS) genes from one or more organisms may be heterologously expressed into a microalgae. Exemplary organisms may be selected from the group consisting of: Jojoba, sorghum, Arabidopsis, palm tree (Copernicia prunifera), and Euglena may be expressed in a fast growing microalgae, such as Chlamydomonas reinhardtii or Chlorella. In alternative embodiments, heterologous fatty acyl-CoA reductase (FAR) and/or wax synthase (WS) genes from one or more strains of algae may be introduced into a different microalgae, such as Chlamydomonas reinhardtii or Chlorella among others.

Accordingly, in embodiments of the methods described herein, a recombinant microalga may be transformed with a recombinant nucleic acid encoding a fatty acyl-CoA reductase (FAR) protein. In particular embodiments, the recombinant nucleic acid encode a fatty acyl-CoA reductase (FAR) protein from Jojoba (Simmondsia chinensis) or a variant or a homolog thereof. In this embodiment, the recombinant micro-organism may be transformed with a recombinant nucleic acid according to SEQ ID NO. 2 which may encode a protein having the sequence of SEQ ID NO. 1, or a sequence substantially identical to SEQ ID NO. 1, or a sequence having at least about 70%, preferably at least about 80%, more preferably at least about 85%, 90% or 95%, even more preferably at least about 96%, 97%, 98% or 99% sequence identity to SEQ ID NO. 1.

Accordingly, in embodiments of the methods described herein, a recombinant microalga may be transformed with a recombinant nucleic acid encoding a fatty acyl-CoA reductase (FAR) protein. In particular embodiments, the recombinant nucleic acid encode a fatty acyl-CoA reductase (FAR) protein from Euglena gracilis or a variant or a homolog thereof. In this embodiment, the recombinant micro-organism may be transformed with a recombinant nucleic acid according to SEQ ID NO. 7 which may encode a protein having the sequence of SEQ ID NO. 5, or a sequence substantially identical to SEQ ID NO. 5, or a sequence having at least about 70%, preferably at least about 80%, more preferably at least about 85%, 90% or 95%, even more preferably at least about 96%, 97%, 98% or 99% sequence identity to SEQ ID NO. 5.

Accordingly, in embodiments of the methods described herein, a recombinant microalga may be transformed with a recombinant nucleic acid encoding a fatty acyl-CoA reductase (FAR) protein. In particular embodiments, the recombinant nucleic acid encode a fatty acyl-CoA reductase (FAR) protein from Arabidopsis or a variant or a homolog thereof. In this embodiment, the recombinant micro-organism is transformed with a recombinant nucleic acid comprising coding for a protein having the sequence of SEQ ID NO. 15, or a sequence substantially identical to SEQ ID NO. 15, or a sequence having at least about 70%, preferably at least about 80%, more preferably at least about 85%, 90% or 95%, even more preferably at least about 96%, 97%, 98% or 99% sequence identity to SEQ ID NO. 15. It should be noted that SEQ ID NO. 15, which encodes fatty acid reductase 1 (FAR1) (Arabidopsis thaliana) is exemplary only. For example in alternative embodiments, FAR genes and their variants and homologs from Arabidopsis thaliana may include but not be limited those exemplary fatty acid reductase 1 genes, and their homologs, identified in Table 2 below. Again, such non-limiting heterologous genes are merely exemplary in nature as a variety of fatty acyl-CoA reductase (FAR) (or fatty acid reductase, the terms being generally interchangeable) are included within the scope of the inventive technology. Examples may include FAR genes/proteins, as well as their variants and homologs from a variety of sources, such as sorghum, Arabidopsis, and palm tree. Additional embodiments may include heterologous and/or homologous FAR genes as generally described herein.

Accordingly, in embodiments of the methods described herein, a recombinant microalga may be transformed with a recombinant nucleic acid encoding a wax synthase (WS) protein. In particular embodiments, the recombinant nucleic acid encode a wax synthase (WS) protein from Jojoba (Simmondsia chinensis) or a variant or a homolog thereof. In this embodiment, the recombinant micro-organism is transformed with a recombinant nucleic acid according to SEQ ID NO. 4, which may encode a protein having the sequence of SEQ ID NO. 2, or a sequence substantially identical to SEQ ID NO. 2, or a sequence having at least about 70%, preferably at least about 80%, more preferably at least about 85%, 90% or 95%, even more preferably at least about 96%, 97%, 98% or 99% sequence identity to SEQ ID NO. 2.

In further embodiments of the methods described herein, a recombinant microalga may be transformed with a recombinant nucleic acid encoding a wax synthase (WS) protein. In particular embodiments, the recombinant nucleic acid encode a wax synthase (WS) protein from E. gracilis or a variant or a homolog thereof. In this embodiment, the recombinant micro-organism is transformed with a recombinant nucleic acid according to SEQ ID NO. 8, which may encode a protein having the sequence of SEQ ID NO. 6, or a sequence substantially identical to SEQ ID NO. 6, or a sequence having at least about 70%, preferably at least about 80%, more preferably at least about 85%, 90% or 95%, even more preferably at least about 96%, 97%, 98% or 99% sequence identity to SEQ ID NO. 6.

In still further embodiments of the methods described herein, a recombinant microalga may be transformed with a recombinant nucleic acid encoding a wax synthase (WS) protein. In particular embodiments, the recombinant nucleic acid encode a wax synthase (WS) protein from Arabidopsis thaliana or a variant or a homolog thereof. In this embodiment, the recombinant micro-organism is transformed with a recombinant nucleic acid which may encode a protein having the sequence of SEQ ID NO. 16, or a sequence substantially identical to SEQ ID NO. 16, or a sequence having at least about 70%, preferably at least about 80%, more preferably at least about 85%, 90% or 95%, even more preferably at least about 96%, 97%, 98% or 99% sequence identity to SEQ ID NO. 16.

It should be noted that SEQ ID NO. 16, which encodes the wax synthase O-acyltransferase WSD1 (Arabidopsis thaliana) is exemplary only. For example in alternative embodiments, WSD genes and their variants and homologs from Arabidopsis thaliana may include but not be limited to exemplary wax synthase genes, and their homologs, identified in Table 3 below. Again, such non-limiting heterologous genes are merely exemplary in nature as a variety of wax synthases (WS) may be included within the scope of the inventive technology. Examples may include WS genes/proteins, as well as their variants and homologs from a variety of sources, such as sorghum, Arabidopsis, and palm tree. Additional embodiments may include heterologous and/or homologous WS genes from algae.

In one preferred embodiment, fatty acyl-CoA reductase (FAR) and wax synthase (WS) genes both be heterologously expressed in a microalga. In this embodiment, the fatty acid reductase (FAR) and/or wax synthase (WS) genes may be part of an artificial genetic construct or expression vector and may further be operably linked to one or more promoters. In this alternative preferred embodiment, fatty acyl-CoA reductase fatty acid reductase (FAR) and/or wax synthase (WS) genes from algae may be expressed into a separate fast growing strain of microalgae, such as Chlamydomonas reinhardtii. Accordingly, in embodiments of the methods described herein, a recombinant microalga may be transformed with a recombinant nucleic acid encoding fatty acyl-CoA reductase (FAR), according to SEQ ID NOs. 3 or 7, and wax synthase (WS) according to SEQ ID NOs. 4 or 8. In particular embodiments, the recombinant nucleic acid encoding fatty acyl-CoA reductase (FAR) and wax synthase (WS) proteins from Jojoba or E. gracilis, among others, or variants or homologs thereof.

In this embodiment, the recombinant micro-organism is transformed with a recombinant nucleic acid coding for a fatty acyl-CoA reductase (FAR) protein having the sequence of SEQ ID NOs. 1 or 5, or an amino acid sequence substantially identical to SEQ ID NOs 1 or 5, or a sequence having at least about 70%, preferably at least about 80%, more preferably at least about 85%, 90% or 95%, even more preferably at least about 96%, 97%, 98% or 99% sequence identity to SEQ ID NOs 1 or 5 respectively. The recombinant micro-organism described above may further be transformed with a recombinant nucleic acid coding for a wax synthase (WS) protein having the sequence of SEQ ID NOs. 2 or 6, or an amino acid sequence substantially identical to SEQ ID NOs 2 or 6, or a sequence having at least about 70%, preferably at least about 80%, more preferably at least about 85%, 90% or 95%, even more preferably at least about 96%, 97%, 98% or 99% sequence identity to SEQ ID NOs 2 or 6 respectively.

In one preferred embodiment, a pyruvate dehydrogenase (PDH) gene may be heterologously expressed in a microalga. Over a sequence of reactions, PDH irreversibly converts pyruvate and NAD⁺ into acetyl-CoA, NADH, and carbon dioxide. The acetyl-CoA enters the citric acid cycle. Acetyl-CoA may also be used to drive multiple anabolic processes, including the production of waxes. Pyruvate dehydrogenase comprises 2 subunits making a trimer. Notably, Pyruvate dehydrogenase is hyper-conserved with, for example <2% divergence in mammalian homologs.

In this embodiment, the pyruvate dehydrogenase (PDH)) gene may be part of an artificial genetic construct or expression vector and may further be operably linked to one or more promoters. In this alternative preferred embodiment, a pyruvate dehydrogenase (PDH) gene from algae may be expressed into a separate fast growing strain of microalgae, such as Chlamydomonas reinhardtii. Accordingly, in embodiments of the methods described herein, a recombinant microalga may be transformed with a recombinant nucleic acid encoding pyruvate dehydrogenase (PDH), according to SEQ ID NOs. 38-43. In particular embodiments, the recombinant nucleic acid encoding PDH protein from SEQ ID NOs. 38-43, among others, or variants or homologs thereof.

In this embodiment, the recombinant micro-organism is transformed with a recombinant nucleic acid coding for a pyruvate dehydrogenase (PDH) protein having the sequence of SEQ ID NOs. 38-43 or an amino acid sequence substantially identical to SEQ ID NOs. 38-43 or a sequence having at least about 70%, preferably at least about 80%, more preferably at least about 85%, 90% or 95%, even more preferably at least about 96%, 97%, 98% or 99% sequence identity to SEQ ID NOs. 38-43, respectively.

Notably, in preferred embodiments, nucleic acid sequences may be codon optimized to be expressed in select algal strains, such as Chlamydomonas.

In embodiments of the invention described herein, a recombinant microalga may be transformed with a recombinant nucleic acid encoding a fatty acid reductase (FAR) and/or wax synthase (WS) genes which may further be part of a genetic construct or expression vector that is operably linked to one or more promoters. In one preferred embodiment, the expression of one or more FAR and/or WS proteins may be operably linked to an inducible promoter. For example, in a preferred embodiment, a microalga may be transformed with an expression vector encoding one or more a fatty acid reductase (FAR) and/or wax synthase (WS) genes operably linked to an inducible promoter. This inducible promoter may include nitrate reductase (NR) or micronutrient (Ni and Fe) inducible gene promoters. Examples of such inducible promoters may include: wild-type or modified nitrate reductase, CYC6, Cpx, CRR1 promoter (for Ni) and/or Fea1 (for iron). In one embodiment, an expression cassette may be operably linked to a NIT1 promoter (SEQ ID NO. 22), or a copper-inducible CYC6 promoter (SEQ ID NO. 23) from Chlamydomonas reinhardtii.

The present invention also generally relates to inhibition of molecules of interest, in particular the inhibition of molecules of the lipid metabolic pathway, including production of triacylglycerol (TAG) and any intermediates in the lipid metabolic pathway, in microorganisms, in particular in microalgae. As used herein, “triacylglycerols”, also referred to as “triacylglycerides” or “TAGs” are esters resulting from the esterification of the three hydroxyl groups of glycerol, with three fatty acids. Microalgae have the ability to accumulate significant amounts of lipids, primarily in the form of triacylglycerol (TAG), especially under stress conditions like nutrient limitation, temperature, pH, or light stress. This accumulation of lipids, in particular TAG, which are used as carbon and energy provisions.

According to one aspect, the present invention provides a method of down-regulating a TAG synthesis gene(s) by sequence homology targeting in a microalga cell and a nucleic acid construct for use in this method, as well as an inhibitory RNA polynucleotide, such as a hpRNA or annealed dsRNA, for use in the nucleic acid construct. The method comprises introducing into the algal cell a nucleic acid construct capable of producing inhibitory RNA and expressing the nucleic acid construct for a time sufficient to produce siRNAs (small interfering RNAs) or microRNA (miRNA), wherein the siRNA/miRNA inhibits expression of the target TAG synthesis gene or sequence. Here, miRNA constructs comprise a polynucleotide encoding a modified RNA precursor capable of forming a double-stranded RNA (dsRNA) or a hairpin (hpRNA), wherein the modified RNA precursor comprises a modified miRNA and a sequence complementary to the modified miRNA, wherein the modified miRNA is a miRNA modified to be (i) fully or partially complementary to the target sequence. As is well known in the art, the pre-miRNA forms a hairpin which in some cases the double-stranded region may be very short, e.g., not exceeding 21-25 bp in length. The nucleic acid construct may further comprise a promoter operably linked to the polynucleotide.

As used herein, interfering RNA or RNA interference (RNAi) is a biological mechanism which leads to post transcriptional gene silencing (PTGS) triggered by double-stranded RNA (dsRNA) molecules, for example provided by hpRNA, to prevent the expression of specific genes. For example, in one preferred embodiment, RNA interference may be accomplished as short hpRNA molecules may be imported directly into the cytoplasm, anneal together to form a dsRNA, and then cleaved to short fragments by the Dicer enzyme. This enzyme Dicer may process the dsRNA into ˜21-22-nucleotide fragment with a 2-nucleotide overhang at the 3′ end, small interfering RNAs (siRNAs). The antisense strand of siRNA become specific to endonuclease-protein complex, RNA-induced silencing complex (RISC), which then targets the homologous RNA and degrades it at specific site that results in the knock-down of protein expression.

In embodiments of the invention described herein, a recombinant microalga may be transformed with a recombinant nucleic acid encoding an interfering RNA molecule that may be configured to inhibit or suppress synthesis of triacylglycerol (TAG). More particularly, the inventive technology provides methods for RNA-based inhibition of TAG production in microorganisms, in particular the microalgae. In various embodiments, siRNAs may be configured to target nucleotide sequences for diacylglycerol acyl transferase (DGAT) gene and/or family members including variant and homologs in algae resulting in the disruption of TAG synthesis. In this embodiment, fatty acids in the genetically modified microalgae, instead of being used to generate TAG's, may be directed to the wax biosynthetic pathways increasing select wax production and accumulation.

In one embodiment, diacylglycerol O-acyltransferase homolog 2 (DGAT2) and/or variants or homologs of the same may be targeted for RNA mediated inhibition. In this preferred embodiment an expression vector encoding one or more hairpin RNA/dsRNA molecules targeting the DGAT2 family coding RNAs for degradation may be expressed in transgenic microalgae. Expression of these inhibitory RNA molecules may result in the reduction of the encoded protein accumulation levels for the DGAT2 family of genes. This may be accomplished through transformation of microalgae with an expression vector carrying a nucleotide construct encoding the regulatory dsRNA homologous to one or more DGAT2 coding or regulatory RNA sequences. In one example, an expression vector carrying a nucleotide construct encoding the regulatory dsRNA homologous to one or more DGAT2 coding or regulatory RNA sequences of SEQ ID NO. 17 may be introduced to a microalga cell precipitating an RNA-based interference cascade regulated by an inducible gene promoter and ultimately resulting in TAG synthesis disruption. This reduction or inhibition of TAG formation may allow greater then wild-type shunting of fatty acids to move toward wax biosynthesis pathways and increase the cells overall wax production and accumulation capacity. It should be noted that SEQ ID NO. 17 is an exemplary DGAT2 protein sequence only, and not meant to be liming in any way. Specifically contemplated in the invention are a number of DGAT, DGAT2 genes as well as their variant and homologs. In particular, diacylglycerol acyl transferases such as DGAT and DGAT2, and their variants and homologs in microalgae and in particular the conserved regions between the target genes in, for example Arabidopsis thaliana genes sequences and the target genes in microalgae such as Chlamydomonas reinhardtii.

Preferably, the expression of the target gene (as measured by the expressed RNA or protein) is reduced, inhibited or attenuated by at least 10%, preferably at least 30% or 40%, preferably at least 50% or 60%, more preferably at least 80%, most preferably at least 90% or 95% or 100%.

In one embodiment, addition of the elongase inhibitor flufenacet to the algal growth medium may specifically reduce the accumulation of odd-numbered fatty acids and alcohols and tended to increase the overall yield of anaerobic wax esters. Addition of the elongase inhibitor flufenacet to the algal growth medium may specifically reduce the accumulation of odd-numbered fatty acids and alcohols and tended to increase the overall yield of anaerobic wax esters.

In another embodiment of the invention, one or more strains of algae may be genetically modified to suppress fatty acid elongase (FAE) activity in algae. Very-long-chain fatty acids (VLCFA), formally defined as fatty acids longer than 18 carbons, are extended by an ER membrane-embedded protein complex of 4 enzymes, acting presumably on the cytosolic side. Fatty acid elongase (FAE) activity results in successive action of β-ketoacyl-CoA synthase (KCS), β-ketoacyl-CoA reductase (KCR), β-hydroxyacyl-CoA dehydratase (HCD), and enoyl-CoA reductase (ECR). To accomplish this elongation activity, each of these FAE associated enzymes utilizes as substrate the product of the previous one in cycles beginning by malonyl-CoA condensation to long-chain acyl-CoA.

As noted above, suppression of VLCFA elongases may result in increased production and accumulation of wax constituents, such as wax esters. As such, in one preferred embodiment, very long chain fatty acid (VLCFA) elongases as generally outlined above may be suppressed in a microorganism, such as a microalgae. In a preferred embodiment, this suppression may be through the production of dsRNA regulated by an inducible gene promoter and configured to target fatty acid elongases (FAE), and/or related gene family members in algae involved in fatty acid, or very long chain fatty acid (VLCFA) production. Examples of such target elongases may include one or more of KCS (SEQ ID NO. 18), KCR (SEQ ID NO. 19), HCD (SEQ ID NO. 20), ECR (SEQ ID NO. 21) (collectively FAE target genes).

Preferably, the expression of the FAE target gene(s) (as measured by the expressed RNA or protein) is reduced, inhibited or attenuated by at least 10%, preferably at least 30% or 40%, preferably at least 50% or 60%, more preferably at least 80%, most preferably at least 90% or 95% or 100%. In certain embodiments, production for such dsRNA targeting FAE genes may be operably linked to one or more promoters as generally described above.

As outlined above, in the production of wax the VLCFA-CoA molecules are reduced to acyl alcohols by a fatty acyl-CoA reductase (FAR). The acyl alcohols are then used to synthesize wax esters by wax synthases (WS). In one embodiment of the invention, non-naturally occurring synthetic and or semi-synthetic acyl alcohols may be generated and fed to wild-type or genetically modified microalgae. In this embodiment, these novel acyl alcohols may be incorporated into wax biosynthetic pathways generating novel waxes with extended chain lengths, branched alkanes to alter packing and melting potential, and amphipathic acyl alcohols to manipulate surface properties (hydrophilicity) and physical properties (melting point and hardness). In additional embodiments, these novel acyl alcohols may be isotopically labeled and fed to wild-type or genetically modified microalgae.

Notably, cellulosic ethanol can be readily converted to acetaldehyde which we will subject to aldol condensation catalysts to generate long chain acyl alcohols as potential feedstocks for wax production. This involves the aldol condensation of acetaldehyde using solid acid catalysts which we have shown will form crotonaldehyde. Hydrogenation of this molecule is facile and the resultant butryladehyde can then undergo additional aldol condensation reactions, growing the chain length and can be considered a controlled polymerization of acetaldehyde. The subsequent aldehydes can then be readily converted to alcohols. The uptake of these synthetic molecules may be tracked by incorporating stable isotopes (i.e. ¹²C or ¹³C) using isotopically labelled ethanol or acetaldehyde as a starting molecule. In this embodiment, such isotopically labelled molecules may allow for the tracking of the uptake and use of the synthetic starting molecule by a cell, and its eventual incorporation into a wax product.

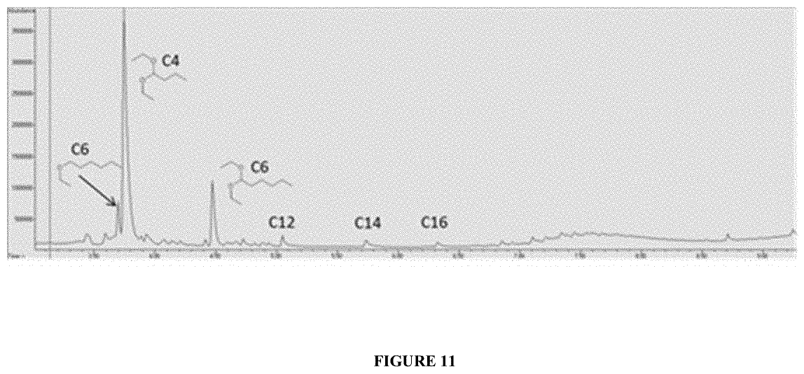

Generally referring to FIG. 11, in certain embodiments, synthetic generation of long-chain pre-cursor molecules may be initiated using chain extension starting with acetaldehyde under very mild conditions using a solid acid catalyst. With ethanol solutions of acetaldehyde, a solid acid catalyst and 50 psi H₂ with Pd/C as the hydrogenation catalyst in a sealed reaction vessel heating at 100° C. for 120 minutes results in complete conversion of acetaldehyde and the formation of molecules with between 4 and 16 carbons as the main products as evidenced in the GC-MS of the crude reaction mixtures. Further heating for a total of 5 hours gives heavier molecules with at least 24 carbon atoms exhibiting linear and branched chains. Branched chain alcohols are typically non-metabolizable so they will be incorporated into the wax rather than consumed by the algae favoring the synthetic production of branched alcohols over linear. As such, using simple catalytic approaches the synthesis of long chain branched alcohols suitable for uptake by microalgae and subsequent production of waxes may be accomplished. In further embodiments, the inclusion of such synthetic and novel acyl alcohols, may allow for the design and tailoring of the properties of waxes via subtle variation of the acyl alcohol inputs. Additional embodiment may allow for the generation of novel acyl alcohol molecules through shorter chain intermediary molecules by alternative aldol or Guerbet reactions.

Certain embodiments of the inventive technology described herein, include the semi-synthesis of novel wax compounds. In this embodiment, semi-synthetic, synthetic and/or novel acyl alcohols, (novel meaning acyl alcohols that are not produced by a WT host cell) could be fed to microalgae and incorporated into wax biosynthetic pathways. The successful incorporation of acyl alcohols into waxes may be modulated based on the range of substrates that the wax synthases (WS) can utilize, whether acyl alcohols are toxic to algae and/or interfere with other metabolic processes, and whether they can compete effectively with natural substrates produced by the algae and to what magnitude.

In a preferred embodiment, the invention may include the synthesis of both naturally used and novel acyl alcohols that may be isotopically labeled with ¹³C. These substrates may be fed to a microalgae culture under conditions previously developed for optimal wax synthesis at a range of concentrations so as to determine the optimal concentration for maximum incorporation into wax. Waxes may then be extracted and characterized by mass spectroscopy for incorporation of ¹³C-labeled natural acyl alcohol substrates into waxes to determine their competitiveness relative to in vivo synthesized acyl alcohols for incorporation into wax. In addition, it may determined by MS whether novel ¹³C-labeled acyl alcohols are incorporated into waxes, at what rate, and what yield relative to natural ¹³C labeled acyl alcohols. These novel waxes could be selected for improved performance properties in Coatings and other applications.

Additional embodiments may include the incorporation of the semi-synthesis of novel wax compounds in genetically modified microalgae. For example, in certain embodiments, fatty acid elongase activity (FAE) family members involved in VLCFA for wax synthesis may be inhibited using dsRNA mediated interference as generally described herein. In this embodiment, semi-synthetic, synthetic and/or novel acyl alcohols, may be fed to such genetically engineered microalgae and incorporated into wax biosynthesis pathways(s) resulting in the production of novel or enhanced wax products.

Additional embodiments may include the incorporation of semi-synthesis of novel wax compounds in genetically modified microalgae. For example, in certain embodiments, separately from, or in addition to the inhibition of fatty acid elongase activity (FAE) family members, one or more heterologous wax synthase (WS) (SEQ ID NOs. 2 or 6) or fatty acyl-CoA reductase (FAR) (SEQ ID NOs. 1 or 5) enzymes may be expressed in a transgenic microalgae strain. The term “algae” “microalga” or “microalgae” (plural) as used herein refers to microscopic algae. “Microalgae” encompass, without limitation, organisms within: (i) several eukaryotic phyla, including the Rhodophyta (red algae), Chlorophyta (green algae), Dinoflagellata, Haptophyta, (ii) several classes from the eukaryotic phylum Heterokontophyta which includes, without limitation, the classes Bacillariophycea (diatoms), Eustigmatophycea, Phaeophyceae (brown algae), Xanthophyceae (yellow-green algae) and Chrysophyceae (golden algae), and (iii) the prokaryotic phylum Cyanobacteria (blue-green algae). The term “microalgae” includes for example selected from: Achnanthes, Amphora, Anabaena, Anikstrodesmis, Arachnoidiscusm, Aster, Botryococcus, Chaetoceros, Chlamydomonas, Chlorella, Chlorococcum, Chorethron, Cocconeis, Coscinodiscus, Crypthecodinium, Cyclotella, Cylindrotheca, Desmodesmus, Dunaliella, Emiliana, Euglena, Fistulifera, Fragilariopsis, Gyrosigma, Hematococcus, Isochrysis, Lampriscus, Monochrysis, Monoraphidium, Nannochloris, Nannochloropsis, Navicula, Neochloris, Nephrochloris, Nephroselmis, Nitzschia, Nodularia, Nostoc, Odontella, Oochromonas, Oocystis, Oscillartoria, Pavlova, Phaeodactylum, Playtmonas, Pleurochrysis, Porhyra, Pseudoanabaena, Pyramimonas, Scenedesmus, Schyzochitrium, Stichococcus, Synechococcus, Synechocystis, Tetraselmis, Thalassiosira, and Trichodesmium, Auxenchlorella protothecoides.

The term wax as used herein includes a variety of fatty acid esters which form solids or pliable substances under an identified set of physical conditions. For example, a wax generally forms a pliable substance at room temperature. The term wax may also be referred to in some embodiments as a “wax ester.”

The term “transformation” means introducing an exogenous nucleic acid into an organism so that the nucleic acid is replicable, either as an extrachromosomal element or by chromosomal integration. The terms “transgenic,” or “genetically engineered,” or “genetically modified,” or “recombinant” as used herein with reference to a host cell, in particular a micro-organism such as a microalga, denote a non-naturally occurring host cell, as well as its recombinant progeny, that has at least one genetic alteration not found in a naturally occurring strain of the referenced species, including wild-type strains of the referenced species. Such genetic modification is typically achieved by technical means (i.e. non-naturally) through human intervention and may include, e.g., the introduction of an exogenous nucleic acid and/or the modification, over-expression, or deletion of an endogenous nucleic acid.

The term “exogenous,” “heterologous” or “foreign” as used herein is intended to mean that the referenced molecule, in particular nucleic acid, is not naturally present in the host cell. The term “endogenous,” “homologous” or “native” as used herein denotes that the referenced molecule, in particular nucleic acid, is present in the host cell.

The term “nucleic acid” or “nucleic acid molecules” include single- and double-stranded forms of DNA; single-stranded forms of RNA; and double-stranded forms of RNA (dsRNA).

The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (microRNA), hpRNA (hairpin RNA), tRNA (transfer RNA), whether charged or discharged with a corresponding acylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

As used herein “hairpin RNA” (hpRNA) refers to any self-annealing double-stranded RNA molecule. In its simplest representation, a hairpin RNA consists of a double stranded stem made up by the annealing RNA strands, connected by a single stranded RNA loop, and is also referred to as a, “pan-handle RNA.” However, the term “hairpin RNA” is also intended to encompass more complicated secondary RNA structures comprising self-annealing double stranded RNA sequences, but also internal bulges and loops. The specific secondary structure adapted will be determined by the free energy of the RNA molecule, and can be predicted for different situations using appropriate software such as FOLDRNA (Zuker and Stiegler (1981) Nucleic Acids Res 9(1):133-48; Zuker, M. (1989) Methods Enzymol. 180:262-288).

In still other embodiments of the invention, inhibition of the expression of one or more genes by RNAi may be obtained by hairpin RNA (hpRNA) interference or intron-containing hairpin RNA (ihpRNA) interference. For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene product whose expression is to be inhibited, and an antisense sequence that is fully or partially complementary to the sense sequence. Alternatively, the base-paired stem region may correspond to a portion of a promoter sequence controlling expression of the gene encoding the target polypeptide to be inhibited. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk et al. (2002) Plant Physiol. 129:1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407:319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407:319-320; Wesley et al. (2001) Plant J 27:581-590; Wang and Waterhouse (2001) Curr. Opin. Plant Biol. 5:146-150; Waterhouse and Helliwell (2003) Nat. Rev. Genet. 4:29-38; Helliwell and Waterhouse (2003) Methods 30:289-295, and U.S. Patent Publication No. 20030180945, each of which is herein incorporated by reference.

By “encoding” is meant that a nucleic acid sequence or part(s) thereof corresponds, by virtue of the genetic code of an organism in question, to a particular amino acid sequence, e.g., the amino acid sequence of a desired polypeptide or protein. By means of example, nucleic acids “encoding” a particular polypeptide or protein, e.g. an enzyme, may encompass genomic, hnRNA, pre-mRNA, mRNA, cDNA, recombinant or synthetic nucleic acids.

The terms “polypeptide” and “protein” are used interchangeably herein and generally refer to a polymer of amino acid residues linked by peptide bonds, and are not limited to a minimum length of the product. Thus, peptides, oligopeptides, polypeptides, dimers (hetero- and homo-), multimers (hetero- and homo-), and the like, are included within the definition. Both full-length proteins and fragments thereof are encompassed by the definition. The terms also include post-expression modifications of the polypeptide, for example, glycosylation, acetylation, phosphorylation, etc. Furthermore, for purposes of the present invention, the terms also refer to such when including modifications, such as deletions, additions and substitutions (e.g., conservative in nature), to the sequence of a native protein or polypeptide.

The term “variant” or “homolog” when used in connection to a protein, such as an enzyme, for example as in “a variant of protein X”, refers to a protein, such as an enzyme, that is altered in its sequence compared to protein X, but that retains the activity of protein X, such as the enzymatic activity (i.e. a functional variant or homolog).

As used herein, the term “homolog” or “homologous” with regard to a contiguous nucleic acid sequence refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.”

As used herein, a culture, an in particular an algal cell culture may be in a bioreactors, an laboratory or industrial setting, or an external setting, such as a pond or other appropriate location for the growth of algae.

A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most cell or tissue types.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that responds to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone are general examples (Schena et al. (1991) Proc. Natl. Acad. Sci. USA 88:0421).

In one embodiment, the upstream region, or promoter, of the nitrate reductase (NR) gene may be used to control expression of heterologous genes in algae. As has been previously shown, some algae can adsorb nitrate and convert it into ammonium with the help of (NR). As such, it has been shown that expression of the nitrate reductase is switched off when cells are grown in the presence of ammonium ions and becomes switched on within 4 h when cells are transferred to a medium containing nitrate. In one preferred embodiment, a Chlamydomonas nitrate reductase promoter may be specifically used as an inducible promoter to control expression of heterologous polynucleotides in algae as herein described, such expression being controlled by the presence or absence of light, nitrate, or ammonium.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into, such as a bacteria.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

The term “sequence identity” or “identity,” as used herein in the context of two nucleic acid or polypeptide sequences, refers to the residues in the two sequences that are the same when aligned for maximum correspondence over a specified comparison window.

As used herein, the term “percentage of sequence identity” may refer to the value determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

An “expression vector” or “vector” is nucleic acid capable of replicating in a selected host cell or organism. An expression vector can replicate as an autonomous structure, or alternatively, in a preferred embodiment, can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assist in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table 1a, infra, contains information about which nucleic acid codons encode which amino acids. in additional, any reference to a codon, includes optimized codons.

Amino acid Nucleic acid codons

| Amino Acid | Nucleic Acid Codons |
|---|---|
| Ala/A | GCT, GCC, GCA, GCG |
| Arg/R | CGT, CGC, CGA, CGG, AGA, |
| | AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983).

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed or not expressed at all.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise. For example, the term “a bacterium” includes both a single bacterium and a plurality of bacteria.

As used here “suppression” or “silencing” or “inhibition” are used interchangeably to denote the down-regulation of the expression of the product of a target sequence relative to its normal expression level in a wild-type organism. Suppression includes expression that is decreased by about 10%, 15%, 20%, 25%, 30%, 35%, 40%, 45%, 50%, 55%, 60%, 65%, 70%, 75%, 80%, 85%, 90%, 95%, or 100% relative to the wild-type expression level. An “effective amount” is an amount of inhibitory RNA sufficient to result in suppression or inhibition of a plant pathogen.

A “host cell” is a cell which contains an introduced nucleic acid construct and supports the replication and/or expression of the construct.

Polynucleotide sequences may have substantial identity, substantial homology, or substantial complementarity to the selected region of the target gene. As used herein “substantial identity” and “substantial homology” indicate sequences that have sequence identity or homology to each other. Generally, sequences that are substantially identical or substantially homologous will have about 75%, 80%, 85%, 90%, 91%, 92%, 93%, 94%, 95%, 96%, 97%, 98%, 99%, or 100% sequence identity wherein the percent sequence identity is based on the entire sequence and is determined by GAP alignment using existing default parameters (GCG, GAP version 10, Accelrys, San Diego, Calif.). GAP uses the algorithm of Needleman and Wunsch ((1970) J Mol Biol 48:443-453) to find the alignment of two complete sequences that maximizes the number of matches and minimizes the number of sequence gaps. Sequences which have 100% identity are identical. “Substantial complementarity” refers to sequences that are complementary to each other, and are able to base pair with each other. In describing complementary sequences, if all the nucleotides in the first sequence will base pair to the second sequence, these sequences are fully complementary.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise. For example, the term “a microorganism” includes both a single microorganism and a plurality of microorganisms.

As used here “suppress,” “suppression” or “silencing” or “inhibition” are used interchangeably to denote the down-regulation of the expression of the product of a target sequence relative to its normal expression level in a wild-type organism. Suppression includes expression that is decreased by about 10%, 15%, 20%, 25%, 30%, 35%, 40%, 45%, 50%, 55%, 60%, 65%, 70%, 75%, 80%, 85%, 90%, 95%, or 100% relative to the wild-type expression level. An “effective amount” is an amount of inhibitory RNA sufficient to result in suppression or inhibition of a plant pathogen. The term modulate may denote the up or down-regulation of the expression of the product of a target sequence relative to its normal expression level in a wild-type organism.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Overexpression of Jojoba Wax Synthase (WS) and Fatty Acyl-CoA Reductase (FAR) in Chlamydomonas

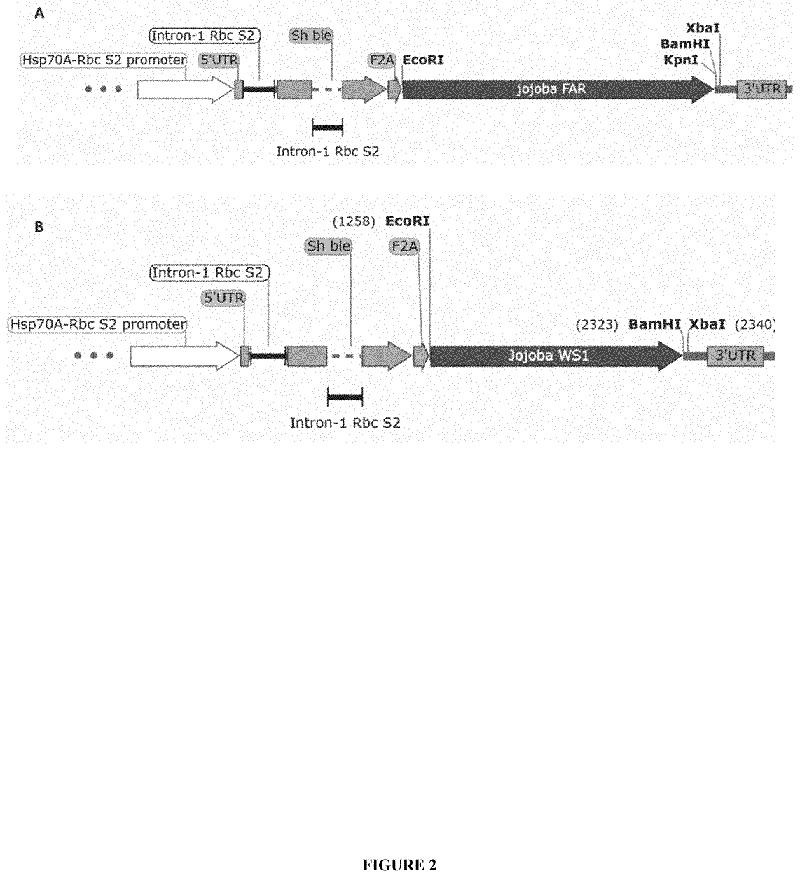

In one embodiment, Jojoba wax synthase (jjWS1) and fatty acyl-CoA reductase (jjFAR) genes were cloned into a commercial Chlamydomonas expression vector for pChlamy_4, which features a strong hybrid constitutive promoter consisting of Hsp70 and RbcS2 promoters for strong expression of the gene of interest (Invitrogen, Thermo Fisher Scientific, USA). The FAR (SEQ ID NO. 3) and WS1 (SEQ ID NO. 4) from the desert shrub jojoba, (Simmondsia chinensis) were codon optimized for expression in Chlamydomonas and, as shown in FIG. 2, were separately cloned into the pChlamy_4 expression vector.

### Example 2: Overexpression of Euglena Fatty Acyl coA Reductase and Wax Synthase in Chlamydomonas

As noted above, in Euglena, wax esters may be produced by the esterification of fatty acyl-CoA and fatty alcohol, catalyzed by wax ester synthase or acyl-CoA:fatty alcohol acyltransferase. Enzymes exhibiting activity of wax ester synthesis have been characterized into two main groups: 1) wax synthases (WS), which exhibit only wax synthesis activity (the jojoba wax synthase JJWS1 and Euglena wax synthase EgWS1 are examples; 2) bifunctional enzymes with both wax synthase and acyl-CoA:diacylglycerol acyltransferase (DGAT) activities (WSDs), utilizing a broad range of acyl-CoAs and fatty alcohols from C12 to C20 in length. In Euglena, WSD2 and WSD5 have been shown to exhibit wax ester formation in vivo.

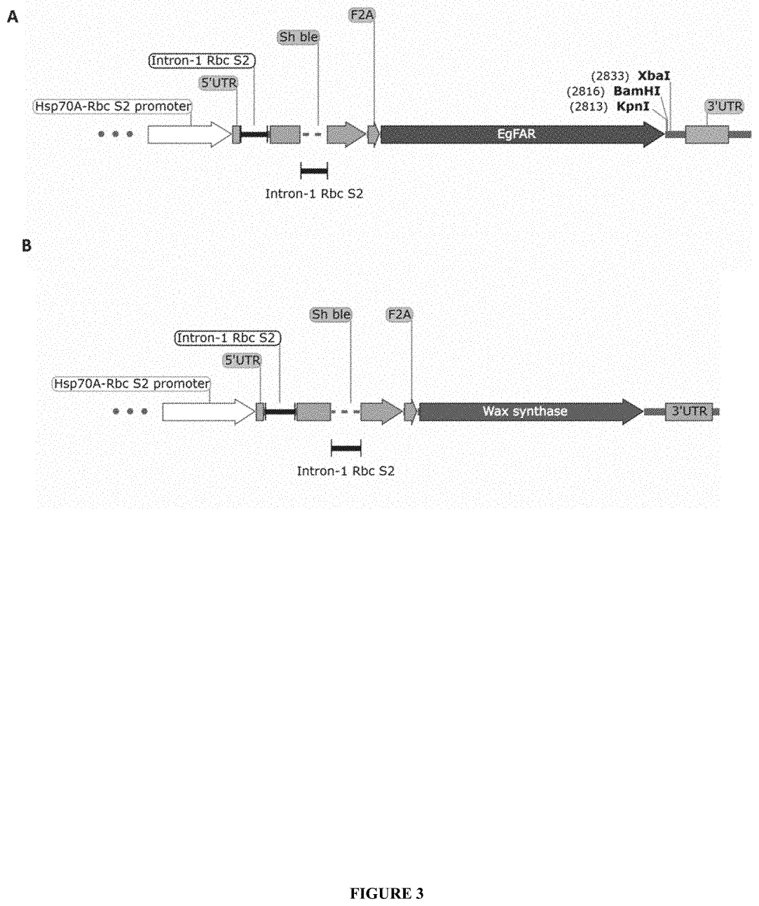

As shown in FIG. 3, the present inventors tested both types of wax synthases. Specifically, gene cassettes of Euglena gracilis fatty acyl-CoA reductase (A) and wax synthase (B) were cloned into a pChlamy_4 expression vector, driven by the Hsp 70A-Rbc S2 promoter, a strong hybrid constitutive promoter consisting of Hsp70 and RbcS2 promoters. As noted above, the synthase (B) can be a wax synthase (WS1) or a dual enzyme with wax synthase and acyl-CoA:diacylglycerol acyltransferase (DGAT). For inducible gene expression, the present inventors generated a modified version of the expression vector pSL18 was used, where the paromomycin resistance marker gene was replaced by zeocin resistance marker gene and the PSAD promoter was replaced by either the nitrate-inducible NIT1 promoter or the copper-inducible CYC6 promoter.

### Example 3: Generation of Wax Esters in C. reinhardtii

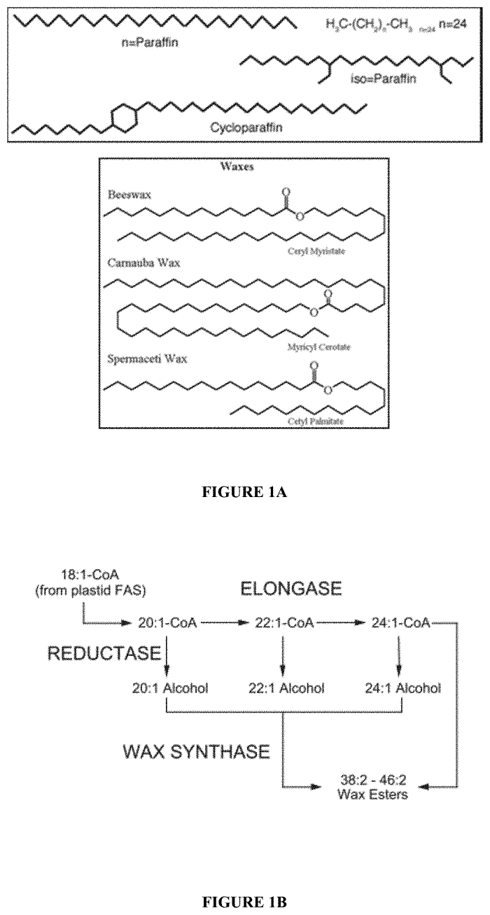

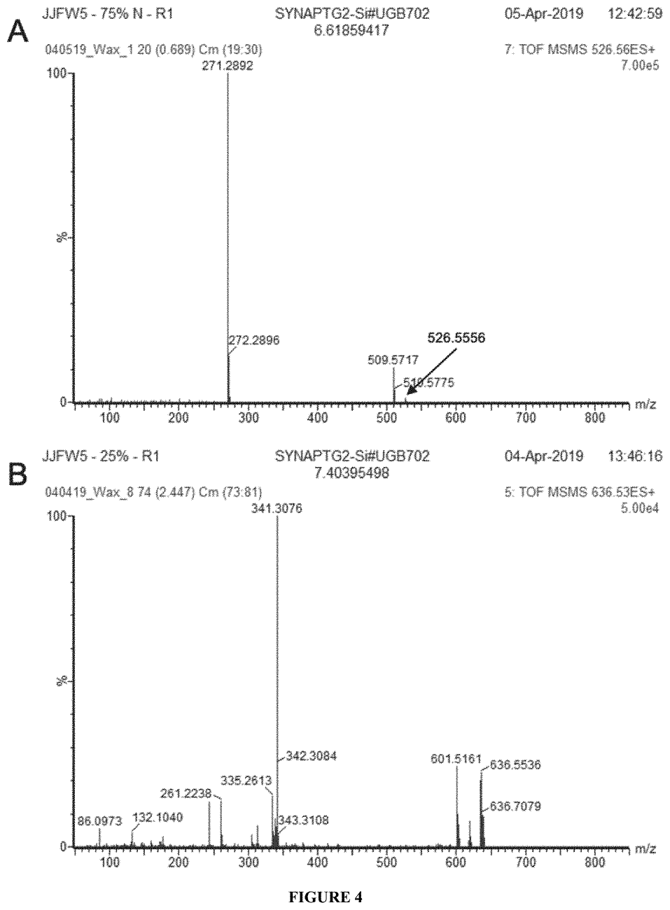

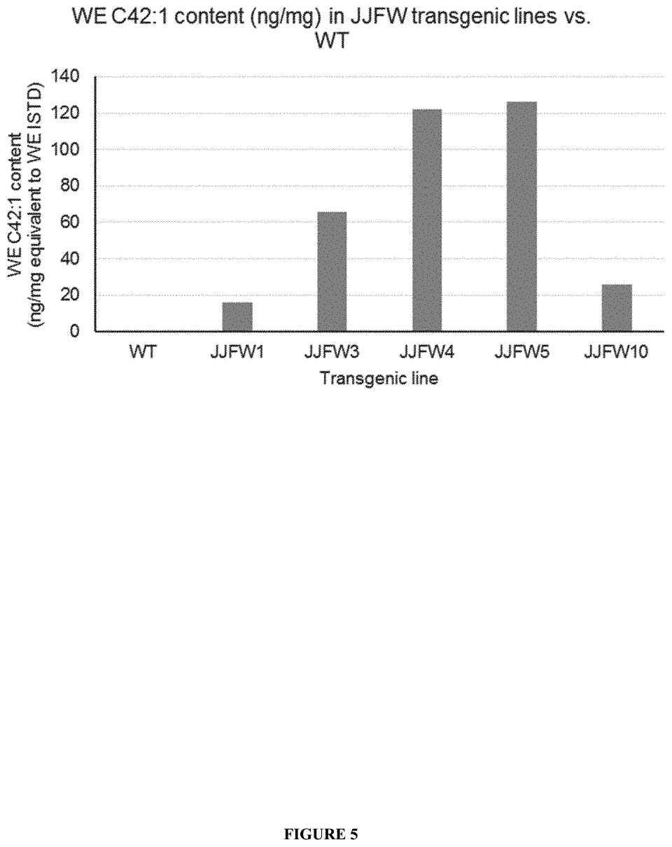

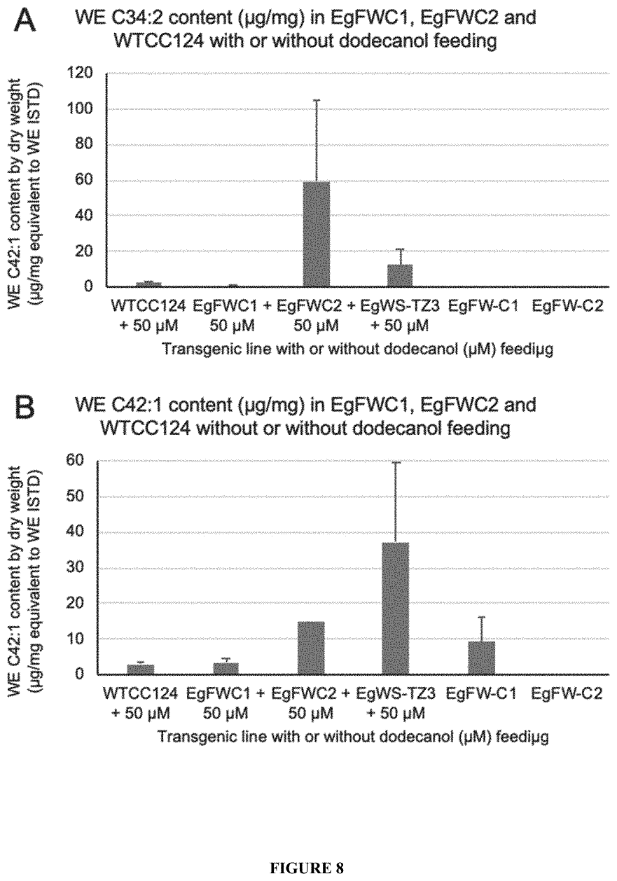

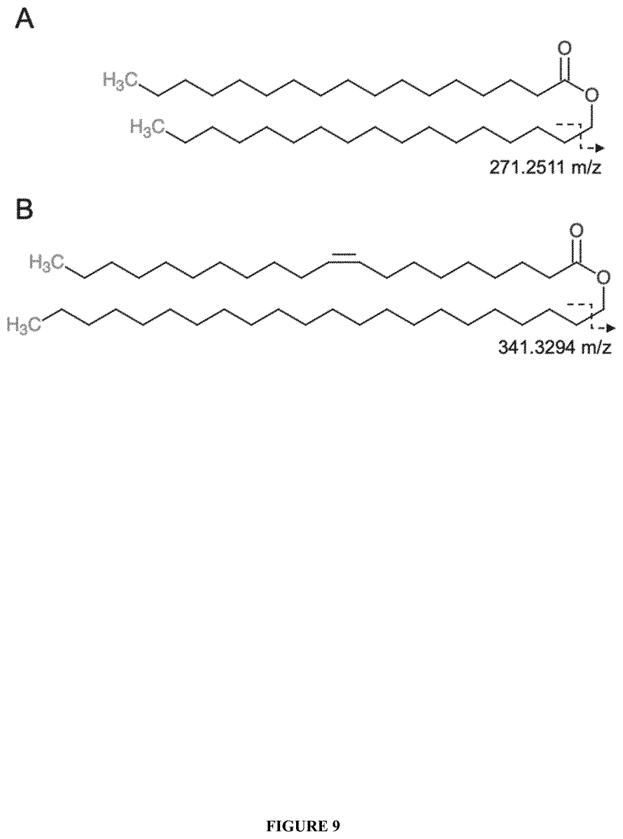

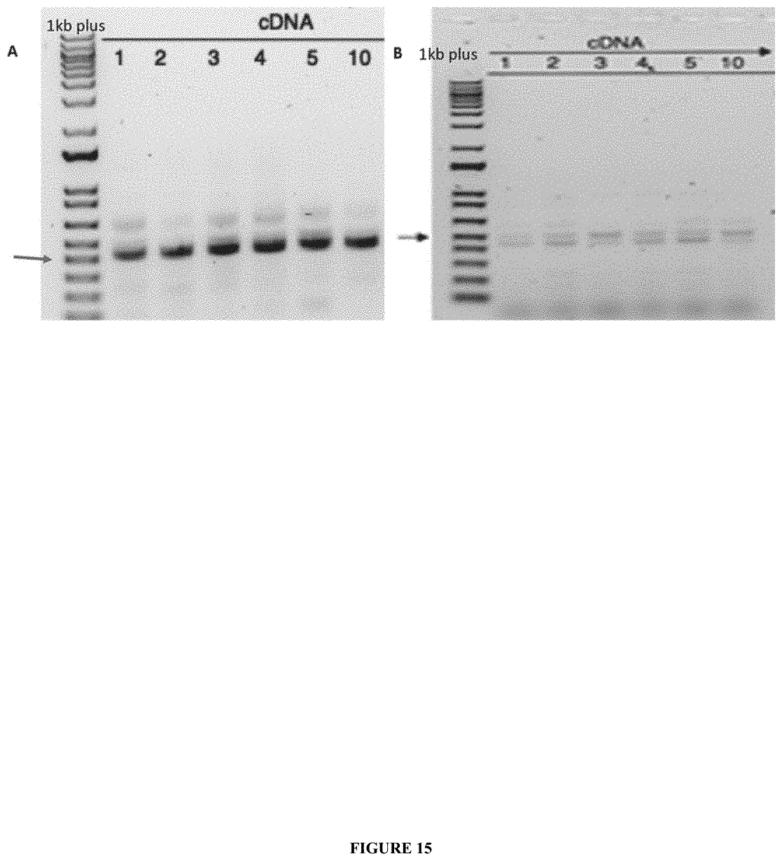

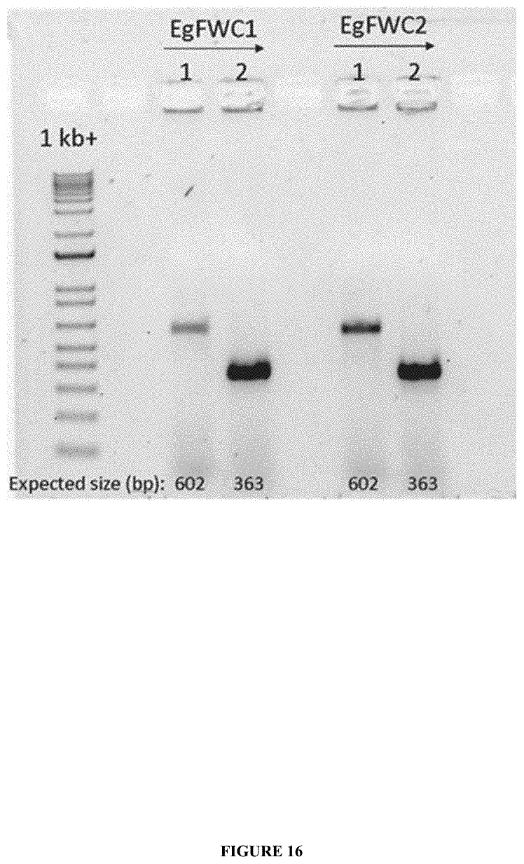

The present inventors demonstrated the transformation and heterologous overexpression of the expression cassettes identified in FIG. 2. Specifically, present inventors demonstrated the transformation and heterologous overexpression the jojoba wax biosynthesis genes, specifically jojoba fatty acyl-CoA reductase (jjFAR) (SEQ ID NO. 3), and jojoba wax synthase (jjWS1) (SEQ ID NO. 4) in the algal species Chlamydomonas. As shown in FIGS. 4, 15 and 9, this heterologous expression of jjFAR and jjWS1 resulted in the production of a C42:1 wax species, with an acyl species identity of C20:1/C22:0. This species was not detected in the wild-type. The identified ester was consistent with esters produced in the plant as outlined in FIG. 1. As further shown in FIG. 5, wax ester yield was highest in the transgenic lines JJFW4 and JJFW5 heterologously expressing jjFAR (SEQ ID NO. 1) and jjWS1 (SEQ ID NO. 2) proteins. As further demonstrated by the present inventors in FIG. 16, two transgenic lines overexpressing Euglena fatty acyl-CoA reductase (FAR) and wax synthase (WS1). EgFWC2 produced the C42:1 wax species and additionally the C34:2 after feeding with dodecanol (See FIG. 8).

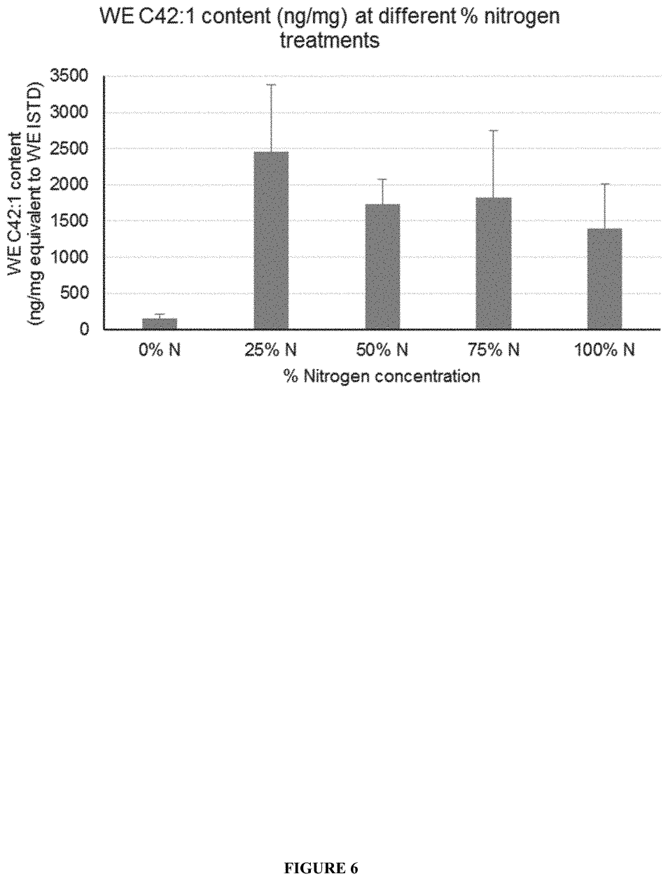

The present inventors used recombinant C. reinhardtii strain JJFW5 in a preliminary experiment to investigate the effect of nitrogen starvation on wax ester biosynthesis. The cultures were spun down after 5 days of growth on normal TAP media with nitrate. The pellet was resuspended and incubated for an additional 48 hours in TAP medium without reduced nitrogen (0%, 25%, 50%, 75% and 100%). As generally shown in FIG. 6, yield increased by up to 75% were seen in a reduced nitrogen growth. Complete nitrogen removal for 48 hours was detrimental to culture growth and resulted in the lowest wax ester yield.

### Example 4: Enhancing Algal Biomass for Improved Wax Ester Yields

The present inventors utilized inducible promoters to redirect carbon flow from biomass to wax ester production. Increasing biomass productivity prior to induction of wax ester biosynthesis may enhance yield of wax esters. To increase biomass, the present inventors overexpressed the dual cyanobacterial fructose-1,6-/sedoheptulose-1,7-bisphosphatase (fII) according to according to peptide sequence SEQ ID NO. 24, and nucleotide sequence SEQ ID NO. 25). Overexpression of III may increase photosynthesis and growth leading to enhanced production of wax esters

### Example 5: Feeding Fatty Alcohols to Generate Unique Wax Esters

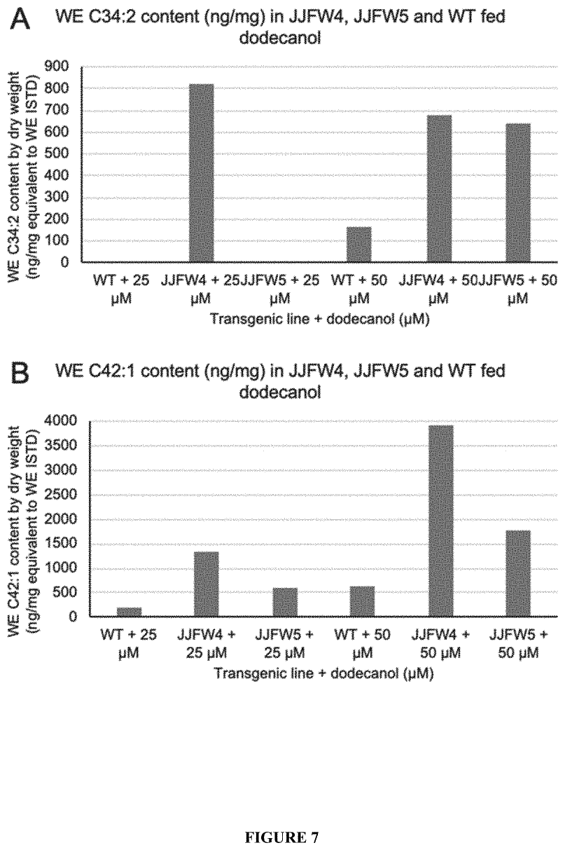

As shown in FIG. 7, the present inventors demonstrate that when transgenic C. reinhardtii strains overexpressing jjFAR (SEQ ID NO. 3) and jjWS1 (SEQ ID NO. 4) were fed with 1-dodecanol (C₁₂H₂₆O), one additional C34:2 wax ester not seen in unfed cultures was detected. These data demonstrate that feeding synthetic alcohols to recombinant algal strains, such as described herein, is a feasible and cost-effective strategy for generating unique waxes. Feeding 1-dodecanol to transgenic lines overexpressing Euglena genes, resulted in the production of the C42:1 and C34:2 wax ester species (FIG. 8). This species is not detected in unfed transgenic lines overexpressing Euglena Fatty acyl-CoA reductase (egFAR) (SEQ ID NO. 7), and wax ester synthase (egWS1) (SEQ ID NO. 8). Moreover, The C34:2 species was only detected in cultures fed with 1-dodecanol.

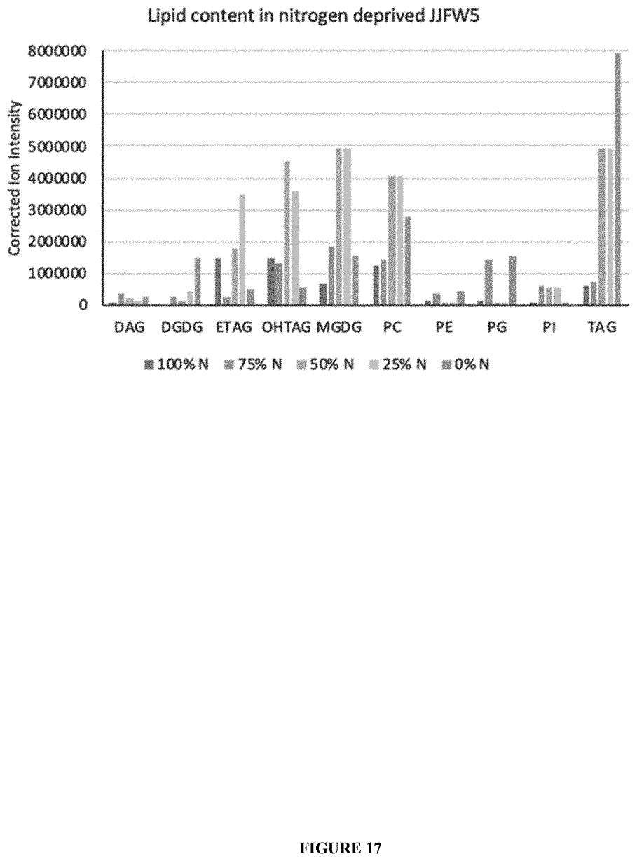

Lipid profiles were generated to determine potential bottlenecks in wax ester production. As shown in FIG. 17, lipid production during nitrogen deprivation revealed increased lipid productivity at 0, 25 and 50% nitrogen (N) content. In particular, triacylglycerol (TAG) production was 5-fold higher at 25 and 50% N and 8-fold higher at 0% N. Phosphatidylcholine (PC) and monogalactosyl diacylglycerol (MGDG) were approximately 4- and 5-fold higher in cultures with either 25 or 50% N vs. 100% N, and approximately 2- and 3-fold higher in cultures with 0% N vs. 100% N. Other phospholipid species were detected at much lower levels than PC, likely due to poor ionization in positive mode relative to PC.

In addition, epoxy- and hydroxy-triacylglycerol species (ETAG, OHTAG) were observed, with the highest levels occurring in cultures with 25 and 50% N. Epoxy fatty acids are synthesized by lipooxygenases or peroxygenases, while hydroxy fatty acids are synthesized by fatty acid hydroxylases or early termination of fatty acid elongation. ETAG and OHTAG may also be formed from TAG by oxidation; however, oxidation of TAG to ETAG and OHTAG was shown to occur at higher temperatures (70° C.), while species were shown to be stable at 40° C. for at least 10 days. All sample preparation and extraction steps were performed at either 4° C. or room temperature.

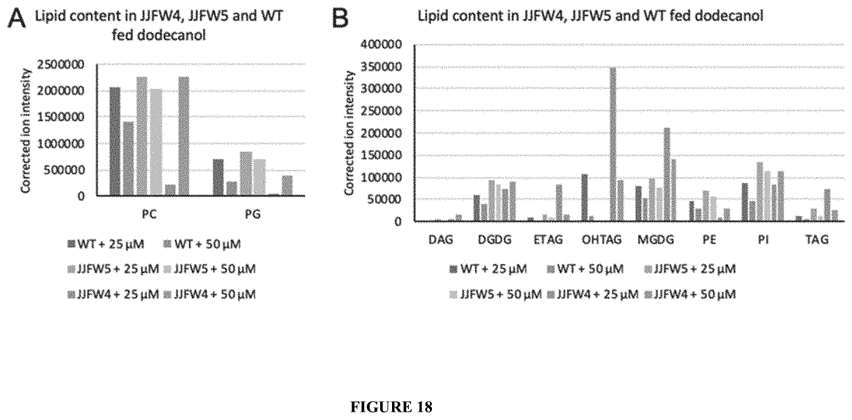

As demonstrated in FIG. 18, both epoxy and hydroxylated triacylglycerol species (ETAG, OHTAG) were also observed in dodecanol fed WT and transgenic cultures. Epoxy and hydroxy fatty acids are components of various plant waxes. Hydroxy esters are produced from hydroxy fatty acids and primary fatty alcohols, and are a component found in beeswax. Hydroxy triacylglycerols (OHTAG) were identified in C. reinhardtii WT and transgenic lines, demonstrating that the algae are synthesizing hydroxy fatty acids. Notably, this aspect of the invention creates the potential for novel wax blends with different properties for numerous applications.

Notably, primary fatty alcohols are required for wax ester synthesis; therefore the fatty acid reductases (FAR) selected for wax ester synthesis in C. reinhardtii should be specific to produce primary fatty alcohols over secondary fatty alcohols or fatty aldehyde intermediates. FARs that produce C16:0-C26:0 and C24:0-C30:0 primary fatty alcohols directly from fatty acids have been previously identified and characterized in Arabidopsis.

### Example 6: Materials and Methods

Cultivation and transformation of algae. C. reinhardtii wild-type strain CC124 was used as the background strain in all our experiments. Cells were grown in TAP (Tris-acetate-phosphate) medium (Gorman and Levin, 1965) at 23 degrees C. under constant illumination in shaking culture flasks. Transformation was done by electroporation following the GeneArt® MAX Efficiency® Transformation protocol (Invitrogen, Thermo Fisher Scientific, USA).

RT-PCR analysis. Expression of transgene was confirmed in 3-5 day-old algae cultures growing in TAP media by RT-PCR. For RT-PCR analysis, a pellet from 2 mL of algae culture was frozen in liquid nitrogen and ground in a TissueLyser (QIAGEN Inc, USA). RNA was extracted following the EZNA plant RNA extraction kit (Omega Bio-tek Inc, USA). Up to a microgram of total RNA was used to synthesize cDNA using the superscript III cDNA synthesis kit (Thermo Fisher Scientific, USA). The cDNA was used to check for the expression of transgenes by RT-PCR.

Sample Preparation. Algal biomass was collected by centrifugation at 2500 rpm×10 minutes×4° C., and was lyophilized to dryness in a Flexi-Dry MP benchtop lyophilizer (FTS Systems, US). Sample extraction was performed following Iven et al. 2015 with a few modifications. Approximately 20-30 mg of dried algal biomass was weighed and placed into a 2 mL centrifuge tube. 0.25 mL equivalent of 0.5 mm glass beads and 1 mL of chloforom:methanol (1:1 v/v) were added to the centrifuge tubes. Samples were homogenized for 15 minutes in a TissueLyzer LT (Qiagen, US) at 50 oscillations/second. Cell debris were cleared by centrifugation (15,000 rpm×2 minutes×25° C.), and 0.7 mL supernatant were transferred to a fresh 2 mL centrifuge tube. Algal biomass samples were re-extracted with 1 mL of n-hexane:diethyl ether:glacial acetic acid (80:20:0.1 v/v/v), and homogenized with the TissueLyzer at 50 oscillations/second for additional 5 minutes. Cell debris were cleared by centrifugation again, and 0.8 mL supernatant were combined with the previous extract. Samples were dried down in a SpeedVac SC110 (Savant, US) for 1 hr, and then resuspended in 0.3 mL chloroform:methanol (1:1 v/v). Samples were diluted 375-fold in 90:10 isopropanol:methanol with 10 mM ammonium acetate and approximately 10 nmol/mL WE C34:0 (17:0/17:0) as an internal standard.

ESI-MS/MS. Wax ester samples were infused at 1.0 μL/min through the sample fluidics syringe pump of the Synapt G2-Si (Waters, US). A lockmass solution of 200 pmol/μL leucine enkephalin was infused at 5.0 μL/min through the lockspray fluidics syringe pump during the analysis. Wax esters were detected in positive ionization mode (+ES) with a capillary and cone voltage of 3.0 K and 40 V, respectively. Source and desolvation temperatures were 100° C. and 200° C., respectively, and desolvation and nebulizer gas flows were set to 650 L/Hr and 6.5 bar, respectively. A data-dependent acquisition (DDA) method was used to obtain lipid and wax ester profiles. MS survey data were acquired in resolution mode, over a mass range of 300-1000 m/z with a 0.6 s scan time and 14 ms interscan delay. MS/MS was triggered when the signal intensity of an individual ion rose above 5000, and data were collected for a mass range of 50-850 m/z using a 0.2 s scan time and 14 ms interscan delay. MS/MS was switched back to MS survey when the signal intensity of an individual ion dropped below 1000 or after 2.0 s regardless. MS and MS/MS data were collected in continuum mode, and lockmass data were acquired for 1.0 s every 10 s during the acquisition. Real-time exclusion was applied to acquire data for a given ion once and then exclude for the remaining run time, with an exclusion window of ±200 mDa. An inclusion list was used to assign priority to acquire masses included on the list. The inclusion list was generated from wax ester species observed in Euglena gracilis and jojoba oil (Lassner et al., 1999; Tomiyama et al., 2017). The total run time of the analysis was 5 minutes.

Data processing. Accurate mass measurement correction was applied to raw signal intensities in MassLynx 4.2 (Waters, US) using leucine enkephalin (556.2771 m/z). For wax ester semi-quantitation, corrected intensities were used to calculate calibration factors (CF), calibration response factors (CRF), generate calibration curves and approximate ng/mg concentration of wax esters in Microsoft Excel. For wax ester species where analytical standards were not available, a wax ester standard of similar composition and the same prototype group was used to generate a calibration curve for semi-quantitation.

CRF=[(WE ISTD corrected intensity)×(WE species STD (nmol/mL))/(WE ISTD concentration (nmol/mL)×(WE species STD corrected intensity)) Equation 1:

nmol/mL=[(WE species corrected intensity)×(WE ISTD nmol/mL)]/[(WE ISTD corrected intensity)×(CRF)] Equation 2:

ng/mg=[(WE species nmol/mL)×(Sample volume mL)×(Dilution Factor)×(WE species ng/nmol)]/(mg dried algal biomass) Equation 3:

Lipid profiles were generated in LipidXplorer V1.2.6 (#ref5). After accurate mass measurement correction, Waters .RAW files were converted to .mzML files using MSConvert (#ref6). The .mzML files were imported into LipidXplorer, with the following import settings: a selection window of 0.2 Da, time range of 300 s, MS mass range of 300-1000 m/z, MS/MS mass range of 50-850 m/z, MS and MS/MS resolution of 20000 and 15000 FMHW, respectively, and a tolerance of 100 ppm for MS and MS/MS. MFQL files were created for 14 lipid species with ammonium adducts analyzed in +ES. Results in the output (.csv) file included mass, species name, acyl species assignment, chemical formula, error (ppm), precursor intensity, and product ion intensity.

### Tables

TABLE 1
List of wax ester species, molecular formula, molecular
weight (g/mol), molecular formula with ammonium
adduct, masses used for inclusion list (m/z).

| Wax Ester | Molecular | | Adduct | |
|---|---|---|---|---|
| species | Formula | MW (g/mol) | [M + NH4]+ | m/z |
| C22:0 | C22H44O2 | 340.3300 | C22H48N1O2 | 358.3685 |
| C24:0 | C24H48O2 | 386.3998 | C24H52N1O2 | 386.3998 |
| C26:0 | C26H52O2 | 396.3926 | C26H56N1O2 | 414.4311 |
| C28:0 | C28H56O2 | 424.4239 | C28H60N1O2 | 442.4624 |
| C30:0 | C30H60O2 | 452.4552 | C30H64N1O2 | 470.4937 |
| C32:0 | C32H64O2 | 480.4865 | C32H68N1O2 | 498.5250 |
| C32:1 | C32H62O2 | 478.4709 | C32H66N1O2 | 496.5094 |
| C34:0 | C34H68O2 | 508.5217 | C34H72N1O2 | 526.5602 |
| C34:1 | C34H66O2 | 506.5022 | C34H70N1O2 | 524.5407 |
| C34:2 | C34H64O2 | 504.4865 | C34H68N1O2 | 522.5250 |
| C36:0 | C36H72O2 | 536.5530 | C36H76N1O2 | 554.5915 |
| C36:1 | C36H70O2 | 534.5373 | C36H74N1O2 | 552.5758 |
| C36:2 | C36H68O2 | 532.5217 | C36H72N1O2 | 550.5602 |
| C38:0 | C38H76O2 | 564.5843 | C38H80N1O2 | 582.6228 |
| C38:1 | C38H74O2 | 562.5686 | C38H78N1O2 | 580.6071 |
| C38:2 | C38H72O2 | 560.5530 | C38H76N1O2 | 578.5915 |
| C40:0 | C40H80O2 | 592.6156 | C40H84N1O2 | 610.6541 |
| C40:1 | C40H78O2 | 590.5999 | C40H82N1O2 | 608.6384 |
| C40:2 | C40H76O2 | 588.5843 | C40H80N1O2 | 606.6228 |
| C42:0 | C42H84O2 | 620.6469 | C42H88N1O2 | 638.6854 |
| C42:1 | C42H82O2 | 618.6312 | C42H86N1O2 | 636.6697 |
| C42:2 | C42H80O2 | 616.6156 | C42H84N1O2 | 634.6541 |
| C44:0 | C44H88O2 | 648.6782 | C44H92N1O2 | 666.7167 |
| C44:1 | C44H86O2 | 646.6625 | C44H90N1O2 | 664.7010 |
| C44:2 | C44H84O2 | 644.6469 | C44H88N1O2 | 662.6854 |
| C46:0 | C46H92O2 | 676.7095 | C46H96N1O2 | 694.7480 |
| C46:1 | C46H90O2 | 674.6938 | C46H94N1O2 | 692.7323 |
| C46:2 | C46H88O2 | 672.6782 | C46H92N1O2 | 690.7167 |

TABLE 2
Exemplary fatty acid reductase 1 (Arabidopsis thaliana)

| Gene | Arabidopsis loci |
|---|---|
| FAR1 | At5g22500 |
| FAR2 (MR2) | At3g11980 |
| FAR3 (CER4) | At4g33790 |
| FAR4 | At3g44540 |
| FAR5 | At3g44550 |
| FAR6 | At3g56700 |
| FAR7 | At5g22420 |
| FAR8 | At3g44560 |

TABLE 3
Exemplary wax synthase (Arabidopsis thaliana)

| Gene | Arabidopsis loci |
|---|---|
| WSD1 | At5g37300 |
| WSD2 | At1g72110 |
| WSD3 | At2g38995 |
| WSD4 | At3g49190 |
| WSD5 | At3g49200 |
| WSD6 | At3g49210 |
| WSD7 | At5g12420 |
| WSD8 | At5g16350 |
| WSD9 | At5g22490 |
| WSD10 | At5g53380 |
| WSD11 | At5g53390 |

### REFERENCES

The following references are incorporated into the specification in their entirety:
[1] Cook T (2010) LW-10-107. Nat. Petrochem. & Refiner Assoc.
[2] Crisp, S., Eaton, R. F., & Tinsley, H. M. (1984). Scheme for the identification of sperm whale oil and its products in commercial formulations and in leather articles. The Analyst, 109(November), 1497-1502.
[3] Koblenz, B., & Lechtreck, K. (2005). The NIT1 Promoter Allows Inducible and Reversible Silencing of Centrin in Chlamydomonas reinhardtii, 4(11), 1959-1962.
[4] Lassner, M. W., Lardizabal, K., & Metz, J. G. (1999). Producing wax esters in transgenic plants by expression of genes derived from jojoba. Perspectives on New Crops and New Uses, 220-224.
[5] Quinn, J. M., Kropat, J., & Merchant, S. (2003). Copper Response Element and Crr1-Dependent Ni 2 [symbol] -Responsive Promoter for Induced, Reversible Gene Expression in Chlamydomonas reinhardtii, 2(5), 995-1002.
[6] Řezanka, T., & Podojil, M. (1986). Identification of wax esters of the fresh-water green alga Chlorella kessleri by gas chromatography-mass spectrometry. Journal of Chromatography A, 362(C), 399-406.
[7] Teerawanichpan, P., & Qiu, X. (2010). Fatty Acyl-CoA Reductase and Wax Synthase from Euglena gracilis in the Biosynthesis of Medium-Chain Wax Esters, 263-273.
[8] Tomiyama, T., Kurih, K., Ogawa, T., & Maruta, T. (2017). Wax Ester Synthase/Diacylglycerol Acyltransferase Isoenzymes Play a Pivotal Role in Wax Ester Biosynthesis in Euglena gracilis, (October), 1-13.
[9] Tucci, S., Vacula, R., Krajcovic, J., Proksch, P., & Martin, W. (2010). Variability of wax ester fermentation in natural and bleached Euglena gracilis strains in response to oxygen and the elongase inhibitor flufenacet. Journal of Eukaryotic Microbiology, 57(1), 63-69.
[10] Vanhercke, T., Wood, C. C., Green, A. G., Vanhercke, T., Wood, C. C., Stymne, S., Green, A. G. (2012). Metabolic engineering of plant oils and waxes for use as industrial feedstocks Metabolic engineering of plant oils and waxes for use as industrial feedstocks, (NOVEMBER).
[11] Iven, Tim, et al. “Wax ester profiling of seed oil by nano-electrospray ionization tandem mass spectrometry.” Plant Methods 9.1 (2013): 24.
[12] Chen, J., Green, K. B., & Nichols, K. K. (2015). Characterization of wax esters by electrospray ionization tandem mass spectrometry: double bond effect and unusual product ions. Lipids, 50(8), 821-836.
[13] Herzog, R., Schwudke, D., & Shevchenko, A. (2013). LipidXplorer: software for quantitative shotgun lipidomics compatible with multiple mass spectrometry platforms. Current protocols in bioinformatics, 43(1), 14-12.
[14] Chambers, M. C., Maclean, B., Burke, R., Amodei, D., Ruderman, D. L., Neumann, S., & Hoff, K. (2012). A cross-platform toolkit for mass spectrometry and proteomics. Nature biotechnology, 30(10), 918.
[15] Quinn J M, Kropat J, Merchant S. Copper Response Element and Crr1-Dependent Ni2+-Responsive Promoter for Induced, Reversible Gene Expression in Chlamydomonas reinhardtii. Eukaryotic Cell. 2003; 2(5):995-1002.
[16] Ferrante P, Catalanotti C, Bonente G, Giuliano G. An Optimized, Chemically Regulated Gene Expression System for Chlamydomonas. El-Shemy H A, ed. PLoS ONE. 2008; 3(9):e3200.
[17] Doron L, Segal N, Shapira M. Transgene Expression in Microalgae—From Tools to Applications. Frontiers in Plant Science. 2016; 7:505. doi:10.3389/fpls.2016.00505.
[18] Diener D R, Curry A M, Johnson K A, et al. Rescue of a paralyzed-flagella mutant of Chlamydomonas by transformation. Proceedings of the National Academy of Sciences of the United States of America. 1990; 87(15):5739-5743.
[19] Loppes R, Radoux M (2001) Identification of short promoter regions involved in the transcriptional expression of the nitrate reductase gene in Chlamydomonas reinhardtii. Plant Mol Biol 45:215-227
[20] Craig D (2007) Int. Lubricants & Waxes Mtg, Nov. 1-2, 2007, Houston, Tex. LW-07-108
[21] Antia N J et al, (1974) J. Protozool 21: 768-771
[22] Rezanka T et al, (1986) J. Chromat. 362: 399-406
[23] Henderson R J et al, (1989) Phytochemistry 28: 1355-1361
[24] Tucci S et al, (2010) J. Eukar. Microbiol. 57: 63-69
[25] Teerawanichpan P et al, (2010) Lipids 45: 263-273
[26] Khozin-Goldberg I et al, (2010) Biochimie 93: 91-100
[27] Furuhashi T et al, (2015) Metabolomics 11: 175-183
[28] Kawabata S et al, (1990) Agric. Biol. Chem. 54: 811-812
[29] Negi S et al. (2015) J. Appl. Phycol. DOI 10.1007/s10811-015-0652-z.
[30] Guschina and Harwood (2006) Prog. Lipid Res. 45:160-187
[31] Vanhercke T et al, (2012) Plant Biotechnol. J. 11:197-210
[32] Nobusawa T et al, (2013) PLOS Biology DOI: 10.1371/journal.pbio.1001531
[33] Menendez-Bravo S et al, (2014) Metabolic Engineering 24: 97-106
[34] Advincula R C et al (2002) Langmuir 12:19
[35] Haining Ji et al, (2005) Macromolecules, 38, 9950-9956
[36] Pietaka A et al, (2007) Appl. Surf. Sci, 253, 7: 3627-3635.
[37] Vrkoslav V et al. (2009) J. Mass Spec. 44, 1: 101-110
[38] Kitaya Y et al, (2005) Adv. Space Res. 35: 1584-1588
[39] Mata T M et al, (2009) Renewable Sustainable Energy Rev. 14: 217-23223.
[40] Ogbonna J C et al, (2000) Materials Sci. and Eng. 12: 9-15
[41] Schneider T et al, (1985) Planta 166: 67-73
[42] Ryckebosch E et al, (2012) J. Oil & Fat Ind. 89: 189-198
[43] Matsuda F et al, (2011) Bioscience, Biotechnol., and Biochem. 75: 2253-2256
[44] Ferreira V S et al, (2007) Gene 389: 136-145
[45] Chacón M G et al, (2013) J. Biol. Chem. 288: 30345-30355
[46] Nakazawa M et al, (2015) Lipids 50: 483-492
[47] Kumar A et al (2013) Algal Res. 2: 321-332.
[48] Ogawa T et al, (2015) Biotechnol. Biofuels 8: 80
[49] Perrine Z et al (2012) Algal Res. 1:134-142.
[50] DeWilde, J. F.; Czopinski, C. J.; Bhan, A. ACS Catalysis 2014, 4, 4425-4433.
[51] US Provisional Patent (Application No. 62/199,669), filed Jul. 31, 2015.
[52] Guerbet, M. Compt. Rend. 1909, 149, 129.
[53] Kozlowski, J. T.; Davis, R. J. ACS Catalysis 2013, 3, 1588-1600.
[54] Batan J C et al (2013) Algal Research 2:196-203
[55] Quinn J C et al. (2012) Bioenergy Research 6:591-600
[56] Batan L et al. (2010) Environ. Sci. Technol. 44:7975-80
[57] Quinn J C et al. (2011) Bioenergy Res. 5:49-60
[58] Quinn J C et al. (2014b) Algal Res. 4:116-122
[59] Quinn J C et al., (2014a) Bioresource Technol. 171:37-43
[60] Moody J W et al., (2014) Proc. Nat. Acad. Sci. 111:23
[61] Quinn, JC and Davis R (2015) Bioresoruce Technol. 184:444-452
[62] Bennion, E P et al. (2015) Applied Energy 154:1062-1071
[63] Summers, H et al., (2015) Bioresource Technol. 196:431-440
[64] Rowland, O., Zheng, H., Hepworth S. R., Lam, P., Jetter, R., & Kunst, L. (2006). CER4 encodes an alcohol-forming fatty acyl-coenzyme A reductase involved in cuticular wax production in Arabidopsis. Plant Physiology, 142(3), 866-877.
[65] Domergue, F., Vishwanath, S. J., Joubès, J., Ono, J., Lee, J. A Bourdon. M., . . . & Rowland, O. (2010). Three Arabidopsis fatty acyl-coenzyme A reductases, FAR1, FAR4, and FAR5, generate primary fatty alcohols associated with suberin deposition. Plant physiology, 153(4), 1539-1554.
[66] Walton, T. J. (1900). Waxes, cutin and suberin. Methods in plant biochemistry, 4(11), 5-158.
[67] Kolattukudy, P. E. (1996). Biosynthetic pathways of cutin and waxes, and their sensitivity to environmental stress. Plant cuticles: an integrated functional approach.
[68] Tulloch, A. P. (1971). Beeswax: structure of the esters and their component hydroxy acids and dials. Chemistry and Physics of Lipids, 6(3), 235-265.
[69] Meesapyodsuk, D., & Qiu, X. (2011), A peroxygenase pathway involved in the biosynthesis of epoxy fatty acids in oat. Plant physiology, 157(1), 454-463.
[70] Bach, L., Michaelson, L. V., Haslam, R., Bellec, Y., Gissot, L., Marion. J., & Domergue, F. (2008). The very-long-chain hydroxy fatty acyl-CoA dehydratase PASTICCINO2 is essential and limiting for plant development. Proceedings of the National Academy of Sciences, 105(38), 14727-14731.
[71] Bates, P. D., Johnson, S. R., Cao, X., Li, J., Nam, J. W., Jaworski, J. G., & Ohlrogge, J. B. (2014). Fatty acid synthesis is inhibited by inefficient utilization of unusual fatty acids for glycerolipid assembly. Proceedings of the National Academy of Sciences, 111(3), 1204-1209.
[72] Xia, W., & Budge, S. M. (2018). Simultaneous quantification of epoxy and hydroxy fatty acids as oxidation products of triacylglycerols in edible oils. Journal of Chromatography A, 1537, 83-90.
[73] Gatignol, A., Durand, H., & Tiraby, G. (1988). Bleomycin resistance conferred by a drug-binding protein. FEBS Letters, 230(1-2), 171-175.
[74] Yan, C., & Xu, X. (2008). Bifunctional enzyme FBPase/SBPase is essential for photoautotrophic growth in cyanobacterium Synechocystis sp. PCC 6803. Progress in Natural Science, 18(2), 149-153.
[75] Ogawa, T., Tamoi, M., Kimura, A., Mine, A., Sakuyama, H., Yoshida, E., Shigeoka, S. (2015). Enhancement of photosynthetic capacity in Euglena gracilis by expression of cyanobacteria) leads to increases in biomass and wax ester production. Biotechnology for Biofuels, 1-11.
[76] Gorman D S, Levine R P (1965) Cytochrome f and plastocyanin: their sequence in the photosynthetic electron transport chain of Chlamydomonas reinhardi. Proc Natl Acad Sci USA 54: 1665-1669.

SEQUENCE IDENTIFICATION

SEQ ID NO. 1
Amino Acid
Fatty acyl-CoA reductase (jjFAR)
Simmondsia chinensis
MEEMGSILEFLDNKAILVTGATGSLAKIFVEKVLRSQPNVKKLYLLLRATDDETAALRLQNEVFGKELFK
VLKQNLGANFYSFVSEKVTVVPGDITGEDLCLKDVNLKEEMWREIDVVVNLAATINFIERYDVSLLINTY
GAKYVLDFAKKCNKLKIFVHVSTAYVSGEKNGLILEKPYYMGESLNGRLGLDINVEKKLVEAKINELQAA
GATEKSIKSTMKDMGIERARHWGWPNVYVFTKALGEMLLMQYKGDIPLTIIRPTIITSTFKEPFPGWVEG
VRTIDNVPVYYGKGRLRCMLCGPSTIIDLIPADMVVNATIVAMVAHANQRYVEPVTYHVGSSAANPMKLS
ALPEMAHRYFTKNPWINPDRNPVHVGRAMVFSSFSTFHLYLTLNFLLPLKVLEIANTIFCQWFKGKYMDL
KRKTRLLLRLVDIYKPYLFFQGIFDDMNTEKLRIAAKESIVEADMFYFDPRAINWEDYFLKTHFPGVVEH
VLN
SEQ ID NO. 2
Amino Acid
Wax synthase (jjWS1)
Simmondsia chinensis
MEVEKELKTFSEVWISAIAAACYCRFVPAVAPHGGALRLLLLLPVVLLFIFLPLRLSSFHLGGPTALYLV
WLANFKLLLFAFHLGPLSNPSLSLLHFISTTLLPIKFRDDPSNDHEKNKRTLSFEWRKVVLFVAKLVFFA
GILKIYEFRKDLPHFVISVLYCFHFYLGTEITLAASAVIARATLGLDLYPQFNEPYLATSLQDFWGRRWN
LMVSDILGLTTYQPVRRVLSRWVRLRWEVAGAMLVAFTVSGLMHEVFFFYLTRARPSWEVTGFFVLHGVC
TAVEMVVKKAVSGKVRLRREVSGALTVGFVMVTGGWLFLPQLVRHGVDLKTIDEYPVMFNYTQKKLMGLL
GW*
SEQ ID NO. 3
DNA
Fatty acyl-CoA reductase (jjFAR)-codon-optimized for expression in Chlamydomonas
Simmondsia chinensis
ATGGAGGAGATGGGCAGCATCCTGGAGTTCCTGGACAACAAGGCCATCCTGGTGACCGGCGCCACCGGCA
GCCTGGCCAAGATCTTCGTGGAGAAGGTGCTGCGCAGCCAGCCCAACGTGAAGAAGCTGTACCTGCTGCT
GCGCGCCACCGACGACGAGACCGCCGCCCTGCGCCTGCAGAACGAGGTGTTCGGCAAGGAGCTGTTCAAG
GTGCTGAAGCAGAACCTGGGCGCCAACTTCTACAGCTTCGTGAGCGAGAAGGTGACCGTGGTGCCCGGCG
ACATCACCGGCGAGGACCTGTGCCTGAAGGACGTGAACCTGAAGGAGGAGATGTGGCGCGAGATCGACGT
GGTGGTGAACCTGGCCGCCACCATCAACTTCATCGAGCGCTACGACGTGAGCCTGCTGATCAACACCTAC
GGCGCCAAGTACGTGCTGGACTTCGCCAAGAAGTGCAACAAGCTGAAGATCTTCGTGCACGTGAGCACCG
CCTACGTGAGCGGCGAGAAGAACGGCCTGATCCTGGAGAAGCCCTACTACATGGGCGAGAGCCTGAACGG
CCGCCTGGGCCTGGACATCAACGTGGAGAAGAAGCTGGTGGAGGCCAAGATCAACGAGCTGCAGGCCGCC
GGCGCCACCGAGAAGAGCATCAAGAGCACCATGAAGGACATGGGCATCGAGCGCGCCCGCCACTGGGGCT
GGCCCAACGTGTACGTGTTCACCAAGGCCCTGGGCGAGATGCTGCTGATGCAGTACAAGGGCGACATCCC
CCTGACCATCATCCGCCCCACCATCATCACCAGCACCTTCAAGGAGCCCTTCCCCGGCTGGGTGGAGGGC
GTGCGCACCATCGACAACGTGCCCGTGTACTACGGCAAGGGCCGCCTGCGCTGCATGCTGTGCGGCCCCA
GCACCATCATCGACCTGATCCCCGCCGACATGGTGGTGAACGCCACCATCGTGGCCATGGTGGCCCACGC
CAACCAGCGCTACGTGGAGCCCGTGACCTACCACGTGGGCAGCAGCGCCGCCAACCCCATGAAGCTGAGC
GCCCTGCCCGAGATGGCCCACCGCTACTTCACCAAGAACCCCTGGATCAACCCCGACCGCAACCCCGTGC
ACGTGGGCCGCGCCATGGTGTTCAGCAGCTTCAGCACCTTCCACCTGTACCTGACCCTGAACTTCCTGCT
GCCCCTGAAGGTGCTGGAGATCGCCAACACCATCTTCTGCCAGTGGTTCAAGGGCAAGTACATGGACCTG
AAGCGCAAGACCCGCCTGCTGCTGCGCCTGGTGGACATCTACAAGCCCTACCTGTTCTTCCAGGGCATCT
TCGACGACATGAACACCGAGAAGCTGCGCATCGCCGCCAAGGAGAGCATCGTGGAGGCCGACATGTTCTA
CTTCGACCCCCGCGCCATCAACTGGGAGGACTACTTCCTGAAGACCCACTTCCCCGGCGTGGTGGAGCAC
GTGCTGAAC
SEQ ID NO. 4
DNA
Wax synthase (jjWS1)-codon-optimized for expression in Chlamydomonas
Simmondsia chinensis
ATGGAGGTGGAGAAGGAGCTGAAGACCTTCAGCGAGGTGTGGATCAGCGCCATCGCCGCCGCCTGCTACT
GCCGCTTCGTGCCCGCCGTGGCCCCCCACGGCGGCGCCCTGCGCCTGCTGCTGCTGCTGCCCGTGGTGCT
GCTGTTCATCTTCCTGCCCCTGCGCCTGAGCAGCTTCCACCTGGGCGGCCCCACCGCCCTGTACCTGGTG
TGGCTGGCCAACTTCAAGCTGCTGCTGTTCGCCTTCCACCTGGGCCCCCTGAGCAACCCCAGCCTGAGCC
TGCTGCACTTCATCAGCACCACCCTGCTGCCCATCAAGTTCCGCGACGACCCCAGCAACGACCACGAGAA
GAACAAGCGCACCCTGAGCTTCGAGTGGCGCAAGGTGGTGCTGTTCGTGGCCAAGCTGGTGTTCTTCGCC
GGCATCCTGAAGATCTACGAGTTCCGCAAGGACCTGCCCCACTTCGTGATCAGCGTGCTGTACTGCTTCC
ACTTCTACCTGGGCACCGAGATCACCCTGGCCGCCAGCGCCGTGATCGCCCGCGCCACCCTGGGCCTGGA
CCTGTACCCCCAGTTCAACGAGCCCTACCTGGCCACCAGCCTGCAGGACTTCTGGGGCCGCCGCTGGAAC
CTGATGGTGAGCGACATCCTGGGCCTGACCACCTACCAGCCCGTGCGCCGCGTGCTGAGCCGCTGGGTGC
GCCTGCGCTGGGAGGTGGCCGGCGCCATGCTGGTGGCCTTCACCGTGAGCGGCCTGATGCACGAGGTGTT
CTTCTTCTACCTGACCCGCGCCCGCCCCAGCTGGGAGGTGACCGGCTTCTTCGTGCTGCACGGCGTGTGC
ACCGCCGTGGAGATGGTGGTGAAGAAGGCCGTGAGCGGCAAGGTGCGCCTGCGCCGCGAGGTGAGCGGCG
CCCTGACCGTGGGCTTCGTGATGGTGACCGGCGGCTGGCTGTTCCTGCCCCAGCTGGTGCGCCACGGCGT
GGACCTGAAGACCATCGACGAGTACCCCGTGATGTTCAACTACACCCAGAAGAAGCTGATGGGCCTGCTG
GGCTGGTAA
SEQ ID NO. 5
Amino Acid
Fatty acyl-CoA reductase (egFAR)
Euglena gracilis
MNDFYAGKGVFLTGVTGFVGKMVVEKILRSLPTVGRLYVLVRPKAGTDPHQRLHSEVWSSAGFDVVREKV
GGPAAFDALIREKVVPVPGDMVKDRFGLDDAAYRSLAANVNVIIHMAATIDFTERLDVAVSLNVLGTVRV
LTLARRARELGALHSVVHVSTCYVNSNQPPGARLREQLYPLPFDPREMCTRILDMSPREIDLFGPQLLKQ
YGFPNTYTFTKCMAEQLGAQIAHDLPFAIFRPAIIGAALSEPFPGWCDSASACGAVFLAVGLGVLQELQG
NASSVCDLIPVDHVVNMLLVTAAYTASAPPADPSPSSLALSPPQLPLATLPPGTVADVPIYHCGTSAGPN
AVNWGRIKVSLVEYWNAHPIAKTKAAIALLPVWRFELSFLLKRRLPATALSLVASLPGASAAVRRQAEQT
ERLVGKMRKLVDTFQSFVFWAWYFQTESSARLLASLCPEDRETFNWDPRRIGWRAWVENYCYGLVRYVLK
QPIGDRPPVAAEELASNRFLRAML
SEQ ID NO. 6
Amino Acid
Wax ester synthase (egWS1)
Euglena gracilis
MDFLGFPDSESERHAHFYVLASSFAAAIYMFTIPRRVKAGRKRFLLCSPVLLLNIMQPYIFFWTVGRHYC
NFIPLYAAFCTWWTAFKVMAFGIGRGPLCQFSAFHKFAVVMLLPILPHGDTNHGVKDERSGSSWSSPTYL
EMFAKFCGLGLCTYGISQLSHDGFPVLYNVFLSLIMYLHICVQYTGSNLATSKVLQVPLSDGMNQPYFST
SLSNFWGRRWNLVASSSLRHVVYDPIREGRLVPKGHPEEKPGGGKEVSRKVLGSLMAFLVSGIMHEYILW
LATGFWSGQMLLFFVVHGVAVAAERVAKVAWARHGLPAIPCAVSIPMTIGFLFGTAELLFYPPIFSANWA
EHGVADLRRQFRSLGLSV
SEQ ID NO. 7
DNA
Fatty acyl-CoA reductase (egFAR)-codon-optimized for expression in Chlamydomonas
Euglena gracilis
ATGAACGACTTCTACGCCGGCAAGGGCGTGTTCCTGACCGGCGTGACCGGCTTCGTGGGCAAGATGGTGG
TGGAGAAGATCCTGCGCAGCCTGCCCACCGTGGGCCGCCTGTACGTGCTGGTGCGCCCCAAGGCCGGCAC
CGACCCCCACCAGCGCCTGCACAGCGAGGTGTGGAGCAGCGCCGGCTTCGACGTGGTGCGCGAGAAGGTG
GGCGGCCCCGCCGCCTTCGACGCCCTGATCCGCGAGAAGGTGGTGCCCGTGCCCGGCGACATGGTGAAGG
ACCGCTTCGGCCTGGACGACGCCGCCTACCGCAGCCTGGCCGCCAACGTGAACGTGATCATCCACATGGC
CGCCACCATCGACTTCACCGAGCGCCTGGACGTGGCCGTGAGCCTGAACGTGCTGGGCACCGTGCGCGTG
CTGACCCTGGCCCGCCGCGCCCGCGAGCTGGGCGCCCTGCACAGCGTGGTGCACGTGAGCACCTGCTACG
TGAACAGCAACCAGCCCCCCGGCGCCCGCCTGCGCGAGCAGCTGTACCCCCTGCCCTTCGACCCCCGCGA
GATGTGCACCCGCATCCTGGACATGAGCCCCCGCGAGATCGACCTGTTCGGCCCCCAGCTGCTGAAGCAG
TACGGCTTCCCCAACACCTACACCTTCACCAAGTGCATGGCCGAGCAGCTGGGCGCCCAGATCGCCCACG
ACCTGCCCTTCGCCATCTTCCGCCCCGCCATCATCGGCGCCGCCCTGAGCGAGCCCTTCCCCGGCTGGTG
CGACAGCGCCAGCGCCTGCGGCGCCGTGTTCCTGGCCGTGGGCCTGGGCGTGCTGCAGGAGCTGCAGGGC
AACGCCAGCAGCGTGTGCGACCTGATCCCCGTGGACCACGTGGTGAACATGCTGCTGGTGACCGCCGCCT
ACACCGCCAGCGCCCCCCCCGCCGACCCCAGCCCCAGCAGCCTGGCCCTGAGCCCCCCCCAGCTGCCCCT
GGCCACCCTGCCCCCCGGCACCGTGGCCGACGTGCCCATCTACCACTGCGGCACCAGCGCCGGCCCCAAC
GCCGTGAACTGGGGCCGCATCAAGGTGAGCCTGGTGGAGTACTGGAACGCCCACCCCATCGCCAAGACCA
AGGCCGCCATCGCCCTGCTGCCCGTGTGGCGCTTCGAGCTGAGCTTCCTGCTGAAGCGCCGCCTGCCCGC
CACCGCCCTGAGCCTGGTGGCCAGCCTGCCCGGCGCCAGCGCCGCCGTGCGCCGCCAGGCCGAGCAGACC
GAGCGCCTGGTGGGCAAGATGCGCAAGCTGGTGGACACCTTCCAGAGCTTCGTGTTCTGGGCCTGGTACT
TCCAGACCGAGAGCAGCGCCCGCCTGCTGGCCAGCCTGTGCCCCGAGGACCGCGAGACCTTCAACTGGGA
CCCCCGCCGCATCGGCTGGCGCGCCTGGGTGGAGAACTACTGCTACGGCCTGGTGCGCTACGTGCTGAAG
CAGCCCATCGGCGACCGCCCCCCCGTGGCCGCCGAGGAGCTGGCCAGCAACCGCTTCCTGCGCGCCATGC
TGTAA
SEQ ID NO. 8
DNA
Wax ester synthase (egWS1)-codon-optimized for expression in Chlamydomonas
Euglena gracilis
ATGGACTTCCTGGGCTTCCCCGACAGCGAGAGCGAGCGCCACGCCCACTTCTACGTGCTGGCCAGCAGCT
TCGCCGCCGCCATCTACATGTTCACCATCCCCCGCCGCGTGAAGGCCGGCCGCAAGCGCTTCCTGCTGTG
CAGCCCCGTGCTGCTGCTGAACATCATGCAGCCCTACATCTTCTTCTGGACCGTGGGCCGCCACTACTGC
AACTTCATCCCCCTGTACGCCGCCTTCTGCACCTGGTGGACCGCCTTCAAGGTGATGGCCTTCGGCATCG
GCCGCGGCCCCCTGTGCCAGTTCAGCGCCTTCCACAAGTTCGCCGTGGTGATGCTGCTGCCCATCCTGCC
CCACGGCGACACCAACCACGGCGTGAAGGACGAGCGCAGCGGCAGCAGCTGGAGCAGCCCCACCTACCTG
GAGATGTTCGCCAAGTTCTGCGGCCTGGGCCTGTGCACCTACGGCATCAGCCAGCTGAGCCACGACGGCT
TCCCCGTGCTGTACAACGTGTTCCTGAGCCTGATCATGTACCTGCACATCTGCGTGCAGTACACCGGCAG
CAACCTGGCCACCAGCAAGGTGCTGCAGGTGCCCCTGAGCGACGGCATGAACCAGCCCTACTTCAGCACC
AGCCTGAGCAACTTCTGGGGCCGCCGCTGGAACCTGGTGGCCAGCAGCAGCCTGCGCCACGTGGTGTACG
ACCCCATCCGCGAGGGCCGCCTGGTGCCCAAGGGCCACCCCGAGGAGAAGCCCGGCGGCGGCAAGGAGGT
GAGCCGCAAGGTGCTGGGCAGCCTGATGGCCTTCCTGGTGAGCGGCATCATGCACGAGTACATCCTGTGG
CTGGCCACCGGCTTCTGGAGCGGCCAGATGCTGCTGTTCTTCGTGGTGCACGGCGTGGCCGTGGCCGCCG
AGCGCGTGGCCAAGGTGGCCTGGGCCCGCCACGGCCTGCCCGCCATCCCCTGCGCCGTGAGCATCCCCAT
GACCATCGGCTTCCTGTTCGGCACCGCCGAGCTGCTGTTCTACCCCCCCATCTTCAGCGCCAACTGGGCC
GAGCACGGCGTGGCCGACCTGCGCCGCCAGTTCCGCAGCCTGGGCCTGAGCGTGTAA
SEQ ID NO. 9
Amino Acid
WSD2
Euglena gracilis
MVVAETTPVANSISVGDLFWWRIDEPTNPMVISVILGMDGTISLAELRDALRPHVEDNIRLQGTPQPNGI
YSWRPYFIASVLLSLVLGWALRSLCCFSYIVAFGLLVGIALETRTGRQWRWVKVKDFALEDHIKLHVLPE
ETLECLHGFIDELASTQLPRDRAQWMVYLIHNAPGGSRILFRFHHIVGDGAGLGIWFYNLCTNAEQKKQD
MEARHELLAKSKARRAENRTKPSPLAKLDGFVSKVLLILGGTTKLLFLPRDSNSPVKGANVGKKKTAVTG
KDLLFPLEEVKHVGKALHPNITVNDTMCALVGGAFRRYYQSLHLHPEQMLMRATVPINIRPSTTAPIKME
NDFTIVFKSLPIHLPTPEERIAHFHVRMGFLKRGIEPLLSMFLQHLLTWLPEPLMRLIVLRFTICSSAVL
TNVLSSTVPFSLCGQPLTTAAFWVPTSGDIGIGISIMTYCDTVAINFIADENLIADWAPVVQFMREEWEE
MKGILGKEQHLPVMEPQKTVELVNLWRTWGFPWNTR
SEQ ID NO. 10
DNA
WSD2-codon-optimized for expression in Chlamydomonas
Euglena gracilis
ATGGTGGTGGCCGAGACCACCCCCGTGGCCAACAGCATCAGCGTGGGCGACCTGTTCTGGTGGCGCATCG
ACGAGCCCACCAACCCCATGGTGATCAGCGTGATCCTGGGCATGGACGGCACCATCAGCCTGGCCGAGCT
GCGCGACGCCCTGCGCCCCCACGTGGAGGACAACATCCGCCTGCAGGGCACCCCCCAGCCCAACGGCATC
TACAGCTGGCGCCCCTACTTCATCGCCAGCGTGCTGCTGAGCCTGGTGCTGGGCTGGGCCCTGCGCAGCC
TGTGCTGCTTCAGCTACATCGTGGCCTTCGGCCTGCTGGTGGGCATCGCCCTGGAGACCCGCACCGGCCG
CCAGTGGCGCTGGGTGAAGGTGAAGGACTTCGCCCTGGAGGACCACATCAAGCTGCACGTGCTGCCCGAG
GAGACCCTGGAGTGCCTGCACGGCTTCATCGACGAGCTGGCCAGCACCCAGCTGCCCCGCGACCGCGCCC
AGTGGATGGTGTACCTGATCCACAACGCCCCCGGCGGCAGCCGCATCCTGTTCCGCTTCCACCACATCGT
GGGCGACGGCGCCGGCCTGGGCATCTGGTTCTACAACCTGTGCACCAACGCCGAGCAGAAGAAGCAGGAC
ATGGAGGCCCGCCACGAGCTGCTGGCCAAGAGCAAGGCCCGCCGCGCCGAGAACCGCACCAAGCCCAGCC
CCCTGGCCAAGCTGGACGGCTTCGTGAGCAAGGTGCTGCTGATCCTGGGCGGCACCACCAAGCTGCTGTT
CCTGCCCCGCGACAGCAACAGCCCCGTGAAGGGCGCCAACGTGGGCAAGAAGAAGACCGCCGTGACCGGC
AAGGACCTGCTGTTCCCCCTGGAGGAGGTGAAGCACGTGGGCAAGGCCCTGCACCCCAACATCACCGTGA
ACGACACCATGTGCGCCCTGGTGGGCGGCGCCTTCCGCCGCTACTACCAGAGCCTGCACCTGCACCCCGA
GCAGATGCTGATGCGCGCCACCGTGCCCATCAACATCCGCCCCAGCACCACCGCCCCCATCAAGATGGAG
AACGACTTCACCATCGTGTTCAAGAGCCTGCCCATCCACCTGCCCACCCCCGAGGAGCGCATCGCCCACT
TCCACGTGCGCATGGGCTTCCTGAAGCGCGGCATCGAGCCCCTGCTGAGCATGTTCCTGCAGCACCTGCT
GACCTGGCTGCCCGAGCCCCTGATGCGCCTGATCGTGCTGCGCTTCACCATCTGCAGCAGCGCCGTGCTG
ACCAACGTGCTGAGCAGCACCGTGCCCTTCAGCCTGTGCGGCCAGCCCCTGACCACCGCCGCCTTCTGGG
TGCCCACCAGCGGCGACATCGGCATCGGCATCAGCATCATGACCTACTGCGACACCGTGGCCATCAACTT
CATCGCCGACGAGAACCTGATCGCCGACTGGGCCCCCGTGGTGCAGTTCATGCGCGAGGAGTGGGAGGAG
ATGAAGGGCATCCTGGGCAAGGAGCAGCACCTGCCCGTGATGGAGCCCCAGAAGACCGTGGAGCTGGTGA
ACCTGTGGCGCACCTGGGGCTTCCCCTGGAACACCCGC
SEQ ID NO. 11
Amino Acid
WSD3
Euglena gracilis
MVDSQPARPEGGARAVNRKLTKLGWSTLVTETSTNLSVPITIMVLETPITLPELYDILQERLLRQHSRYR
SLVQGTGELVELPIEDVVLEQHVRVHQLGDPDSQRELNTVLGNLSCLPLVMTRPLWEVVLIPKFKSGSVL
VFRNHHCLSDGGGGAIIVDSISDSPEQWEPKRKPALGEHILQLLALTLTLLASVPFVLYSVILVVLFPDR
PSPLKPKQLEGGRRKVAISGPISVPALKKVCRANNCKINDLALTLYAQALRDQAKAIDPTFDKPVWSGIP
VDVRLRGEVYTGNKFGFGVCRLPLHIAAFPEALAYVQKRMTFMKEHNLAMVMYYFSVVSSALMPTALLRA
MLAFNTRRISLVVSNVAAGNKQLVLKGHAIQYMYALVPPPPNVGIGCSVIGQQDQLVFGMVVDSAAAIDP
QAAIDHVLNALHVLSGGEI
SEQ ID NO. 12
DNA
WSD3-codon-optimized for expression in Chlamydomonas
Euglena gracilis
ATGGTGGACAGCCAGCCCGCCCGCCCCGAGGGCGGCGCCCGCGCCGTGAACCGCAAGCTGACCAAGCTGG
GCTGGAGCACCCTGGTGACCGAGACCAGCACCAACCTGAGCGTGCCCATCACCATCATGGTGCTGGAGAC
CCCCATCACCCTGCCCGAGCTGTACGACATCCTGCAGGAGCGCCTGCTGCGCCAGCACAGCCGCTACCGC
AGCCTGGTGCAGGGCACCGGCGAGCTGGTGGAGCTGCCCATCGAGGACGTGGTGCTGGAGCAGCACGTGC
GCGTGCACCAGCTGGGCGACCCCGACAGCCAGCGCGAGCTGAACACCGTGCTGGGCAACCTGAGCTGCCT
GCCCCTGGTGATGACCCGCCCCCTGTGGGAGGTGGTGCTGATCCCCAAGTTCAAGAGCGGCAGCGTGCTG
GTGTTCCGCAACCACCACTGCCTGAGCGACGGCGGCGGCGGCGCCATCATCGTGGACAGCATCAGCGACA
GCCCCGAGCAGTGGGAGCCCAAGCGCAAGCCCGCCCTGGGCGAGCACATCCTGCAGCTGCTGGCCCTGAC
CCTGACCCTGCTGGCCAGCGTGCCCTTCGTGCTGTACAGCGTGATCCTGGTGGTGCTGTTCCCCGACCGC
CCCAGCCCCCTGAAGCCCAAGCAGCTGGAGGGCGGCCGCCGCAAGGTGGCCATCAGCGGCCCCATCAGCG
TGCCCGCCCTGAAGAAGGTGTGCCGCGCCAACAACTGCAAGATCAACGACCTGGCCCTGACCCTGTACGC
CCAGGCCCTGCGCGACCAGGCCAAGGCCATCGACCCCACCTTCGACAAGCCCGTGTGGAGCGGCATCCCC
GTGGACGTGCGCCTGCGCGGCGAGGTGTACACCGGCAACAAGTTCGGCTTCGGCGTGTGCCGCCTGCCCC
TGCACATCGCCGCCTTCCCCGAGGCCCTGGCCTACGTGCAGAAGCGCATGACCTTCATGAAGGAGCACAA
CCTGGCCATGGTGATGTACTACTTCAGCGTGGTGAGCAGCGCCCTGATGCCCACCGCCCTGCTGCGCGCC
ATGCTGGCCTTCAACACCCGCCGCATCAGCCTGGTGGTGAGCAACGTGGCCGCCGGCAACAAGCAGCTGG
TGCTGAAGGGCCACGCCATCCAGTACATGTACGCCCTGGTGCCCCCCCCCCCCAACGTGGGCATCGGCTG
CAGCGTGATCGGCCAGCAGGACCAGCTGGTGTTCGGCATGGTGGTGGACAGCGCCGCCGCCATCGACCCC
CAGGCCGCCATCGACCACGTGCTGAACGCCCTGCACGTGCTGAGCGGCGGCGAGATCTAA
SEQ ID NO. 13
Amino Acid
WSD5
Euglena gracilis
MAVPGIKVSTKLTATDLFWWRVDEPQNPMVINILVEFEGVLTPAAVRDALEAAVAENIRLHGVPTSRFAD
TAGTWGLLAGCLTVLATGSQWYWKPIPHFSLEEHIRLHVLEERSEDCLRRFVDEEISHQLPKDRAQWRGI
VIHNTPGSGSRALFRFHHVIADGAGLGQWFYGLCQVHGPPTGDSPHEVPEKQAWVGRHPSTLSAHPPPKR
TAVQRLRKVAARVRDVVDFLLLEVLLVVYSALKLLFLSRDSNSPFKGPNTGRKKTGTTLHSLDLPVEAVK
ALGKGYDRDITVNDVLCTLLAGAFRRFFQRHLLHPEQMSMRVAVPINMRSSIRPPITMDNRFSLVFKSLP
IHLPTVQERLASFHVRMGLMKMSIEPRLGLLLMYFLAWMPERVLARVIEHFTLCTSAVLTNVMSSRIKLS
FAGQPMDNMCFWVPTSGDIGLGISVCTYCDRINLGLVVDENLLADVKPLLADVVAEWDDMQRQLSAQGAA
HPSSVIPAHTQEMIEANQQYGKPGHSR
SEQ ID NO. 14
DNA
WSD5-codon-optimized for expression in Chlamydomonas
Euglena gracilis
ATGGCCGTGCCCGGCATCAAGGTGAGCACCAAGCTGACCGCCACCGACCTGTTCTGGTGGCGCGTGGACG
AGCCCCAGAACCCCATGGTGATCAACATCCTGGTGGAGTTCGAGGGCGTGCTGACCCCCGCCGCCGTGCG
CGACGCCCTGGAGGCCGCCGTGGCCGAGAACATCCGCCTGCACGGCGTGCCCACCAGCCGCTTCGCCGAC
ACCGCCGGCACCTGGGGCCTGCTGGCCGGCTGCCTGACCGTGCTGGCCACCGGCAGCCAGTGGTACTGGA
AGCCCATCCCCCACTTCAGCCTGGAGGAGCACATCCGCCTGCACGTGCTGGAGGAGCGCAGCGAGGACTG
CCTGCGCCGCTTCGTGGACGAGGAGATCAGCCACCAGCTGCCCAAGGACCGCGCCCAGTGGCGCGGCATC
GTGATCCACAACACCCCCGGCAGCGGCAGCCGCGCCCTGTTCCGCTTCCACCACGTGATCGCCGACGGCG
CCGGCCTGGGCCAGTGGTTCTACGGCCTGTGCCAGGTGCACGGCCCCCCCACCGGCGACAGCCCCCACGA
GGTGCCCGAGAAGCAGGCCTGGGTGGGCCGCCACCCCAGCACCCTGAGCGCCCACCCCCCCCCCAAGCGC
ACCGCCGTGCAGCGCCTGCGCAAGGTGGCCGCCCGCGTGCGCGACGTGGTGGACTTCCTGCTGCTGGAGG
TGCTGCTGGTGGTGTACAGCGCCCTGAAGCTGCTGTTCCTGAGCCGCGACAGCAACAGCCCCTTCAAGGG
CCCCAACACCGGCCGCAAGAAGACCGGCACCACCCTGCACAGCCTGGACCTGCCCGTGGAGGCCGTGAAG
GCCCTGGGCAAGGGCTACGACCGCGACATCACCGTGAACGACGTGCTGTGCACCCTGCTGGCCGGCGCCT
TCCGCCGCTTCTTCCAGCGCCACCTGCTGCACCCCGAGCAGATGAGCATGCGCGTGGCCGTGCCCATCAA
CATGCGCAGCAGCATCCGCCCCCCCATCACCATGGACAACCGCTTCAGCCTGGTGTTCAAGAGCCTGCCC
ATCCACCTGCCCACCGTGCAGGAGCGCCTGGCCAGCTTCCACGTGCGCATGGGCCTGATGAAGATGAGCA
TCGAGCCCCGCCTGGGCCTGCTGCTGATGTACTTCCTGGCCTGGATGCCCGAGCGCGTGCTGGCCCGCGT
GATCGAGCACTTCACCCTGTGCACCAGCGCCGTGCTGACCAACGTGATGAGCAGCCGCATCAAGCTGAGC
TTCGCCGGCCAGCCCATGGACAACATGTGCTTCTGGGTGCCCACCAGCGGCGACATCGGCCTGGGCATCA
GCGTGTGCACCTACTGCGACCGCATCAACCTGGGCCTGGTGGTGGACGAGAACCTGCTGGCCGACGTGAA
GCCCCTGCTGGCCGACGTGGTGGCCGAGTGGGACGACATGCAGCGCCAGCTGAGCGCCCAGGGCGCCGCC
CACCCCAGCAGCGTGATCCCCGCCCACACCCAGGAGATGATCGAGGCCAACCAGCAGTACGGCAAGCCCG
GCCACAGCCGC
SEQ ID NO. 15
Amino Acid
fatty acid reductase 1
Arabidopsis thaliana
MESNCVQFLGNKTILITGAPGFLAKVLVEKILRLQPNVKKIYLLLRAPDEKSAMQRLRSEVMEIDLFKVL
RNNLGEDNLNALMREKIVPVPGDISIDNLGLKDTDLIQRMWSEIDIIINIAATTNFDERYDIGLGINTFG
ALNVLNFAKKCVKGQLLLHVSTAYISGEQPGLLLEKPFKMGETLSGDRELDINIEHDLMKQKLKELQDCS
DEEISQTMKDFGMARAKLHGWPNTYVFTKAMGEMLMGKYRENLPLVIIRPTMITSTIAEPFPGWIEGLKT
LDSVIVAYGKGRLKCFLADSNSVFDLIPADMVVNAMVAAATAHSGDTGIQAIYHVGSSCKNPVTFGQLHD
FTARYFAKRPLIGRNGSPIIVVKGTILSTMAQFSLYMTLRYKLPLQILRLINIVYPWSHGDNYSDLSRKI
KLAMRLVELYQPYLLFKGIFDDLNTERLRMKRKENIKELDGSFEFDPKSIDWDNYITNTHIPGLITHVLK
Q
SEQ ID NO. 16
Amino Acid
Wax Synthase -O-acyltransferase WSD1
Arabidopsis thaliana
MKAEKVMEREIETTPIEPLSPMSHMLSSPNFFIVITFGFKTRCNRSAFVDGINNTLINAPRFSSKMEINY
KKKGEPVWIPVKLRVDDHIIVPDLEYSNIQNPDQFVEDYTSNIANIPMDMSKPLWEFHLLNMKTSKAESL
AIVKIHHSIGDGMSLMSLLLACSRKISDPDALVSNTTATKKPADSMAWWLFVGFWFMIRVTFTTIVEFSK
LMLTVCFLEDTKNPLMGNPSDGFQSWKVVHRIISFEDVKLIKDTMNMKVNDVLLGMTQAGLSRYLSSKYD
GSTAEKKKILEKLRVRGAVAINLRPATKIEDLADMMAKGSKCRWGNFIGTVIFPLWVKSEKDPLEYIRRA
KATMDRKKISLEAFFFYGIIKFTLKFFGGKAVEAFGKRIFGHTSLAFSNVKGPDEEISFFHHPISYIAGS
ALVGAQALNIHFISYVDKIVINLAVDTTTIQDPNRLCDDMVEALEIIKSATQGEIFHKTE
SEQ ID NO. 17
Amino Acid
DGAT2
Arabidopsis thaliana
MGGSREFRAEEHSNQFHSIIAMAIWLGAIHFNVALVLCSLIFLPPSLSLMVLGLLSLFIFIPIDHRSKYG
RKLARYICKHACNYFPVSLYVEDYEAFQPNRAYVFGYEPHSVLPIGVVALCDLTGFMPIPNIKVLASSAI
FYTPFLRHIWTWLGLTAASRKNFTSLLDSGYSCVLVPGGVQETFHMQHDAENVFLSRRRGFVRIAMEQGS
PLVPVFCFGQARVYKWWKPDCDLYLKLSRAIRFTPICFWGVFGSPLPCRQPMHVVVGKPIEVTKTLKPTD
EEIAKFHGQYVEALRDLFERHKSRVGYDLELKIL
SEQ ID NO. 18
Amino Acid
beta-ketoacyl-CoA synthase (KCS)
Arabidopsis thaliana
MSHNQNQPHRPVPVHVTNAEPNPNPNNLPNFLLSVRLKYVKLGYHYLISNALYILLLPLLAATIANLSSF
TINDLSLLYNTLRFHFLSATLATALLISLSTAYFTTRPRRVFLLDFSCYKPDPSLICTRETFMDRSQRVG
IFTEDNLAFQQKILERSGLGQKTYFPEALLRVPPNPCMEEARKEAETVMFGAIDAVLEKTGVKPKDIGIL
VVNCSLFNPTPSLSAMIVNKYKLRGNILSYNLGGMGCSAGLISIDLAKQMLQVQPNSYALVVSTENITLN
WYLGNDRSMLLSNCIFRMGGAAVLLSNRSSDRSRSKYQLIHTVRTHKGADDNAFGCVYQREDNNAEETGK
IGVSLSKNLMAIAGEALKTNITTLGPLVLPMSEQLLFFATLVARKVFKVKKIKPYIPDFKLAFEHFCIHA
GGRAVLDEIEKNLDLSEWHMEPSRMTLNRFGNTSSSSLWYELAYSEAKGRIKRGDRTWQIAFGSGFKCNS
AVWKALRTIDPMDEKTNPWIDEIDDFPVQVPRITPITSS
SEQ ID NO. 19
Amino Acid
β-ketoacyl-CoA reductase (KCR)
Arabidopsis thaliana
MEICTYFKSQPTWLLILFVLGSISIFKFIFTLLRSFYIYFLRPSKNLRRYGSWAIITGPTDGIGKAFAFQ
LAQKGLNLILVARNPDKLKDVSDSIRSKYSQTQILTVVMDFSGDIDEGVKRIKESIEGLDVGILINNAGM
SYPYAKYFHEVDEELINNLIKINVEGTTKVTQAVLPNMLKRKKGAIINMGSGAAALIPSYPFYSVYAGAK
TYVDQFTKCLHVEYKKSGIDVQCQVPLYVATKMTKIRRASFLVASPEGYAKAALRFVGYEAQCTPYWPHA
LMGAVVSALPESVFESFNIKRCLQIRKKGLQKDSMKKE
SEQ ID NO. 20
Amino Acid
β-hydroxyacyl-CoA dehydratase (HCD)
Arabidopsis thaliana
MAGFLSVVRRVYLTLYNWIVFAGWAQVLYLAITTLKETGYENVYDAIEKPLQLAQTAAVLEILHGLVGLV
RSPVSATLPQIGSRLFLTWGILYSFPEVRSHFLVTSLVISWSITEIIRYSFFGFKEALGFAPSWHLWLRY
SSFLLLYPTGITSEVGLIYLALPHIKTSEMYSVRMPNILNFSFDFFYATILVLAIYVPGSPHMYRYMLGQ
RKRALSKSKRE
SEQ ID NO. 21
Amino Acid
enoyl-CoA reductase (ECR)
Arabidopsis thaliana
MKVTVVSRSGREVLKAPLDLPDSATVADLQEAFHKRAKKFYPSRQRLTLPVTPGSKDKPVVLNSKKSLKE
YCDGNNNSLTVVFKDLGAQVSYRTLFFFEYLGPLLIYPVFYYFPVYKFLGYGEDCVIHPVQTYAMYYWCF
HYFKRILETFFVHRFSHATSP1GNVFRNCAYYWSFGAYIAYYVNHPLYTPVSDLQMKIGFGFGLVCQVAN
FYCHILLKNLRDPSGAGGYQIPRGFLFNIVTCANYTTEIYQWLGFNIATQTIAGYVFLAVAALIMTNWAL
GKHSRLRKIFDGKDGKPKYPRRWVILPPFL
SEQ ID NO. 22
DNA
NIT1 promoter
Chlamydomonas reinhardtii
TCGAGGGTGCCCCGCCAGCCCCCGCTCCTCTGCTGCCTCTGATGCCTCATGCCAAAAGTCCTGACGCGGC
GCCCTCACATCCCCGTCCGGGTAATCTATGAGTTTCCCTTATCGAGCATGTACGCGATAGTGGACGGGGC
TCAGGGTGGGGGGTGGGTGGGTGGGAGGGGCGTTCCTTCAGACACCCTGGAGGGGTGGCTAGAAAAGCGG
CCGCGCGCCAGAAATGTCTCGCTGCCCTGTGCAATAAGCACCGGCTATATTGCTCAGCGCTGTTCGGCGC
AACGGGGGGTCAGCCCTTGGGAAGCGTTGGACTATATGGTAGGGTGCGAGTGACCCCGCGCGACTTGGAG
CTCGATGGCCCCGGGTTGTTTGGGGCGTCCGCCTCTCGCGCTATTCTGAGCTGGAGACCGAGGCGCATGA
AAATGCATTCGCTTCCATAGGACGCTGCATTGTGGCTTGAAGGTTCAAGGGAAGGGTTCAAACGACCCCG
CCGTACGAACTTTTGTCGGGGGGCGCTCCCGGCCCCGGGCTCTTGTGCGCGCATTAGGGCTTCGGGTCGC
AAGCAAGACGATACAGGAACCGACCAATCGATAGTCTTGTGCGACCGTGCACGTGTGCAGCAATAGTTAG
GTCGATAACCACGTTGAACTTGCGTCTCTCTTCGTGGCGCCTCCTGCTTGGTGCTCCACTTCACTTGTCG
CTATATAGCACAGCGTTGAAAGCAAAGGCCACACTAATACAGCCGGGCTCGAGAGTCCGTCTGCGTTTGC
ATTGTTGGCCAAGGGCTGCTTTGTAGCCAAAGCCATACACGAAGCTTCACTTGATTAGCTTTACGACCCT
CAGCCGAATCCTGCCAGTGAATTC
SEQ ID NO. 23
DNA
CYC6 promoter
Chlamydomonas reinhardtii
CTCGAGCTCGAGCAGAGGTTGGGAATCGCTTTGAAAATCCAGCAATCGGGTCTCAGCTGTCTCAGGCCGC
ACGCGCCTTGGACAAGGCACTTCAGTAACGTACTCCAAGCCCTCTATCTGCATGCCCACAAAGCGCAGGA
ATGCCGACCATCGTGCCAGACTGTGCCGCGCCCGAACCGAAATCCGTCACTCCCCTTGGTTCCCATGGTG
GCATGGTCCCCCCTGTTCGCCCAAAGCCTGGTTCAGCGCCCAGTGGCAAACGGCTTTGGCTCAGCTCCTT
GGTATTGCTGGTTTCTAGCAATCTCGTCCGTTCCTCTGTTGCCAATGTAGCAGGTGCAAACAGTCGAATA
CGGTTTTACTCAGGGGCAATCTCAACTAACAGAGGCCCTGGGCCTGTTGCCTGGAACCTATGAAGACGAT
AATGCCACGGCGACTTTCGAGCCTGAGGGAAGTTTGCACCGGTACCGCATTGTGCAAGGTTACGGTACAT
GATAGGGGGAGTGCGACGCGGTAAGGCTTGGCGCAGCTTGGCGCGTCTGCCTTGCATGCATGTCCGAAAC
ACGCCACGTCGCGCCACGAAAAGCGGTAAAAGGACCTGCCATGGTCCTCCAGGGTGTTACCACTTCCATT
TCGCTCAGCTGGGATGGTGCTCGTAGGTGCACCAGCGTTGATTATTTCAGGCAGGAAGCGGCTGCGAAGC
CCGCCTTTCACTGAAGACTGGGATGAGCGCACCTGTACCTGCCAGTATGGTACCGGCGCGCTACCGATGC
GTGTAGTAGAGCTTGCTGCCATACAGTAACTCTGGTACCCCCAGCCACCGGGCGTAGCGAGCAGACTCAA
TAAGTATGATGGGTTCTTATTGCAGCCGCTGTTACAGTTTACAGCGCAAGGGAACACGCCCCTCATTCAC
AGAACTAACTCAACCTACTCCATCGACGAATTC
SEQ ID NO. 24
Amino Acid
fII
Synechococcus elongatus PCC 7942
MEKTIGLEIIEVVEQAAIASARLMGKGEKNEADRVAVEAMRVRMNQVEMLGRIVIGEGERDEAPMLYIGE
EVGIYRDADKRAGVPAGKLVEIDIAVDPCEGTNLCAYGQPGSMAVLAISEKGGLFAAPDFYMKKLAAPPA
AKGKVDINKSATENLKILSECLDRAIDELVVVVMDRPRHKELIQEIRQAGARVRLISDGDVSAAISCGFA
GTNTHALMGIGAAPEGVISAAAMRCLGGHFQGQLIYDPEVVKTGLIGESRESNIARLQEMGITDPDRVYD
ANELASGQEVLFAACGITPGLLMEGVRFFKGGARTQSLVISSQSRTARFVDTVHMFDDVKTVSLR
SEQ ID NO. 25
DNA
fII
Synechococcus elongatus PCC 7942
ATGGAGAAGACCATCGGCCTGGAGATCATCGAGGTGGTGGAGCAGGCCGCCATCGCCAGCGCCCGCCTGA
TGGGCAAGGGCGAGAAGAACGAGGCCGACCGCGTGGCCGTGGAGGCCATGCGCGTGCGCATGAACCAGGT
GGAGATGCTGGGCCGCATCGTGATCGGCGAGGGCGAGCGCGACGAGGCCCCCATGCTGTACATCGGCGAG
GAGGTGGGCATCTACCGCGACGCCGACAAGCGCGCCGGCGTGCCCGCCGGCAAGCTGGTGGAGATCGACA
TCGCCGTGGACCCCTGCGAGGGCACCAACCTGTGCGCCTACGGCCAGCCCGGCAGCATGGCCGTGCTGGC
CATCAGCGAGAAGGGCGGCCTGTTCGCCGCCCCCGACTTCTACATGAAGAAGCTGGCCGCCCCCCCCGCC
GCCAAGGGCAAGGTGGACATCAACAAGAGCGCCACCGAGAACCTGAAGATCCTGAGCGAGTGCCTGGACC
GCGCCATCGACGAGCTGGTGGTGGTGGTGATGGACCGCCCCCGCCACAAGGAGCTGATCCAGGAGATCCG
CCAGGCCGGCGCCCGCGTGCGCCTGATCAGCGACGGCGACGTGAGCGCCGCCATCAGCTGCGGCTTCGCC
GGCACCAACACCCACGCCCTGATGGGCATCGGCGCCGCCCCCGAGGGCGTGATCAGCGCCGCCGCCATGC
GCTGCCTGGGCGGCCACTTCCAGGGCCAGCTGATCTACGACCCCGAGGTGGTGAAGACCGGCCTGATCGG
CGAGAGCCGCGAGAGCAACATCGCCCGCCTGCAGGAGATGGGCATCACCGACCCCGACCGCGTGTACGAC
GCCAACGAGCTGGCCAGCGGCCAGGAGGTGCTGTTCGCCGCCTGCGGCATCACCCCCGGCCTGCTGATGG
AGGGCGTGCGCTTCTTCAAGGGCGGCGCCCGCACCCAGAGCCTGGTGATCAGCAGCCAGAGCCGCACCGC
CCGCTTCGTGGACACCGTGCACATGTTCGACGACGTGAAGACCGTGAGCCTGCGC
SEQ ID NO. 26
Amino Acid
fatty acyl-CoA reductase 1
Apis mellifera
MSTISDNQCTSVRDFYKDRSIFITGGTGFMGKVLVEKLLRSCPGIKNIYILMRPKKSQDIQQRLQKLLDV
PLFDKLRRDTPDELLKIIPIAGDVTEHELGISEADQNVIIRDVSIVFHSAATVKFDEPLKRSVHINMIGT
KQLLNLCHRMHNLEALIHVSTAYCNCDRYDVAEEIYPVSAEPEEIMALTKLMDSQMIDNITPTLIGNRPN
TYTFTKALTERMLQSECGHLPIAIVRPSIVLSSFREPVSGWVDNLNGPTGIVAAAGKGFFRSMLCQKNMV
ADLVPVDIVINLMICTAWRTATNRTKTIPIYHCCTGQQNPITWQQFVELILKYNRMHPPNDTIWWPDGKC
HTFAIVNNVCKLFQHLLPAHILDFIFRLRGKPAIMVGLHEKIDKAVKCLEYFTMQQWNFRDDNVRQLSGE
LSPEDRQIFMFDVKQIDWPSYLEQYILGIRQFIIKDSPETLPAARSHIKKLYWIQKVVEFGMLLVVLRFL
LLRIPMAQSACFTLLSAILRMCRMIV
SEQ ID NO. 27
Amino Acid
Fatty acyl-CoA reductase
Apis cerana cerana
MDKIKIVQSNNKENLKNTSDSQIQKFYTGKYIFFTGCTSILGSSILEKILISCTEISKIYIMIKLKNDIL
IKEQLKKYFQNEIFNTVRESNPNFMEKVVPIYGDLSKADLGLSSEDRRCLIENVNIIIHNGSIVQSTKVS
YILRLNVIATQTLLELAMECSHLEAFVYVSTAFSHPYKQIIEEKFYPIYAGNIKIIEDVIRADEENESGI
TNEALRDIITDWVNLYIFSKAYAEDLVYNFGKKKSLPCVVFRPSMVVCTNEKLVPSKNKNGPVMLATAIS
LGYIHVSNLKKTDTMDLIPIDMTVNSLLAMIWDFVVYRKKEEPQQVYNYGSTDWNPITVDSASKMIFKEI
EKNPSDNVIWKPYLIYIQNIYLFSILNILLNVIPNILIDLILLISKGEQPPIMRTIHKLKKHYFPFIQIF
RSNQIIKTNKFKECLTRMNTTDLKEFSFNLATLNWNDSVVKLMTCCRKEMNEP1TASPATKKKYQNLIEG
KGLQNSTTPLLYIE
SEQ ID NO. 28
Amino Acid
fatty acyl-CoA reductase 1-like
Apis dorsata
MDKIKIVQSDKENLKNTSDSQIQKFYTGKHIFFTGCTSFLGSSILEKILITCTEISKIYVMIKLKNDVLI
KEQLKKYFQNEIFDTLRESNPNFIEKVVPIYGDLSKADLGLSSKNRRCLIENVNIIIHNGSIIQSPKASY
ILRLNVIATQTLLELATECSHLEAFVYVSTAFSHPYKQIIEEKFYPIAGNIKIIEDVIRADEENESGITN
EALRNIMGDWVNLYAFSKAYAEDLVYNFGKTKSLPCVVFRPSMVVCTNEKLVPSKNKNGPVMLAMAISLG
YIHVSNLKKTDTMDLIPIDMTANSLLAMIWDFVVYRKKEELQQVYNYGSTDWNPITVGSASEIIFKEVEK
NPSNNVLWKPYLIYIQNIYLFSTLNILLNVIPGILIDLTLLICQEEPPIMRTIHKLKKHYLPFIQIFRPN
QIIKTNKFKECLTRMNTTDLKEFSFNLATMNWNDNAVKLMTCCRKEMNEPTTASPATKKKYRNLVKLHFV
ICSLLIMLFLLYFFYRILSIFCHCYHH
SEQ ID NO. 29
Amino Acid
fatty acyl-CoA reductase 1
Anas platyrhynchos
MVSIPEYYEGKNVLLTGATGFMGKVLLEKLLRSCPKVQAVYVLVRHKSGQTPEARIQEITSCKLFDRLRE
EQPDFKEKIIVITSELTQPELDLSSPIKQKLIDCINIIFHCAATVRFNETLRDAVQLNVLSTKQLLSLAH
QMTNLEVFIHVSTAYAYCNRKHIEEIVYPPPVDPKKLMDSLEWMDDGLVNDITPKLIGDRPNTYTYTKAL
AEYVVQQEGAKLNTAIIRPSIVGASWKEPFPGWIDNFNGPSGLFIAAGKGILRTMRATNGAVADLVPVDV
VVNMTLAAAWYSGVNRPRNIMVYNCTTGGTNPFHWSEVEYHVISTFKRNPLEQAFRRPNVNLTSNHLLYH
YWIAVSHKAPAFLYDIYLRITGRSPRMMKTISRLHKAMMLLEYFTSNSWIWNTENMTMLMNQLTPEDKKT
FNFDVRQLHWAEYMENYCMGTKKYVLNEEMSGLPAARKHLNKLRNIRYGFNTILVILIWRIFIARSQMAR
NIWYFVVSLCYKFLSYFRASSTMRY
SEQ ID NO. 30
Amino Acid
fatty acyl-CoA reductase 2
Canis lupus familiaris
MSMIAAFYSGKSILITGATGFMGKVLMEKLFRTSPDLKVIYILVRPKAGQTTQQRVFQILNSKLFEKVKE
VCPNVHEKIRAIYADLNQNDFAISKEDMQELLSCTNIVFHCAATVRFDDHLRHAVQLNVTATQQLLLMAS
QMPKLEAFIHISTAFSNCNLKHIDEVIYPCPVEPKKIIDSMEWLDDAIIDEITPKLIGDRPNTYTYTKAL
GEMVVQQESGNLNIAIIRPSIVGATWQEPFPGWVDNLNGPSGLIIAAGKGFLRAIRATPMAVADLIPVDT
VVNLTLAVGWYTAVHRPKSTLIYHCTSGNLNPCNWGKMGFQVLATFEKIPFERAFRRPYADFTTNTITTQ
YWNAVSHRAPAIIYDFYLRLTGRKPRMTKVMNRLLRTVSMLEYFVNRSWEWSTYNTEMLMSELSPEDQRV
FNFDVRQLNWLEYIENYVLGVKKYLLKEDMAGIPEAKQHLKRLRNIHYLFNTALFLIAWRLLIARSQMAR
NVWFFIVSFCYKFLSYFRASSTLKV
SEQ ID NO. 31
DNA
long-chain-alcohol O-fatty-acyltransferase
Beta vulgaris subsp. vulgaris
TGTGTAATTTCTCTACCAGGGGCTAATAGCCTAATCTATCAAAAAGATTTAAGAATGCCCGATCTGAATC
CGACATGATTTTTGTTTGTCGGGAAATACTATCAAATTAAAGCTTGCTGAGCAAAATGGAAATTGATCAC
TCCTAATTACTATTGGTTTTTTTACCGAAATGAAACAAAGAATAGAGATATTCCTAGCAACTAGCATAAA
AGGTCAACCGTGAATCTTGGATTTGTTTCTGCATCATATAAAGCCTTGCGAGTATCTGCTTGTATATACT
AGCAATTAGGCAATTAACTGAGCACACAAACACAATCGAGCAGATAGATCAGCAAATAGGAAAAGAATGG
AGTCTGAGATTAAGAATTTCATGAAGATCTGGTTATTCGCAATTTGTTCAGCTTGTTACTCCCTGAGTTT
ATCCAGAATATTCCACATCCGAAGCGGCATTCCAAGGTTACTCTTCATCCTCCCCATCATCTATCTCTTT
ACTGTTCTCCCTTTATCTCTCTCTTCTTTTCATCTTGGTGGTCCCACTATCTTCTTCCTTGTTTGGCTTG
CTAATTTTAAACTTCTTCTTTACGCCTTTGATCTTGGTCCTCTTTCTACTAATCCAATTACAAACAACAA
CAACAACAACAACAACAACGTTAATTCCCTATCTCTCTCTCATTTCATTTCCATTGCTCTTCTTCCCATT
AAAGTCAATCAACAACAACCATCAAAACCCACAAATAATAAGTGGAAGTCTGTTCTCATCATTGCCTTCA
AATTACTGGCATTTGCTCTTGTCATCAAAATCTATGACTTTACCCAACATTTACCCAAATTTCTTCTATT
GATTAATTACTGCTGTCATCTTTACCTTGGTGTTGAGGTAACTTTAGCTGTTGTTGCAGCCATAGTTCGG
GCCACTTTGGGCTTGGGCCTTGACCCACAGTTTAATGAGCCTTATTTGGCCACATCACTTCAGGATTTTT
GGGGCCGTAGATGGAATCTGATGGTGTCAGACATCCTACGCCTCTCCGTTTTTAACCCCATCCGACGTGT
CTTCTCTCCATTGGTTGGCAAGAGGTGGGCCCTGGTAGTTGGAATGATTGCGGCATTTACTGTGTCTGGC
CTCATGCACGAGCTCATCTTCTATTATTTCACACGTGTGAACCCCACGTGGGAAGTCACGTGGTTTTTTG
TATTACATGGGATGTGTACGGCGGTTGAAGTGGTGGTTAAGGAGGCAGTTGGTGGTCGGTTGCAGTTGCA
TCGGTTGATTTCGGGGACGTTGACGATTGGGTTTGTTGCAGTTACGGCGTGGTGGCTTTTTTTACCTCAA
ATCATAAGAAATGGTGTGGATGTCAAAGTCATTAATGAGTATCCTGTAATGTTTAGCTTTGTCAAACAAC
ACATTTTTTTTTGTTTCAAAAACTGATTCAATTTTCGATTGTTTCTCACTACAATAGCATGCTCAGTCTT
GGAATGCTTTCAGTACAATAGTTCAGTTTTTTTATTATCTAAGTAGTTTCTTTTATGATGTAATTTTTCA
TCTTAATCATAATTCAACTTGGTTGCTTCATTTCAA
SEQ ID NO. 32
DNA
long-chain-alcohol O-fatty-acyltransferase-like
Spinacia oleracea
TTAATGGCGAGTGAATTCAAGAAGAATCAGCAACAGATTTCTTCGAGAGGCTAAATGGCGGAACATGGAG
GAGGACCACCGCTTCACCGGCATCATTTGGAGGTGGTTGCCATTACAGAACTGCTATAATTTGAACCGTT
GGATTCTGTTGTAGTGGTGGACCTGGTGACTGGTAAACCAATTTAATAATACTGTATAAAATCTGTTATT
CCATTACCACCAACAAAAACTCACAAAAAATAACCCTAAAAACCAAAAATGGAGACAGAGATCTGGAATT
TCATCAAGATATGGGGAATAGCAATCGCTTCAGCCTGCTACTCCTACTCTTTATCCAGAACCTTCCACAT
CCAAACCGGTATTCTCCGGTTGTTCTTCATCCTTCCCGTCATCTACCTCTTCACTGTCCTCCCACTTTCT
CTCTCCTCCTTCCATCTCGGTGGTCCCACCATCTTCTACCTTGTTTGGCTTGCCAACTTTAAACTACTCC
TCTACTCCTTCAACCTCGGCCCTCTTTCTTCCAATCCAAACACCTCCTTATCGCATTTCATCGCCATTGC
TCTTCTCCCCATCAAAGTCAACGCCGGTTCAACGCCGACCAAGAAGCGGGACCCACTCGGATCACTTCCT
CTGTTTGTTGTAAAATTACTGGCTTTTGCCCTTGTGGTAAAAGTTTATGAGTTTCGCCAAGATTTACCTA
AATCCCTTCTTTTGCTTAATTACTGTTGTCATCTTTACCTTGGTGTAGAGGTTACTTTGGGAATTACCGC
GGCCTTGGTTCGGGCCAGTTTGGGGTTGGGCTTGGACCCACAGTTTGATGAGCCGTACTTGGCCACCTCA
CTCCAGGACTTTTGGGGCCGTAGATGGAATCTCATGGTGTCGGACATCTTACGGTTGTCCGTTTACGACC
CCATCCGACGTGTTGTCTCGCCATTGGTTGGGAAAAGGTGCGGTTTAGTGGGTGGGATCGTAATGTCGTT
TACTGTGTCTGGGCTGATGCACGAGGTGATTTTCTATTATTTCACACGTGTGAGGCCCACGTGGGAAGTC
ACGTGGTTCTTTGTTCTACATGGGGTGTGCACCGCAGTGGAGGTGGTGGTTAAGAAAATGGTTGTATCGA
GGTTTCAGTTGCATCGATTGATATCAGGTCCGTTGACAATTGGATTCATAGGGGTCACAGCATGGTGGTT
ATTCCTCCCTCAAATCTTAAGGAATGGTTTCGACGTCAAAGTTATAAATGAGTATCCTGTAATGGTTAAT
TTTGTTAAGGAAAATGTTTTGTACTTTTTTATTTTGTTGGACAAACTTTTGGTGGCTTGAGCAATTTTGA
TTTCGTCTATGTAGTCAACTCGGTTATGATTTATGAATGTTATTTTTCAACTTAA
SEQ ID NO. 33
DNA
acyl-CoA--sterol O-acyltransferase 1-like
Coffea Arabica
AAATTGGAATAGTTTCGTACGCTCCTTTGCTCATTCCAGTCCGCCCGATAAAGCAAGCTCTCTCATCCCA
CCGACGCCACAGAAGCCTTGTTTAACAAGTGGTCGCCGTCCGGGGGAATTCGCAAAACCATTTCCAAATG
GGGGACGAGATCAAGAGTTCAATCTTTGCTTTCGCATCGGTGCCAGCATCTCTCAGTTACTGCTATTTCA
TTGCCGCAAGAATCCCAAAAGGGTTCTTGAGGCTGATTTTTCTCCTACCCGTCTACTATCACTTCACAAT
TCTCCCTCTTTACATGCCCATTATCTTCTTTAGAGGTGTCTCAACACTCTTCATAACATGGCTCGGCAAC
TTCAAGCTGCTCCTCTTTGCCTTTGGACGAGGTCCACTCTCCTCGGACCAATCCATGCCCTTGCACATCT
TCATCGCCTCCTGTGCTCTCCCCATCAGAACCAAGCTGCCAAATGTCAACCCCTCATCTACTTCTTCCCG
ACCCTCCAAGAAAAAGCCATGGTTTTTAAATTTAGGAACGGAGATCTTAGCTTTATTCTCTTTATTTGGG
CTGGCAGCCAAATATGAAGAAACTGTACACCCCGTAGTTGTACAAATAGCCTATAGTTGCGCGATGTTTT
TCCTAATTGAAGTTCTGGTGACGCTCTCTAGTTCCGCGGTCCGAGCCCTGGTGGGTCTAGAGCTGGAGGC
ACCGTCCGACGAGCCCTACTTATCAGCTTCTCTGCAAGATTTCTGGGGCAAGAGGTGGAACCTCTCAGTA
ACAAATGCACTGCGGCACACAATATACAAGCCCGTCAGGTCAATATCGGCGGTCGTACTGGGGAATCGAT
CAGCCGCACTGCCTGCCATCTTCGCGACCTTTCTTGTCTCTGGTCTAATGCATGAACTCATATACTATTA
CCTCTCAGGTGTGAAGCCCTCCTGGGAAGTGACGTGGTTCTTCGTTCTGCATGGAATTTGTGTTGTGATT
GAAATGGTGTTGAAGACAGCTTTGGGAGGAAAATGGGCGGTGCCCCGGTTAATTGCGGCCCCGTTGACAC
TTGGGTTTGTGATTTCAACCGGTATGTGGTTGTTTTTCCCTCCGTTGACCGAGATGGGGATTGATAAAAT
GGTTTTTGAAGAGTTCAGTTGCGCTGGCGAGTATGTGAAGGGTAGGCTGGTGGCCTTATGTCCCACTATC
CTGGGCCACAAATCGAGGAGTTAAGACTCAGTCGGGCTCGGGCAGTCTGAAAACGACGACGGGCCATCAA
GAAATGTCTCCCACATTTCCGTCCTAATAAAATGGACAACTGTTTGTCCGTAGTTGACTTTAAAGTTCAA
TTATGCATGCGTGTGGTCCCCTTCTAGCGTTCAATTTCGGGATTATATATCTCATCTCAGTTGTAATATT
ATTGTCGCTTCCTCGTCACAATCAGAGACTGGATGCTGCGACTTTTCGCGTGCTTTCTGCAATTCAAGAG
CCGGTTTGGTTTTGGGTTGTTATCAAAATATATTAGTA
SEQ ID NO. 34
DNA
acyl-CoA--sterol O-acyltransferase 1-like
Cuscuta australis isolate Yunnan
ATGGAGAAGATCTCACTAACCCACGTCTGGTTTCTGGTTTTGGCTTCTCTGGTGTACTGCTATTTCGTGT
CTGCAAACCTCCCAAAGGGCATTTTCAGGTTCATATCTCTAACCCCTGTTTTCGGCCTCTTCGCTGTCTT
CCCTCTCCTCCACTCCTCCGCCTTCTGCACGGCGGTCGCCTTCTTCTTCTTCACCTGGCTCTCCAACTTC
AAGCTCCTCGCCTTCTCCTTCGACCGCGGCCCGCTCTCCTCCTCCTCACCCGCCTACAGGTCTCTCCTCA
CCTTCATTGCCATGGCTTCTCTTCCTCTCAGGTTGAAGAAGAAAAATGTCAATAGATCAAAGGTACAGAT
TTTGCGGTTAAACTTGGCGGCGGAAATTGCGGGCTTCGCGGGGTTGTTGCAGCTGATTTTCCGGTACGGA
GATGGGGCCCACCAGAACCTGGTCTTGATCTGGTATTCTCTCCTGGTTTTCCTCATGGTGGATGTGCTGG
TCGGAGTTTCGGGATTCGCGGTCCGGGTCTTGACCGGTCTAGATCTGGACCCGCCGTCGGACGAGCCTTA
CCTCTCCTGCTCCCTCCGGGAATTCTGGGGGAGGCGCTGGAACCTCACCGTGACCAACACCTTCCGCTTC
TCCGTCTACGATCCCGTCCGGGAACTCTCCGCCGCCGTCATCGGCGGCGCGTGGGCCCCACTTCCGGCGA
TGATGGCGACGTTCGCGCTCTCCGGCCTCATGCACGAGCTGCTGGTCTTCTACGTCGCGCGCGCCCGCCC
GTCGTGGGAGATGACGGCGTTCTTCTTGCTCCACGGAGTCTGCGTCGCGGCGGAGTACGCGACGGAGCAG
GCTTGGGGAGGCACTCCCCGGCTGCCGCGGGCGGTTTCGGGGCCGTTGACGGTCGGGTTCGTGGTGGGCA
CCACCTTCTGGCTGTTCTTCCCGCCGCTAATTAGGAGCGGCGCCGACAAAATGGTCCTGGAAGAATTGAA
ACCTATATCCAGTTCATTCATAACCATTGGAGATCATTAGTGATTGCGAAT
SEQ ID NO. 35
Amino Acid
long-chain-alcohol O-fatty-acyltransferase
Beta vulgaris subsp. vulgaris
MESEIKNFMKIWLFAICSACYSLSLSRIFHIRSGIPRLLFILPIIYLFTVLPLSLSSFHLGGPTIFFLVW
LANFKLLLYAFDLGPLSTNPITNNNNNNNNNVNSLSLSHFISIALLPIKVNQQQPSKPTNNKWKSVLIIA
FKLLAFALVIKIYDFTQHLPKFLLLINYCCHLYLGVEVTLAVVAAIVRATLGLGLDPQFNEPYLATSLQD
FWGRRWNLMVSDILRLSVFNPIRRVFSPLVGKRWALVVGMIAAFTVSGLMHELIFYYFTRVNPTWEVTWF
FVLHGMCTAVEVVVKEAVGGRLQLHRLISGTLTIGFVAVTAWWLFLPQIIRNGVDVKVINEYPVMFSFVK
QHIFFCFKN
SEQ ID NO. 36
Amino Acid
long-chain-alcohol O-fatty-acyltransferase-like
Spinacia Oleracea
METEIWNFIKIWGIAIASACYSYSLSRTFHIQTGILRLFFILPVIYLFTVLPLSLSSFHLGGPTIFYLVW
LANFKLLLYSFNLGPLSSNPNTSLSHFIAIALLPIKVNAGSTPTKKRDPLGSLPLFVVKLLAFALVVKVY
EFRQDLPKSLLLLNYCCHLYLGVEVTLGITAALVRASLGLGLDPQFDEPYLATSLQDFWGRRWNLMVSDI
LRLSVYDPIRRVVSPLVGKRCGLVGGIVMSFTVSGLMHEVIFYYFTRVRPTWEVTWFFVLHGVCTAVEVV
VKKMVVSRFQLHRLISGPLTIGFIGVTAWWLFLPQILRNGFDVKVINEYPVMVNFVKENVLYFFILLDKL
LVA
SEQ ID NO. 37
Amino Acid
acyl-CoA--sterol O-acyltransferase 1-like
Coffea Arabica
MGDEIKSSIFAFASVPASLSYCYFIAARIPKGFLRLIFLLPVYYHFTILPLYMPIIFFRGVSTLFITWLG
NFKLLLFAFGRGPLSSDQSMPLHIFIASCALPIRTKLPNVNPSSTSSRPSKKKPWFLNLGTEILALFSLF
GLAAKYEETVHPVVVQIAYSCAMFFLIEVLVTLSSSAVRALVGLELEAPSDEPYLSASLQDFWGKRWNLS
VTNALRHTIYKPVRSISAVVLGNRSAALPAIFATFLVSGLMHELIYYYLSGVKPSWEVTWFFVLHGICVV
IEMVLKTALGGKWAVPRLIAAPLTLGFVISTGMWLFFPPLTEMGIDKMVFEEFSCAGEYVKGRLVALCPT
ILGHKSRS
SEQ ID NO. 38
Amino Acid
acyl-CoA--sterol O-acyltransferase 1-like
Cuscuta australis
MEKISLTHVWFLVLASLVYCYFVSANLPKGIFRFISLTPVFGLFAVFPLLHSSAFCTAVAFFFFTWLSNF
KLLAFSFDRGPLSSSSPAYRSLLTFIAMASLPLRLKKKNVNRSKILRLNLAAEIAGFAGLLQLIFRYGDG
AHQNLVLIWYSLLVFLMVDVLVGVSGFAVRVLTGLDLDPPSDEPYLSCSLREFWGRRWNLTVTNTFRFSV
YDPVRELSAAVIGGAWAPLPAMMATFALSGLMHELLVFYVARARPSWEMTAFFLLHGVCVAAEYATEQAW
GGTPRLPRAVSGPLTVGFVVGTTFWLFFPPLIRSGADKMVLEELKTYIQFIHNHWRSLVIAN
SEQ ID NO. 39
Amino Acid
Pyruvate dehydrogenase E1 component subunit alpha
Homo Saipan
MRKMLAAVSRVLSGASQKPASRVLVASRNFANDATFEIKKCDLHRLEEGPPVTTVLTREDGLKYYRMMQT
VRRMELKADQLYKQKIIRGFCHLCDGQEACCVGLEAGINPTDHLITAYRAHGFTFTRGLSVREILAELTG
RKGGCAKGKGGSMHMYAKNFYGGNGIVGAQVPLGAGIALACKYNGKDEVCLTLYGDGAANQGQIFEAYNM
AALWKLPCIFICENNRYGMGTSVERAAASTDYYKRGDFIPGLRVDGMDILCVREATRFAAAYCRSGKGPI
LMELQTYRYHGHSMSDPGVSYRTREEIQEVRSKSDPIMLLKDRMVNSNLASVEELKEIDVEVRKEIEDAA
QFATADPEPPLEELGYHIYSSDPPFEVRGANQWIKFKSVS
SEQ ID NO. 40
Amino Acid
Pyruvate dehydrogenase E1 component subunit beta
Homo sapiens
MAAVSGLVRRPLREVSGLLKRRFHWTAPAALQVTVRDAINQGMDEELERDEKVFLLGEEVAQYDGAYKVS
RGLWKKYGDKRIIDTPISEMGFAGIAVGAAMAGLRPICEFMTFNFSMQAIDQVINSAAKTYYMSGGLQPV
PIVFRGPNGASAGVAAQHSQCFAAWYGHCPGLKVVSPWNSEDAKGLIKSAIRDNNPVVVLENELMYGVPF
EFPPEAQSKDFLIPIGKAKIERQGTHITVVSHSRPVGHCLEAAAVLSKEGVECEVINMRTIRPMDMETIE
ASVMKTNHLVTVEGGWPQFGVGAEICARIMEGPAFNFLDAPAVRVTGADVPMPYAKILEDNSIPQVKDII
FAIKKTLNI
SEQ ID NO. 41
Amino Acid
pyruvate dehydrogenase E1 component subunit alpha
Mus musculus
MRKMLAAVSRVLAGSAQKPASRVLVASRNFANDATFEIKKCDLHRLEEGPPVTTVLTREDGLKYYRMMQT
VRRMELKADQLYKQKIIRGFCHLCDGQEACCVGLEAGINPTDHLITAYRAHGFTFTRGLPVRAILAELTG
RRGGCAKGKGGSMHMYAKNFYGGNGIVGAQVPLGAGIALACKYNGKDEVCLTLYGDGAANQGQIFEAYNM
AALWKLPCIFICENNRYGMGTSVERAAASTDYYKRGDFIPGLRVDGMDILCVREATKFAAAYCRSGKGPI
LMELQTYRYHGHSMSDPGVSYRTREEIQEVRSKSDPIMLLKDRMVNSNLASVEELKEIDVEVRKEIEDAA
QFATADPEPPLEELGYHIYSSDPPFEVRGANQWIKFKSVS
SEQ ID NO. 42
Amino Acid
pyruvate dehydrogenase E1 component subunit beta
Mus musculus
MAVVAGLVRGPLRQASGLLKRRFHRSAPAAVQLTVREAINQGMDEELERDEKVFLLGEEVAQYDGAYKVS
RGLWKKYGDKRIIDTPISEMGFAGIAVGAAMAGLRPICEFMTFNFSMQAIDQVINSAAKTYYMSAGLQPV
PIVFRGPNGASAGVAAQHSQCFAAWYGHCPGLKVVSPWNSEDAKGLIKSAIRDNNPVVMLENELMYGVAF
ELPAEAQSKDFLIPIGKAKIERQGTHITVVAHSRPVGHCLEAAAVLSKEGIECEVINLRTIRPMDIEAIE
ASVMKTNHLVTVEGGWPQFGVGAEICARIMEGPAFNFLDAPAVRVTGADVPMPYAKVLEDNSVPQVKDII
FAVKKTLNI
SEQ ID NO. 43
Amino Acid
pyruvate dehydrogenase alpha subunit E1 alpha
Saccharomyces cerevisiae
MLAASFKRQPSQLVRGLGAVLRTPTRIGHVRTMATLKTTDKKAPEDIEGSDTVQIELPESSFESYMLEPP
DLSYETSKATLLQMYKDMVIIRRMEMACDALYKAKKIRGFCHLSVGQEAIAVGIENAITKLDSIITSYRC
HGFTFMRGASVKAVLAELMGRRAGVSYGKGGSMHLYAPGFYGGNGIVGAQVPLGAGLAFAHQYKNEDACS
FTLYGDGASNQGQVFESFNMAKLWNLPVVFCCENNKYGMGTAASRSSAMTEYFKRGQYIPGLKVNGMDIL
AVYQASKFAKDWCLSGKGPLVLEYETYRYGGHSMSDPGTTYRTRDEIQHMRSKNDPIAGLKMHLIDLGIA
TEAEVKAYDKSARKYVDEQVELADAAPPPEAKLSILFEDVYVKGTETPTLRGRIPEDTWDFKKQGFASRD
SEQ ID NO. 44
Amino Acid
pyruvate dehydrogenase beta subunit (E1 beta)
Saccharomyces cerevisiae
MFSRLPTSLARNVARRAPTSFVRPSAAAAALRFSSTKTMTVREALNSAMAEELDRDDDVFLIGEEVAQYN
GAYKVSKGLLDRFGERRVVDTPITEYGFTGLAVGAALKGLKPIVEFMSFNFSMQAIDHVVNSAAKTHYMS
GGTQKCQMVFRGPNGAAVGVGAQHSQDFSPWYGSIPGLKVLVPYSAEDARGLLKAAIRDPNPVVFLENEL
LYGESFEISEEALSPDFTLPYKAKIEREGTDISIVTYTRNVQFSLEAAEILQKKYGVSAEVINLRSIRPL
DTEAIIKTVKKTNHLITVESTFPSFGVGAEIVAQVMESEAFDYLDAPIQRVTGADVPTPYAKELEDFAFP
DTPTIVKAVKEVLSIE

1. A method of the wax biosynthesis comprising the step of transforming an algal cell with one or more polynucleotide sequences operably linked to a promoter that expresses a heterologous fatty acyl-CoA reductase (FAR), and a heterologous wax synthase (WS) wherein said FAR and WS peptides operate to biosynthesize wax esters.

2. The method of claim 1 wherein said step of transforming comprises the step of transforming a Chlamydomonas reinhardtii cell.

3-4. (canceled)

5. The method of claim 1 wherein said heterologous fatty acyl-CoA reductase (FAR) is selected from the group consisting of:
a heterologous fatty acyl-CoA reductase (FAR) according to amino acid sequence SEQ ID NO. 1; and
a heterologous fatty acyl-CoA reductase (FAR) according to amino acid sequence SEQ ID NO. 5.

6. (canceled)

7. The method of claim 5 wherein said heterologous wax synthase (WS) is selected from the group consisting of:
a heterologous wax synthase (WS) according to amino acid sequence SEQ ID NO. 2; and
a heterologous wax synthase (WS) according to amino acid sequence SEQ ID NO. 6;

8. (canceled)

9. The method of claim 7 and further comprising the step of producing an acyl species having an identity of C20:1/C22:0.

10. The method of claim 7 wherein said biosynthesized wax ester comprises a C42:1 wax ester.

11. The method of claim 10 and further comprising the step of culturing the transformed algal cell and feeding said algal culture a quantity of 1-dodecanol.

12. The method of claim 11 and further comprising the step of biosynthesizing a C34:2 wax ester after feeding said algal culture a quantity of 1-dodecanol.

13. The method of claim 11 and further comprising the step of producing hydroxylated triacylglycerol species (ETAG, OHTAG) in said algal culture after feeding said algal culture a quantity of 1-dodecanol.

14. The method of claim 1 wherein said heterologous wax synthase (WS) that biosynthesizes wax esters from said acyl alcohol comprises a heterologous acyl-CoA:diacylglycerol acyltransferase that biosynthesizes wax esters from said acyl alcohol selected from the group consisting of: SEQ ID NO. 9, SEQ ID NO. 11, and 13.

15. (canceled)

16. The method of claim 1 and further comprising the step of downregulating the expression of diacylglycerol acyl transferase (DGAT2) in said transformed algal cell.

17. (canceled)

18. The method of claim 1 and further comprising the step of downregulating the expression of very long chain fatty acid (VLCFA) elongases in said transformed algal cell.

19. (canceled)

20. The method of claim 1 and further comprising the step of downregulating the expression of fatty acid elongase (FAE).

21. (canceled)

22. The method of claim 1 and further comprising the step of increasing expression of pyruvate dehydrogenase in said transformed algal cell to increase production of acetyl-CoA.

23. The method of claim 22 wherein said step of increasing expression of pyruvate dehydrogenase in said transformed algal cell to increase production of acetyl-CoA comprises the step of transforming said algal cell to express a heterologous pyruvate dehydrogenase complex selected from the group of amino acid sequences SEQ ID NOs. 38-43.

24. (canceled)

25. The method of claim 1 and further comprising the step of transforming said algal cell to express a heterologous cyanobacterial fructose-1,6-/sedoheptulose-1,7-bisphosphatase (fII).

26. The method of claim 1 wherein said step of transforming said algal cell to express a heterologous cyanobacterial fructose-1,6-/sedoheptulose-1,7-bisphosphatase (fII) comprises the step of transforming said algal cell to express a heterologous cyanobacterial fructose-1,6-/sedoheptulose-1,7-bisphosphatase (fII) according to amino acid sequence SEQ ID NO. 24.

27. The method of claim 1 and further comprising the step of culturing the transformed algal cell under low nitrogen conditions.

28. (canceled)

29. A method of novel wax biosynthesis in algae comprising the steps of:
transforming an algal cell with one or more polynucleotide sequences operably linked to a promoter that expresses:
a heterologous fatty acyl-CoA reductase from Simmondsia chinensis according to amino acid sequence SEQ ID NO. 1, that reduces long-chain-fatty-acyl-CoA to acyl alcohol;
a heterologous wax synthase from Simmondsia chinensis according to amino acid sequence SEQ ID NO. 2, that biosynthesizes wax esters from said acyl alcohol;
culturing said algal cell; and
harvesting the biosynthesized wax esters from the algal cell culture.

30-48. (canceled)

49. A method of novel wax biosynthesis in algae comprising the steps of:
transforming an algal cell with one or more polynucleotide sequences operably linked to a promoter that expresses:
a heterologous fatty acyl-CoA reductase from Euglena gracilis according to amino acid sequence SEQ ID NO. 5, that reduces long-chain-fatty-acyl-CoA to acyl alcohol; and
a heterologous wax synthase from Euglena gracilis according to amino acid sequence SEQ ID NO. 6, that biosynthesizes wax esters from said acyl alcohol;
culturing said algal cell; and
harvesting the biosynthesized wax esters from the algal cell culture.

50-83. (canceled)
